(19) United States
(12) Patent Application Publication
(10) Pub. No.: US 2026/0364022 A1
(43) Pub. Date: Nov. 19, 2026

(54) LIPID NANOPARTICLE FORMULATIONS FOR MRNA DELIVERY TO LANGERHANS CELLS

(71) Applicant: CUTANOS GMBH, Vienna (AT)

(72) Inventors: Robert Carsten Willi WAWRZINEK, Vienna (AT); Christoph Johannes Heinrich RADEMACHER, Vienna (AT); Federica QUATTRONE, Vienna (AT); Klara KLEIN-EBERL, Vienna (AT); Litty JOHNSON, Vienna (AT); Nowras RAHHAL, Vienna (AT); Mareike RENTZSCH, Berlin (DE)

(73) Assignee: CUTANOS GMBH, Vienna (AT)

(21) Appl. No.: 19/120,287

(22) PCT Filed: Oct. 13, 2023
(86) PCT No.: PCT/EP2023/078568

§ 371 Date: Apr. 11, 2025

(30) Foreign Application Priority Data
Oct. 14, 2022 (EP) 22201740.2

(51) Int. Cl. A61K 9/51; A61K 9/00; A61K 47/34; A61K 48/00

(52) U.S. Cl. CPC A61K 9/5123; A61K 9/0019; A61K 9/5192; A61K 47/34; A61K 48/0025; A61K 48/0075; A61K 48/0091

(57) ABSTRACT

The present invention relates to pharmaceutical compositions comprising at least one lipid nanoparticle (LNP) specific for targeting Langerhans cells (LC) wherein the LNP encapsulates at least one mRNA, is capable of specifically binding to the receptor Langerin and facilitates Langerin-mediated uptake and intracellular delivery of said mRNA molecule and its translation into at least one protein or peptide. Further envisaged is the pharmaceutical composition for use in the treatment of cancer, of an autoimmune disease, of a bacterial infection, of a viral infection, of a fungal infection or of a graft-vs. host disease, of a local or systemic inflammation, of an allergy, or for hyposensitization.

### FIELD OF THE INVENTION

The present invention relates to pharmaceutical compositions comprising at least one lipid nanoparticle (LNP) specific for targeting Langerhans cells (LC) wherein the LNP encapsulates at least one mRNA, is capable of specifically binding to the receptor Langerin and facilitates Langerin-mediated uptake and intracellular delivery of said mRNA molecule and its translation into at least one protein or peptide. Further envisaged is a pharmaceutical composition for use in the treatment of cancer, of an autoimmune disease, of a bacterial infection, of a viral infection, of a fungal infection or of a graft-vs. host disease, of a local or systemic inflammation, of an allergy, or for hyposensitization.

### BACKGROUND OF THE INVENTION

The approval of recent mRNA-based vaccines against SARS-CoV2 marks a hallmark in development of a new generation of therapeutic and prophylactic approaches, employing protein antigen-encoding nucleic acids. The use of mRNA in such a way requires the delivery to muscle and dendritic cells (DCs). Due to its instability and immunogenicity, the mRNA must be encapsulated into a protected layer. Herein, numerous lipid-based formulations, known as lipid nanoparticles (LNPs) have been developed over the last years. These formulations aim to meet several requirements, such as ensuring mRNA stability, uptake into cells and promotion of mRNA translation into protein.

Despite the success of the approved mRNA-based vaccines, inherent shortcomings of contemporary LNP formulations are the focus of current development efforts in academia and industry alike: since an immune response against an antigen is mainly elicited by special antigen-presenting immune cells, namely dendritic cells (DCs) and macrophages, the deposition of mRNA-loaded LNPs in muscle tissue is rather inefficient. This is due to the low immune cell density in this tissue, reducing the probability of antigen uptake by DCs, after the protein has been produced by, for example, muscle cells. This low probability of antigen loading into DCs requires the administration of a certain mRNA dose in order to elicit the desired immune response. However, since a substantial amount of mRNA is not involved in this process, but still causes inflammation, this dose is limited, before immune reactions to either the RNA and/or the lipid composition or its reaction products reach an unacceptable limit.

In contrast to muscle tissue, the skin, as the protective interfaces between the outside world and the body is equipped with a number of potent mechanisms to tackle environmental antigens, including chemicals, bacteria, and pathogens. To fulfil this function, different layers of the skin contain a higher density of different subsets of DCs than muscles. These skin DCs play a critical role in guarding the host against invading pathogens, while promoting tolerance to innocuous or self-antigens. Therefore, the skin is a promising target in immune modulating strategies.

In addition, the frequent T cell-DC contacts during T cell scanning of DCs in lymphoid organs, i.e., in the absence of cognate antigen, induce a basal activation level in T cells required for rapid responsiveness to subsequent encounters with foreign antigen during inflammation. Pathogen invasion together with proinflammatory signals typically drive a full functional maturation of skin DCs. Beyond the homeostatic differentiation program, the cells also upregulate the expression of costimulatory molecules and, in particular, proinflammatory cytokines. Together, these promote clonal expansion of naïve antigen-specific T cells and instruct the T cells to acquire appropriate effector functions specifically tailored to eliminate the invading pathogen (sensitizing function). Thus, immature DCs in the periphery have a sentinel function and are capable of antigen capture, antigen processing and subsequent peptide-presentation via MHC complexes. They also have a migratory function and provide for the antigen transport to the lymph nodes. Mediated by high levels of surface MHC I and II/peptide complexes, DC-T cell interaction leads to Th1/Th2/Th17/Treg instruction, or T cell deletion and anergy, or cytotoxic T-lymphocyte (CTL, T-killer cell) activation. According to current reports (Doebel et al., 2017, Trends Immunol, 38, 11, 817-828) the groups of DCs in the skin can be subdivided into several DC subsets. The DCs can either be epidermal Langerhans cells (LCs) and dermal DCs. The LCs express the pattern recognition receptor Langerin (CD207), which is known to be involved in the Ca²⁺-dependent recognition of both pathogen- and self-associated glycans including heparin-like oligosaccharides (Munoz-Gracia et al., J. Am. Chem. Soc. 2015, 137, 12, 4100-10.). Since Langerin displays an expression profile highly restricted to skin-residing Langerhans cells, it represents an attractive target for cell-specific vaccine approaches or the development of innovative therapies against autoimmune disorders and allergies.

Previous approaches targeting skin cells with LNPs, have shown that mRNA expression can be observed in a plethora of cells, of which a mere fraction are Langerhans cells (Blakney et al., 2021, J Control Release, 330:1250-1261; Blakney et al., 2019, ACS Nano, 13, 5, 5920-5930).

However, it is difficult to specifically target LNPs to Langerhans cells since the carbohydrate binding sites of Langerin is highly solvent exposed and hydrophilic. Consequently, interactions with mono- and oligosaccharides are typically characterized by low affinities in the millimolar range. Furthermore, the recognition process is highly promiscuous as other C-type lectins are expressed on other DCs and bind several mono- or oligosaccharides and vice versa. In this context, Aretz et al., 2014 noted that the structure-based in silico analysis of 21 X-ray structures corroborated the classification of C-type lectins as undruggable and challenging targets for traditional approaches in drug discovery (Aretz et al., 2014, Front Immunol, 5, Article 323). However, in 2019, a specific, small molecule-targeting ligand was described, which advantageously, when presented on the surface of liposomes enabled selective uptake by Langerhans cells (Wamhoff et al., 2019, ACS Central Sci., 5, 808.; Bellmann et al., 2020 J. Invest. Dermatol., 141, 84.; Schulze et al., Biochemistry 2019, 58, 2576.). Yet there is still no pharmaceutical composition which successfully delivers nucleic acids into Langerhans cells and induces the production of polypeptides.

There is hence a clear need for an efficient formulation which effectively targets Langerhans cells, introduces nucleic acids into these cells and promotes translation.

### OBJECTS AND SUMMARY OF THE INVENTION

The present invention addresses these needs and provides a pharmaceutical composition comprising at least one lipid nanoparticle (LNP) specific for targeting Langerhans cells (LC) wherein the LNP encapsulates at least one mRNA, is capable of specifically binding to the receptor Langerin and facilitates Langerin-mediated uptake and intracellular delivery of said mRNA and its translation into at least one protein or peptide.

The inventors surprisingly found that the LNP according to the present invention, e.g. an LNP comprising one or more targeting lipids, one or more ionizable lipids, one or more helper lipids, and one or more structural lipids, optionally also one or more stealth lipids, is capable of (i) enabling Langerhans cell-specific mRNA delivery, (ii) facilitating efficient mRNA translation into antigen within primary Langerhans cells and, as a result, (iii) allowing for the presentation of antigen peptides to T cells by Langerhans cells. In particular, the pharmaceutical composition according to the present invention comprises LNPs which contain a targeting lipid that allows for specific uptake by primary Langerhans cells and lead to efficient mRNA translation. In particular, the inventors identified a specific molar ratio range for the targeting lipid within the LNP lipid composition, which significantly enhances these steps, as shown in FIG. 8. The present invention thus advantageously solves the critical problem of inducing antigen production in immune-competent Langerhans cells. Without wishing to be bound by theory, it is assumed that the Langerin receptor-mediated uptake of LNPs containing a targeting lipid (targeted LNPs) according to the present invention increases the accumulation of mRNA in endosomal compartments, which is assumed to lead to its enhanced endosomal escape into the cytosol. This mechanism is hypothesized to be similar to the asialoglycoprotein receptor (ASGPR, or Ashwell-Morell receptor)-mediated uptake for which efficient mRNA delivery to a different target tissue such as liver tissue has been described. Subsequent to the mRNA delivery an efficient mRNA-to-protein translation can ensue.

As has been found by the present inventors, conventional LNPs, which do not bear a suitable targeting lipid, i.e. non-targeted LNPs, and which rely on alternative uptake mechanisms, do not undergo a Langerin-mediated uptake in Langerhans cells and do not show an associated efficient endosomal escape mechanism, whereas the LNPs according to the present invention successfully rely on a Langerin-mediated uptake, show an associated efficient endosomal escape mechanism and thereby allow for cell-specific mRNA delivery and translation in Langerhans cells, in particular in skin-residing LCs.

A further advantage of the present invention is that due to low off-target uptake by other cells than LCs and due to the additional, efficient endosomal escape, high translation rates become feasible. This can in consequence be used for a significant dose reduction for the formulations of this invention in comparison to non-targeted LNPs.

In consequence, the pharmaceutical compositions according to the present invention can be used to very effectively initiate an immune response in vivo to the antigen encoded by the delivered mRNA, e.g. in the form of therapeutic or prophylactic vaccines, or to induce peripheral or central tolerance.

In a preferred embodiment said LNP comprises a targeting lipid, which is capable of specifically binding to the receptor Langerin, of the general formula (I)

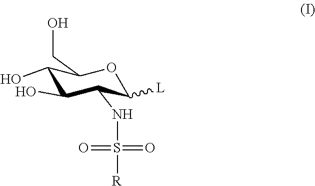

wherein L is a lipid, a modified lipid, such as a phospholipid, 1,2-distearoyl-sn-glycero-3-phosphoethanolamine (DSPE), oxyglutaryl aminopropyl polyethyleneglycol-carbamyl distearoylphosphatidyl-ethanolamine (DSPE-PEG), a membrane lipid, or a modified phosphatidylcholine; and
wherein R is a phenyl, a mono-, di- or trisubstituted phenyl,
wherein substituents of the phenyl are independently selected from the group consisting of
—NH₂, —OH, —OCH₃, —C(O)CH₃, C(O)NH₂, —C(O)NHCH₃, —CH₂OH —NHC(O)CH₃, —F, —Cl, —Br, —NO₂, —CN, C₁-C₄ alkyl, naphtyl and phenyl.

In a further preferred embodiment, the LNP additionally comprises at least one stealth lipid. It is particularly preferred that the stealth lipid is DSPE-PEG (N-(Methylpolyoxyethylene oxycarbonyl)-1,2-distearoyl-sn-glycero-3-phosphoethanolamine), DMG-PEG (1,2-dimyristoyl-rac-glycero-3-methoxypolyethylene glycol) or DSG-PEG (1,2-Distearoyl-rac-glycero-3-methylpolyoxyethylene), or, even more preferably, DSPE-PEG2000 (N-[Carbonyl(methoxypolyethylene glycol)]-1,2-distearoyl-sn-glycero-3-phosphoethanolamine).

In yet another preferred embodiment the LNP additionally comprises at least one helper lipid. It is particularly preferred that the helper lipid is DSPC (1,2-Distearoyl-sn-glycero-3-phosphocholine), DSPE (1,2-Distearoyl-sn-glycero-3-phosphoethanolamine) or DOPE (1,2-Dioleoyl-sn-glycero-3-phosphoethanolamine).

In a further preferred embodiment, the LNP additionally comprises at least one structural lipid. It is particularly preferred that the structural lipid is cholesterol or a cholesterol analog or a combination thereof.

In another preferred embodiment the LNP according to the present invention additionally comprises at least one ionizable lipid, preferably DLin-MC3-DMA ((6Z,9Z,28Z,31Z)-Heptatriaconta-6,9,28,31-tetraen-19-yl 4-(dimethylamino) butanoate), SM-102 (8-[(2-hydroxyethyl)[6-oxo-6-(undecyloxy)hexyl]amino]-octanoic acid, 1-octylnonyl ester), ALC-0315 (2-hexyl-decanoic acid, 1,1′-[[(4-hydroxybutyl) imino]di-6,1-hexanediyl]ester), or DODMA (1,2-Dioleyloxy-3-dimethylamino-propane).

In another preferred embodiment of the pharmaceutical composition according to the present invention the mRNA is unmodified or modified. The modified mRNA is designed to optimize stability, translation efficiency or immunogenicity.

In a preferred embodiment wherein the targeting lipid is of one of the following formulae:

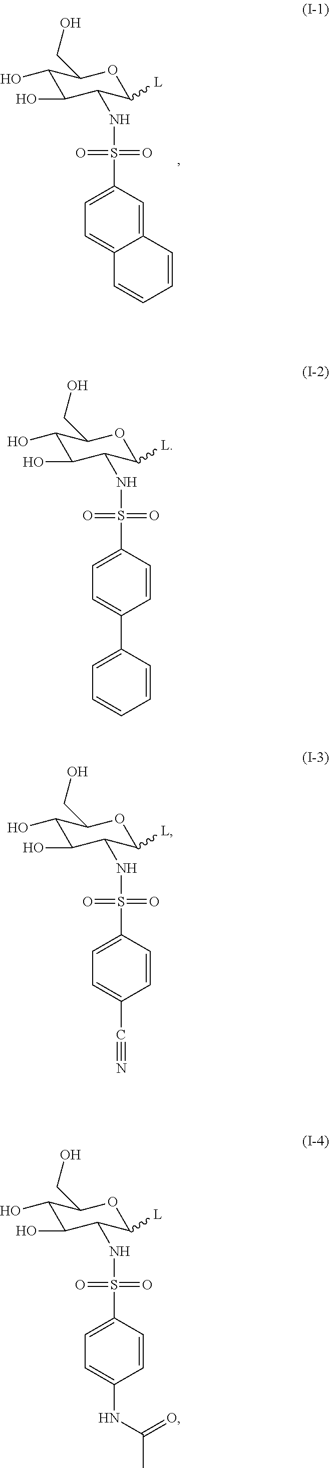

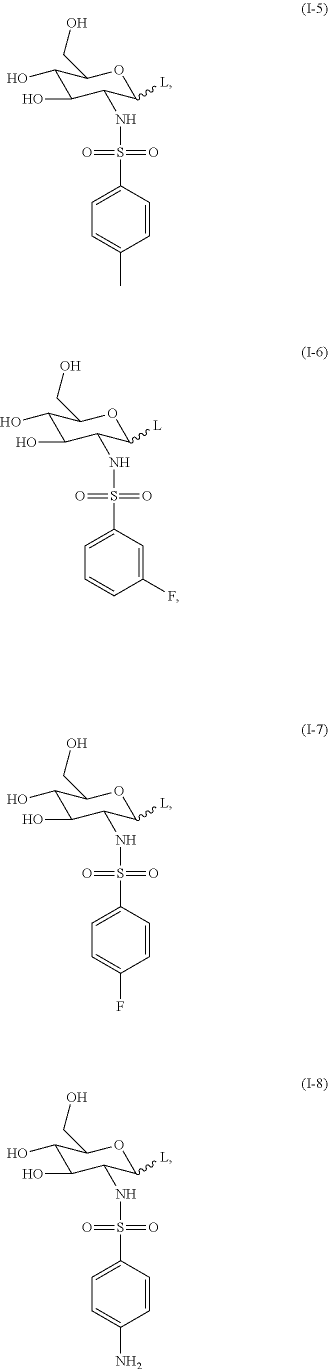

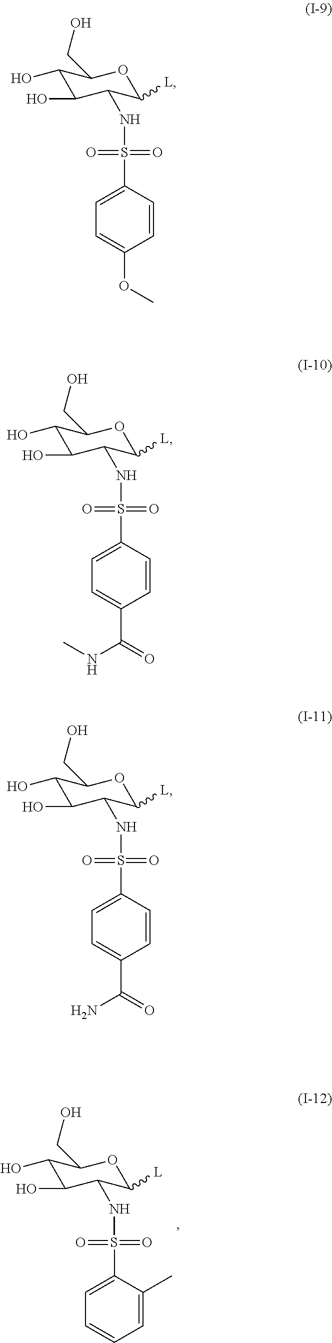

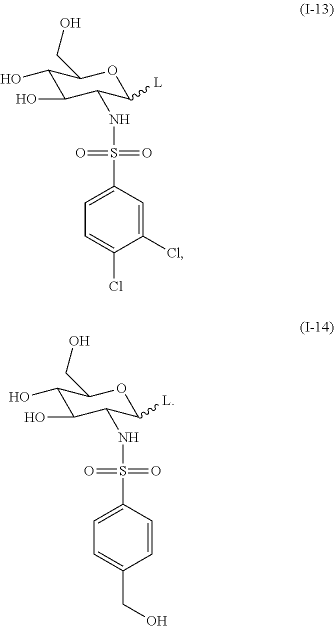

In a further preferred embodiment L is of the following formula (II):

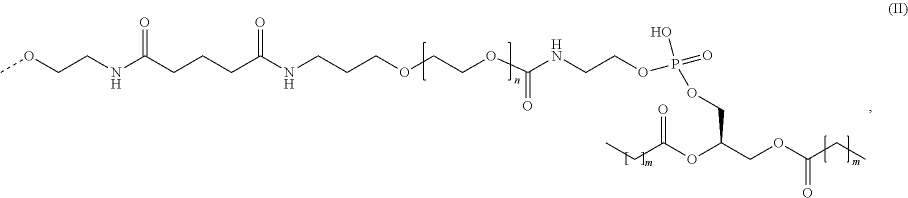

wherein n is an integer from 0 to 150 and m is an integer from 1 to 30.

In a particularly preferred embodiment of the pharmaceutical composition of the present invention the targeting lipid is of the following formula (III)

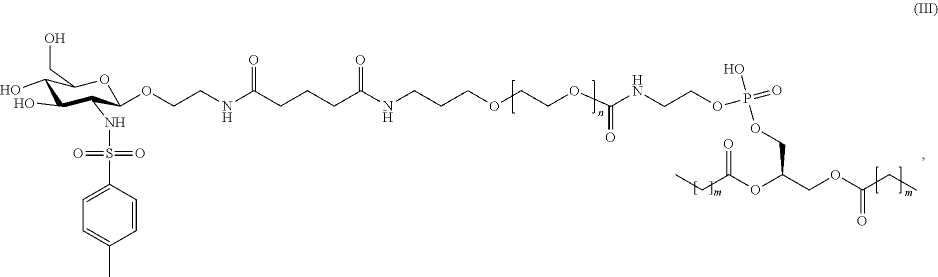

wherein n is an integer from 0 to 150 and m is an integer from 1 to 30.

In yet another preferred embodiment of the invention the LNP has a size of about 30 to 250 nm.

In a further preferred embodiment, the pharmaceutical composition comprises a solvent or a combination of a solvent with a further compound, or is provided as a dry composition, preferably as powder composition.

It is particularly preferred that said solvent is H₂O, an aqueous sucrose solution, a phosphate buffered saline, an aqueous sodium chloride solution, a tricine buffer, or HEPES buffer.

It is further particularly preferred that said further compound is DMSO, propylene glycol, or oleic acid, more preferably DMSO in a concentration of up to 10%.

According to another specific embodiment of the present invention the amount of the targeting lipids in the LNP is about 0.01 to 5 mol %. It is particularly preferred that the amount of the targeting lipids in the pharmaceutical composition is about 0.1 to 3 mol %, more preferably about 0.25 to 2 mol %.

In yet another preferred embodiment the amount of the stealth lipids in the LNP is about 0 to 5 mol %. In a particularly preferred embodiment, the amount of the stealth lipids in the pharmaceutical composition is about 0.1 to 2 mol %.

In another preferred embodiment the amount of the helper lipids in the LNP is about 5 to 15 mol %. In a particularly preferred embodiment, the amount of the helper lipids is about 10 mol %.

In yet another preferred embodiment of the present invention the amount of the structural lipids in the LNP is about 20 to 60 mol %. In a particularly preferred embodiment, the amount of the structural lipids in the LNP is 35 to 40 mol %.

In a further preferred embodiment of the present invention the amount of the ionizable lipids or of the combination of ionizable lipids in the LNP is about 10 to 70 mol %, more preferably about 15 to 60 mol %, even more preferably about 20 to 55 mol %.

In another embodiment of the present invention said mRNA encodes for a pharmaceutically or immunologically active compound.

It is preferred that the mRNA encodes for any one of: (i) a cancer antigen or epitope; (ii) an autoimmune disease antigen or epitope; (iii) a bacterial antigen or epitope; (iv) a viral antigen or epitope; (v) a parasitic antigen or epitope; or (vi) an allergen, or an epitope of an allergen.

In yet another embodiment said pharmaceutical composition is suitable for topical, intradermal, transdermal, transfollicular, subcutaneous, intramuscular, intravenous, oral, sublingual, buccal, ophthalmic, otic, nasal, vaginal, or rectal administration or via inhalation.

In a further embodiment said pharmaceutical composition is provided as a patch, nanopatch, cream, ointment, paste, gel, powder, lotion, tape, film, tablet, spray, suppository, or in the form of a solution for injection.

In a further embodiment the pharmaceutical composition is to be administered without or with a medical device, such as a needle, a microneedle device, a vaccination gun, a plaster, or an inhaler.

In a preferred embodiment the pharmaceutical composition according to the present invention is for use in the treatment or prevention of cancer, of an autoimmune disease, of a bacterial infection, of a viral infection, of a fungal infection or of a graft-vs. host disease, of a local or systemic inflammation, of an allergy, or for hyposensitization.

In a further aspect the present invention relates to a method of treatment or prevention of cancer, of an autoimmune disease, of a bacterial infection, of a viral infection, of a fungal infection, of a graft-vs. host disease, of a local or systemic inflammation, of an allergy, or for hyposensitization comprising administering to a subject a therapeutically effective amount of the pharmaceutical according to the present invention.

In a preferred embodiment of the method of treatment said administration is a topical, intradermal, transdermal, transfollicular, subcutaneous, intramuscular, intravenous, oral, sublingual, buccal, ophthalmic, otic, nasal, vaginal, or rectal administration or via inhalation.

### BRIEF DESCRIPTION OF THE DRAWINGS

FIG. 1—t-LNP (1 to 4) ameliorate LNP uptake and mRNA translation in primary LCs. Targeted LNPs (t-LNP 1, t-LNP 2, t-LNP 3, t-LNP 4) were compared to the equivalent variants not bearing a targeting ligand (non-targeted LNPs: nt-LNP 1, nt-LNP 2, nt-LNP 3, nt-LNP 4). The preparation of the LNPs in this study as well as the experimental protocol to conduct the experiment are described in the Examples 1, 2, 3, and 4. The compositions and physical characterisation of the LNPs used in FIG. 1 are provided in Table 1 depicted in FIG. 9. Murine epidermal cell (EC) suspensions were stimulated with GFP-mRNA laden t-LNP, nt-LNP (0.25 μg/mL mRNA concentration) or were left untreated for 16 h. FIG. 1A-1F: The LNP uptake (x-axis, PEG-AF647 signal) and mRNA translation (y-axis, GFP signal) by LCs (identified as MHC-II⁺, viable single cells) were analysed by flow cytometry. Targeted LNPs (i.e. FIG. 1A,1B,1C: t-LNP 1, t-LNP 2; FIG. 1D.1E.1F: t-LNP 3, t-LNP 4) show superior delivery to LCs and result in higher mRNA translation compared to their non-targeted counterparts (i.e. FIG. 1A,1B,1C: nt-LNP 1, nt-LNP 2; FIG. 1D, 1E, 1F: nt-LNP 3, nt-LNP 4). FIG. 1A: Bar graph shows the percentages of mRNA translating LCs after stimulation with t-LNP 1, nt-LNP 1, t-LNP 2 and nt-LNP 2. FIG. 1B: FACS plots depicting mRNA translation (y-axis, GFP signal) and LNP uptake (x-axis, PEG-AF647 signal) after stimulation with t-LNP 1 and nt-LNP 1. FIG. 1C: FACS plots depicting mRNA translation (y-axis, GFP signal) and LNP uptake (x-axis, PEG-AF647 signal) after stimulation with t-LNP 2 and nt-LNP 2. FIG. 1D: Bar graph shows the percentages of mRNA translating LCs after stimulation with t-LNP3, nt-LNP3, t-LNP4 and nt-LNP4. FIG. 1E: FACS plots depicting mRNA translation (y-axis) and LNP uptake (x-axis) after stimulation with t-LNP 3 and nt-LNP 3. FIG. 1F: FACS plots depicting mRNA translation (y-axis) and LNP uptake (x-axis) after stimulation with t-LNP 4 and nt-LNP 4.

FIG. 2—t-LNP 3 successfully delivers OVA-encoding mRNA to primary LCs and enhances antigen presentation to OT-I T cells in vitro. Co-cultures of EC suspension, prepared from huLang mice (as described in Example 3), and CFSE labelled naïve CD8+OT-I T cells, isolated from the spleen and lymph nodes of transgenic OT-I mice (as described in Example 5), were setup at a ratio of 20:1 (EC:OT-I T cells) and stimulated with t-LNP 3 or nt-LNP 3 formulations containing mRNA encoding for the model antigen ovalbumin (OVA) at an mRNA concentration of 0.1, 0.25 or 1 μg/ml. The molar compositions and physical characterisation of the LNPs used in FIG. 2 are provided in Table 2 (depicted in FIG. 10). Untreated co-cultures served as negative controls, while stimulation with SIINFEKL peptide (10 μg/ml) was used as a positive control. After 72 h, flow cytometric analysis (as described in Example 7) of OT-I T cell proliferation was performed by detection of dilution of the CFSE proliferation dye. FIG. 2A: Bar graph shows the percentages of proliferating cells (CFSE^{low}) among OT-I T cells (gated as viable CD45.1+ TCR Vβ5.1, 5.2+CD8+ cells). FIG. 2B: FACS plots depict the CFSE dilution (x-axis) in CD8+ (y-axis) OT-I T cells in the different co-culture conditions.

FIG. 3—t-LNP 3 successfully delivers OVA-encoding mRNA to primary LCs and enhances antigen presentation to OT-II T cells In vitro. Co-cultures of EC suspension, prepared from hulang mice (as described in Example 3), and CFSE labelled naïve CD4+OT-II T cells, isolated from the spleen and lymph nodes of transgenic OT-II mice (as described in Example 5), were setup at a ratio of 20:1 (EC:OT-II T cells) and stimulated with t-LNP 3 or nt-LNP 3 formulations containing mRNA encoding for OVA at an mRNA concentration of 0.1, 0.25 or 1 μg/ml. The molar compositions and physical characterisation of the LNPs used in FIG. 3 are provided in Table 2 (depicted in FIG. 10). Untreated co-cultures served as negative controls, while stimulation with OVA₃₂₃₋₃₃₉ peptide (10 μg/ml) was used as a positive control. After 72 h, flow cytometric analysis (as described in Example 7) of OT-II T cell proliferation was performed by detection of dilution of the CFSE proliferation dye. FIG. 3A: Bar graph shows the percentages of proliferating cells (CFSE^{low}) among OT-II T cells (gated as viable CD45.1+ CD3+CD4+ cells). FIG. 3B: FACS plots depict the CFSE dilution (x-axis) in CD4+(y-axis) OT-II T cells in the different co-culture conditions.

FIG. 4—t-LNP 1 successfully delivers OVA-encoding mRNA to primary LCs and enhances antigen presentation to OT-I T cells In vitro. Co-cultures of EC suspension, prepared from huLang mice (as described in Example 3), and CFSE labelled naïve CD8+OT-I T cells, isolated from the spleen and lymph nodes of transgenic OT-I mice (as described in Example 5), were setup at a ratio of 20:1 (EC:OT-I T cells) and stimulated with t-LNP 1 or nt-LNP 1 formulations containing mRNA encoding for OVA at an mRNA concentration of 1 μg/ml. The molar compositions and physical characterisation of the LNPs used in FIG. 4 are provided in Table 2 (depicted in FIG. 10). Untreated co-cultures served as negative controls, while stimulation with SIINFEKL peptide (100 μg/ml) was used as a positive control. After 72 h, flow cytometric analysis (as described in Example 7) of OT-I T cell proliferation was performed by detection of dilution of the CFSE proliferation dye. FIG. 4A: Bar graph shows the percentages of proliferating cells (CFSE^{low}) among OT-I T cells (gated as viable CD90.2+CD8+ cells). FIG. 4B: FACS plots depict the CFSE dilution (x-axis) in CD8+(y-axis) OT-I T cells under the different co-culture conditions.

FIG. 5—t-LNP 1 successfully delivers OVA-encoding mRNA to primary LCs and enhances antigen presentation to OT-II T cells In vitro. Co-cultures of EC suspension, prepared from huLang mice (as described in Example 3), and CFSE labelled naïve CD4+OT-II T cells, isolated from the spleen and lymph nodes of transgenic OT-II mice (as described in Example 5), were setup at a ratio of 20:1 (EC:OT-II T cells) and stimulated with t-LNP 1 or nt-LNP 1 formulations containing mRNA encoding for OVA at an mRNA concentration of 1 μg/ml. The molar compositions and physical characterisation of the LNPs used in FIG. 5 are provided in Table 2 (depicted in FIG. 10). Untreated co-cultures served as negative controls, while stimulation with OVA₃₂₃₋₃₃₉ peptide (100 μg/ml) was used as a positive control. After 72 h, flow cytometric analysis (as described in Example 7) of OT-II T cell proliferation was performed by detection of dilution of the CFSE proliferation dye. FIG. 5A: Bar graph shows the percentages of proliferating cells (CFSE^{low}) among OT-II T cells (gated as viable CD90.2+CD4+ cells). FIG. 5B: FACS plots depict the CFSE dilution (x-axis) in CD4+(y-axis) OT-II T cells under the different co-culture conditions.

FIG. 6—Intradermally Injected t-LNP 3 successfully delivers OVA-encoding mRNA to primary LCs In vivo and elicits a superior antigen-specific T cell response in vitro. HuLang mice were intradermally (i.d.) injected into the ears (as described in Example 6) with 1 μg of OVA encoding mRNA encapsulated in t-LNP 3 or nt-LNP 3 formulations. The molar compositions and physical characterisation of the LNPs used in FIG. 6 are provided in Table 2 (depicted in FIG. 10). Three hours after the injection, EC suspensions were prepared from the mice and co-cultured with CFSE labelled naïve CD8+ OT-I T cells, isolated from the spleen and lymph nodes of transgenic OT-I mice (as described in Example 5), at a ratio of 20:1 (EC:OT-I T cells). EC suspension from untreated back skin was co-cultured with OT-I T cells as negative controls. After 72 h, flow cytometric analysis (as described in Example 7) of OT-I T cell activation (CD25, CD44 and CD69 expression) and proliferation (CFSE dilution) was performed. FIG. 6A: Bar graph shows the percentages of proliferating cells (CFSE^{low}) among OT-I T cells (gated as viable CD45.1+ TCR Vβ5.1, 5.2+CD8+ cells). FIG. 6B: Bar graphs show the percentages of activated cells (CD25+(left panel), CD44+(middle panel) or CD69+(right panel)) among OT-I T cells (gated as viable CD45.1+ TCR Vβ5.1, 5.2+CD8+ cells). Statistical analysis was performed by one-way ANOVA, levels of significance are defined as follows: *p<0.05 and **p<0.01.

FIG. 7—Chemical structure of Langerin-specific targeting ligand (free amine form). The chemical structure of the targeting ligand prior to coupling the ligand to a lipid is shown (as described in Example 1).

FIG. 8—mRNA translation in primary LCs decreases with an increasing molar ratio of targeting lipid. Targeted LNPs (t-LNP 3) were formulated with different molar ratios of targeting lipid (1.5%, 2.5%, 3.5%, 4.5%, 5.5%) and compared to each other for their mRNA translation efficiency in primary LCs. The formulation procedure of the LNPs in this study as well as the experimental protocol to conduct the experiment are described in the Examples 1, 2, 3 and 4. The molar compositions and physical characterisation of the LNPs used in FIG. 8 are provided in Table 3 (depicted in FIG. 11). Murine EC suspensions were stimulated with GFP-mRNA encapsulated t-LNPs (0.25 μg/mL mRNA concentration) or were left untreated. At 16 h post stimulation, the LNP uptake (x-axis, PEG-AF647 signal) and mRNA translation (y-axis, GFP signal) by LCs (identified as MHC-II⁺, viable single cells) were analysed by flow cytometry. mRNA translation decreases with increasing targeting lipid molar ratio and t-LNPs formulated with the lowest targeting lipid molar ratio (i.e. t-LNP 3-1.5%) showed the highest mRNA translation compared to the other ratios. FIG. 8A: Bar graph shows the percentages of GFP mRNA translating LCs. FIG. 8B: FACS plots depict LNP uptake (x-axis) and mRNA translation (y-axis) in LCs after stimulation with t-LNP 3 with an increasing molar ratio of the targeting lipid.

FIG. 9 shows Table 1, which provides details of the composition and physical characteristics of lipid nanoparticles containing GFP mRNA used in the studies depicted in FIG. 1.

FIG. 10 shows Table 2, which provides details of the composition and physical characteristics of lipid nanoparticles containing OVA mRNA used in the studies depicted in FIGS. 2-6.

FIG. 11 shows Table 3, which provides details of the composition and physical characteristics of lipid nanoparticles containing GFP mRNA used in the studies depicted in FIG. 8.

### DETAILED DESCRIPTION OF THE EMBODIMENTS

Although the present invention will be described with respect to particular embodiments, this description is not to be construed in a limiting sense. In the following definitions important for understanding the present invention are given.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention, the term “consisting of” or “essentially consisting of” is considered to be a preferred embodiment of the term “comprising of”. If hereinafter a group is defined to comprise at least a certain number of embodiments, this is meant to also encompass a group, which preferably consists of these embodiments only.

Furthermore, the terms “(i)”, “(ii)”, “(iii)” or “(a)”, “(b)”, “(c)”, “(d)”, or “first”, “second”, “third” etc. and the like in the description or in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms relate to steps of a method or use there is no time or time interval coherence between the steps, i.e. the steps may be carried out simultaneously or there may be time intervals of seconds, minutes, hours, days, weeks etc. between such steps, unless otherwise indicated.

It is to be understood that this invention is not limited to the particular methodology, protocols, reagents etc. described herein as these may vary. It is also to be understood that the terminology used herein is for the purpose of describing particular embodiments only and is not intended to limit the scope of the present invention that will be limited only by the appended claims.

Unless defined otherwise, all technical and scientific terms used herein have the same meanings as commonly understood by one of ordinary skill in the art.

Independent of the grammatical term usage, individuals with male, female or other gender identities are included within the term.

As has been set out above, the present invention concerns in one aspect a pharmaceutical composition comprising at least one lipid nanoparticle (LNP) specific for targeting Langerhans cells (LC), wherein the LNP encapsulates at least one mRNA, is capable of specifically binding to the receptor Langerin and facilitates Langerin-mediated uptake and intracellular delivery of said mRNA and its translation into at least one protein or peptide.

The term “lipid nanoparticle” or, abbreviated, “LNP” refers to a nanoparticle composed of lipids. LNPs are typically spherical. The lipid core may, for example, be stabilized by surfactants such as emulsifiers to, for example, prolong shelf life. According to the present invention LNPs comprise nucleic acids such as RNA or DNA, which are encapsulated within the LNP, and can thus be characterized as lipid-nucleic acid particles or as nucleic acid-lipid particles. According to preferred embodiments the LNP of the present invention represents a particle made from different lipids: One or more targeting lipids, and one or more ionizable lipids, and one or more helper lipids, and one or more structural lipids, with or without and one or more stealth lipids.

The term “lipid” as used herein relates to any natural or synthetic lipid. It may accordingly comprise fatty acids and their derivatives including tri-, di-, monoglycerides, and phospholipids, glycerolipids, glycerophospholipids, sphingolipids, saccharolipids, polyketides, sterol lipids and prenol lipids, including DSPE (1,2-distearoyl-sn-glycero-3-phosphoethanolamine), DPPC (1,2-Dipalmitoyl-sn-glyceroyl-3-phosphocholine), DPPE (1,2-Dipalmitoyl-sn-glyceroyl-3-phosphoethanolamine), DMPC (1,2-Dimyristoyl-sn-glyceroyl-3-phosphocholine), DSPC (1,2-Distearoyl-sn-glyceroyl-3-phosphocholine),membrane lipids, and phosphatidylcholine as well as modified versions (modified lipids) thereof. Preferred lipids are DPPC (1,2-Dipalmitoyl-sn-glyceroyl-3-phosphocholine), DPPE (1,2-Dipalmitoyl-sn-glyceroyl-3-phosphoethanolamine), DMPC (1,2-Dimyristoyl-sn-glyceroyl-3-phosphocholine), DSPC (1,2-Distearoyl-sn-glyceroyl-3-phosphocholine) or DSPE (1,2-distearoyl-sn-glycero-3-phosphoethanolamine). The term “modified lipids” is a lipid having one or more modifications. A modification of such a lipid may comprise an acetylation, glycosylation, alkylation, PEGylation, combination with a chelator or a further functionalization such as provision of pH sensitivity, addition of carbon acids, biotin, amines, thioethanl, azide groups etc. The lipids may further be combined with flexibility, elasticity and/or permeability enhancers. Further details are known to the skilled person or can be derived from suitable literature sources such as Benson, 2017, Methods Mol. Biol., 1522: 107-117, Sala et al., 2018, Int J. Pharm. 535 Molecular Cell Biology, 201(1-2), 1-17 or Harayama and Riezman, Nature Reviews, 2018 8, 19, 281-296.

According to the present invention, the LNP comprises a nucleic acid molecule. LNPs comprising a nucleic acid molecule may comprise any suitable number of such molecules, e.g. a single molecule, a number of 1 to 25 molecules, or more than 25 molecules. Further envisaged is that the nucleic acid molecules are of the same type or category, e.g. mRNA, or of different types of type or category, e.g. RNA and DNA. Further envisaged is that the nucleic acids may have the same sequence or comprise different sequences.

In certain embodiments, the LNPs are particles, which comprise a lipid envelope encapsulating said one or more nucleic acid molecules. The term “encapsulating” as used herein means the electrostatic interaction of the nucleic acid with charged lipids of the LNP, causing the mRNA to be positioned inside the LNP and protected from the surrounding environment.

The nucleic acid being comprised in the LNP may have any suitable form or size. For example, the nucleic acid may be DNA or RNA or a derivative of DNA or RNA. In preferred embodiments the nucleic acid is a siRNA, aiRNA, miRNA, ssDNA, dsDNA, ssRNA, short hairpin RNA (shRNA), dsRNA, mRNA, self-amplifying RNA, a plasmid DNA, including plasmids from which an interfering RNA or mRNA is transcribed. Particularly preferred is mRNA.

The term “capable of specifically binding the the receptor Langerin” as used herein means that the LNP comprises a component which allows for an interaction with the receptor Langerin. This leads to the property of the particles to specifically target Langerhans cells (LC), which typically comprise the receptor Langerin at their surface.

The term “Langerin” or “receptor Langerin” as used herein refers to a homotrimeric type II transmembrane receptor and a subtype of C-type lectin receptors located on the surfaces of Langerhans cells, which may also be called “CD207”. The sequence of Langerin as used herein is represented by the wildtype version with the amino acid sequence of SEQ ID NO: 1, or being encoded by the nucleic acid having the nucleotide sequence of SEQ ID NO: 2. The present invention further envisages homologous variants thereof, e.g. amino acid sequence or nucleotide sequence variants having 90%, 91%, 92%, 93%, 94%, 95%, 96%, 97%, 98% or 99% or 99.5% homology with the sequence of SEQ ID NO: 1 or 2. Further envisaged are natural occurring SNP forms of of Langerin, for example, the SNP form V278A (rs741326, NCBI SNP database) with the amino acid sequence represented by SEQ ID NO: 3, encoded by the nucleic acid having the nucleotide sequence of SEQ ID NO: 4. Langerin containing the V278A SNP has a prevalence of 49.9% and has shown similar sugar binding as A278 (Ward et al., 2006. J Biol Chem, 281: 15450-6). Further information can be derived from the NCBI SNP database or a suitable literature source such as Feinberg et al., 2013, J. Biol Chem. 27, 288, 52, 36762-71. The present invention further envisages homologous variants thereof, e.g. amino acid sequence or nucleotide sequence variants having 90%, 91%, 92%, 93%, 94%, 95%, 96%, 97%, 98% or 99% or 99.5% homology with the sequence of SEQ ID NO: 3 or 4. Also envisaged are additional SNP variants such as N288D (rs13383830, NCBI SNP database) with the amino acid sequence represented by SEQ ID NO: 5, encoded by the nucleic acid having the nucleotide sequence of SEQ ID NO: 6; K313I (rs57302492, NCBI SNP database) with the amino acid sequence represented by SEQ ID NO: 7, encoded by the nucleic acid having the nucleotide sequence of SEQ ID NO: 8; and N288D/K313I with the amino acid sequence represented by SEQ ID NO: 9, encoded by the nucleic acid having the nucleotide sequence of SEQ ID NO: 10. The present invention further envisages homologous variants thereof, e.g. amino acid sequence or nucleotide sequence variants having 90%, 91%, 92%, 93%, 94%, 95%, 96%, 97%, 98% or 99% or 99.5% homology with the sequence of any one of SEQ ID NOs: 5 to 10. Further encompassed are codon optimized variants of Langerin. These variants may be adapted to the expression context envisaged, e.g. the codon optimization may be provided for bacterial strains, e.g. E. coli strains, for mammalian cells etc., as would be known to the skilled person. In a specific embodiment, a codon optimized sequence for Langerin containing a StreptagII and TEV site at the C-terminus, which can be used for expression in E. coli, is represented by the nucleotide sequence of SEQ ID NO: 11.

In specific embodiments, “Langerin” may also be a molecule derived from non-human mammals, e.g. mice, monkeys, cows, pigs etc. In a particular embodiment, the present invention thus envisages the use of a mouse Langerin variant with the amino acid sequence represented by SEQ ID NO: 12, encoded by the nucleic acid having the nucleotide sequence of SEQ ID NO: 13. The present invention further envisages homologous variants thereof, e.g. amino acid sequence or nucleotide sequence variants having 90%, 91%, 92%, 93%, 94%, 95%, 96%, 97%, 98% or 99% or 99.5% homology with the sequence of SEQ ID NO: 12 or 13.

The component which allows for the interaction is, according to embodiments of the invention, a targeting lipid. Accordingly, in a specific embodiment, the LNP comprises at least one targeting lipid.

The targeting lipid may preferably be of the general formula (I)

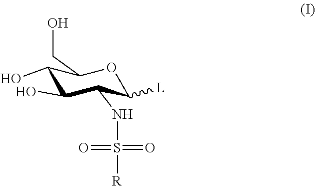

The substituents of the residue R may be any suitable substituent, which is not hindering the ligand in binding to Langerin. Moreover, the substituents can be one or more substituents of either the same type or different types. The substituents of R may have any substitution pattern. In one embodiment, the substituents of the residue R are independently selected from the group consisting of —N(R^{a})(R^{b}), —OR^{a}, —SR^{a}, —C(O)R^{a}, —C(O)OR^{a}, —C(O)N(R^{a})(R^{b}), —N(R^{a})C(O)R^{b}, —N(R^{a})S(O)₂R^{b}, —OS(O)₂R^{a}, halogen, —NO₂, —CN, —NC, —N₃, —NCO, —OCN, —NCS, —SCN, substituted or non-substituted alkyl, alkenyl, alkynyl, aryl and heteroaryl. R^{a} and R^{b} may be independently selected from the group consisting of hydrogen, substituted or non-substituted C₁₋₈ alkyl, C₂₋₈ alkenyl, C₂₋₈ alkynyl, C₃₋₆ cycloalkyl, aryl-C₁₋₅ alkyl, heteroaryl-C₁₋₅ alkyl, aryl, heteroaryl. In a further embodiment, R^{a} and R^{b} may be independently selected from the group consisting of hydrogen, methyl and ethyl.

In a preferred embodiment the substituents of the residue R are independently selected from the group consisting of NH₂, —OH, —OCH₃, —C(O)CH₃, —NHC(O)CH₃, —F, —Cl, —Br, —NO₂, —CN, C₁-C₄ alkyl and phenyl, biphenyl, and naphthyl.

In a further preferred embodiment, the residue R is a substituted phenyl residue. This phenyl residue may be substituted with 5 substituents. More preferably, the phenyl residue may be mono-, di- or trisubstituted. In a most preferred embodiment, the residue R is a monosubstituted phenyl residue, wherein the substituents are selected from the group consisting of —NH₂, —OH, —OCH₃, —C(O)CH₃, C(O)NH₂, —C(O)NHCH₃, —CH₂OH —NHC(O)CH₃, —F, —Cl, —Br, —NO₂, —CN, C₁-C₄ alkyl, naphthyl and phenyl. In a further preferred embodiment, the substituents are in para position to the ligand. In a further preferred embodiment, the substituents in para position and the substituents of the phenyl are independently selected from the group consisting of —NHC(O)CH₃, —CN, —CH₃, —F, —C(O)NH₂, —NH₂, —C(O)NHCH₃, —CH₂OH and phenyl. In a further preferred embodiment, the substituents are in meta position to the ligand.

In another embodiment, R′ may be independently selected from the group consisting of —OR^{a}, and —NHS(O)₂R^{a}, wherein R^{a} is defined as above. In a preferred embodiment, R′ may be selected from the group consisting of —OH, —OCH₃, —OCH₂CH₃, or —NHS(O)₂R^{a}, wherein R^{a} is defined as above. In a more preferred embodiment, —OH or —NHS(O)₂R^{a}, wherein R^{a} is defined as above. In a more preferred embodiment, R′ is —OH, —NHS(O)₂CH₃ or N-tosyl. In the most preferred embodiment, R′ is —OH.

As used herein, the term “alkyl” refers to a straight-chained or branched hydrocarbon group. The hydrocarbon having the indicated number of carbon atoms (e.g., “C1-C8” alkyl refer to an alkyl group having from 1 to 8 carbon atoms). When the number of carbon atoms is not indicated, the alkyl group has from 1 to 100 carbon atoms. Examples of alkyl groups include methyl, ethyl, n-propyl, isopropyl, tert-butyl, and n-pentyl. Alkyl groups may be optionally substituted with one or more substituents.

The term “alkenyl” refers to an unsaturated hydrocarbon chain that may be a straight chain or branched chain, containing 2 to 100 carbon atoms and at least one carbon-carbon double bond. Alkenyl groups may be optionally substituted with one or more substituents.

The term “alkynyl” refers to an unsaturated hydrocarbon chain that may be a straight chain or branched chain, containing the 2 to 100 carbon atoms and at least one carbon-carbon triple bond. Alkynyl groups may be optionally substituted with one or more substituents.

A “substituted” group refers to any substitution of any pattern of that group. This group may be optionally substituted with one or more substituents, wherein the substituents may be of either the same type or different types. Substituents may be selected from the group comprising —N(R^{a})(R^{b}), —OR^{a}, —SR^{a}, —C(O)R^{a}, —C(O)OR^{a}, —C(O)N(R^{a})(R^{b}), —N(R^{a})C(O)R^{b}, —N(R^{a})S(O)₂R^{b}, —OS(O)₂R^{a}, halogen, —NO₂, —CN, —NC, —N₃, —NCO, —OCN, —NCS, —SCN, substituted or non-substituted alkyl, alkenyl, alkynyl, aryl and heteroaryl. Whereas the term “non-substituted” group refers to group, which is a hydrocarbon group.

As used herein, the term “halogen”, “hal” or “halo” means F, Cl, Br or I.

The term “cycloalkyl” refers to a hydrocarbon 3-8 membered monocyclic or 7-14 membered bicyclic ring system or a larger ring system of more than 15 ring members having at least one saturated ring or having at least one non-aromatic ring, wherein the non-aromatic ring may have some degree of unsaturation. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclopentenyl, cyclopentadienyl, cyclohexenyl, cyclohexadienyl, and the like.

The term “aryl” refers to a hydrocarbon monocyclic, bicyclic or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

The term “biaryl” refers to an aromatic ring system containing a substructure that is an assembly of two aromatic rings or aryl groups, if joined by a single bond. The aryl groups may be optionally substituted with one or more substituents. Examples of biaryl groups include biphenyl, binaphthyl and the like.

The term “heteroaryl” refers to a monocyclic, bicyclic or tricyclic aromatic ring system containing at least one heteroatom having carbon atoms (also referred to as ring members) and heteroatom ring members independently selected from N, O, P or S, and derived by removal of one carbon atom from a ring atom of a parent ring system. Examples of heteroaryl groups include furan, thiophene, pyrrole, thiazole, oxazole, pyridine, pyrazine, and the like.

The terms “alkyl cycloalkyl”, “alkyl aryl”, “alkyl biaryl”, and “alkyl heteroaryl” as used herein refer to a saturated or unsaturated hydrocarbon chain that may be a straight chain or branched chain, containing 1 to 8 carbon atoms and may contain carbon-carbon triple bond and/or sp2 hybridized carbons of double bonds bound to cycloalkyl, aryl, biaryl or heteroaryl. The different cyclic structures are defined as above. The cycloalkyl, aryl, biaryl or heteroaryl and/or the alkyl groups may be optionally substituted with one or more substituents. The sp2 or sp carbons of an alkenyl group and an alkynyl group, respectively, may optionally be the point of attachment of the alkenyl or alkynyl groups.

In preferred embodiments, the residue R is independently selected from the group consisting of substituted or non-substituted alkyl, alkenyl, alkynyl, cycloalkyl, C₁-C₈ alkyl cycloalkyl, aryl, C₁-C₈ alkyl aryl, heteroaryl, C₁-C₈ alkyl heteroaryl, biaryl and C₁-C₈ alkyl biaryl. In another embodiment, the residue R is independently selected from the group consisting of substituted or non-substituted alkyl, cycloalkyl, C₁-C₈ alkyl cycloalkyl, aryl, C₁-C₈ alkyl aryl, heteroaryl, C₁-C₈ alkyl heteroaryl, biaryl and C₁-C₈ alkyl biaryl. In a more preferred embodiment, the residue R is independently selected from the group consisting of substituted or non-substituted C₁-C₆ alkyl, C₁-C₆ cycloalkyl, C₁-C₃ alkyl C₁-C₆ cycloalkyl, C₆-C₁₄ aryl, C₁-C₃ alkyl C₆-C₁₄ aryl, heteroaryl, C₁-C₃ alkyl heteroaryl, biaryl and C₁-C₃ alkyl biaryl. In a more preferred embodiment, the residue R is independently selected from the group consisting of substituted or non-substituted cyclohexyl, phenyl, benzyl, biphenyl, pyridyl, or oxazolyl. In another preferred embodiment, the residue R is a substituted or non-substituted phenyl.

In a specific embodiment R is a phenyl or a mono-, di- or trisubstituted phenyl, wherein substituents of the phenyl are independently selected from the group consisting of —NH₂, —OH, —OCH₃, —C(O)CH₃, C(O)NH₂, —C(O)NHCH₃, —CH₂OH —NHC(O)CH₃, —F, —Cl, —Br, —NO₂, —CN, C₁-C₄ alkyl, naphtyl and phenyl.

In preferred embodiments, the targeting lipid is of one of the following formulae:

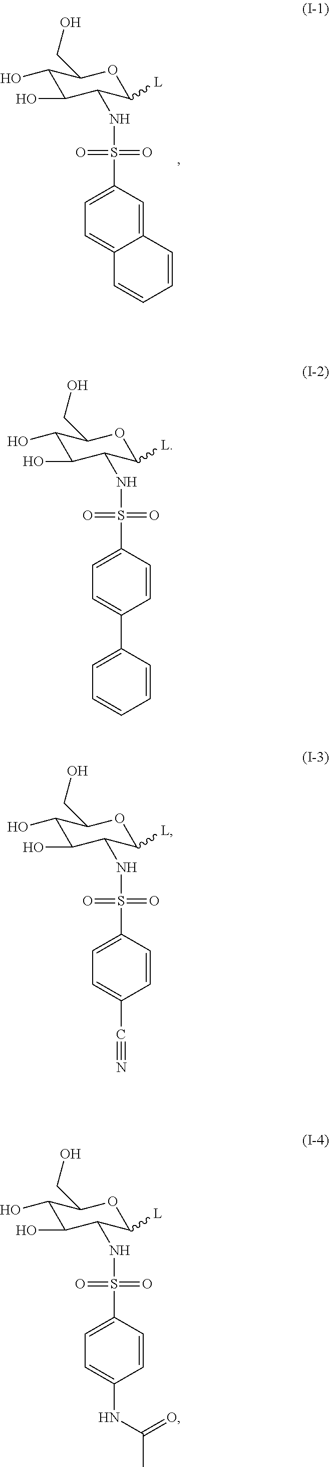

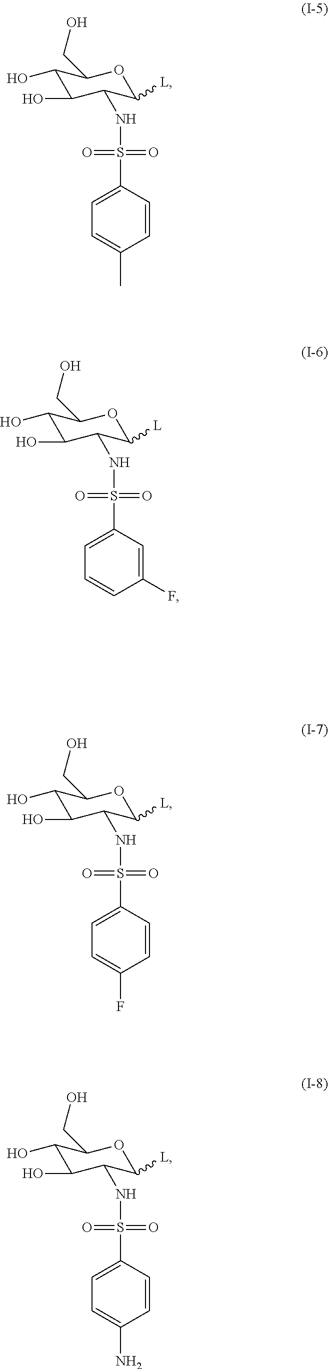

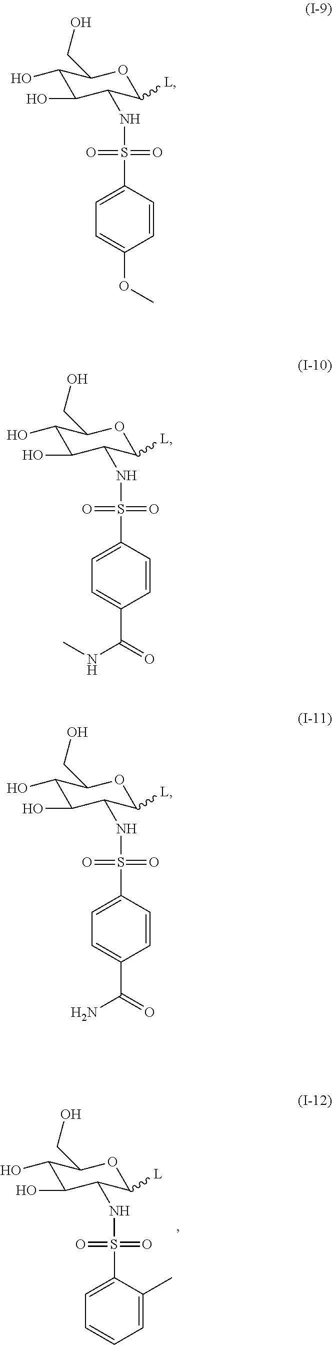

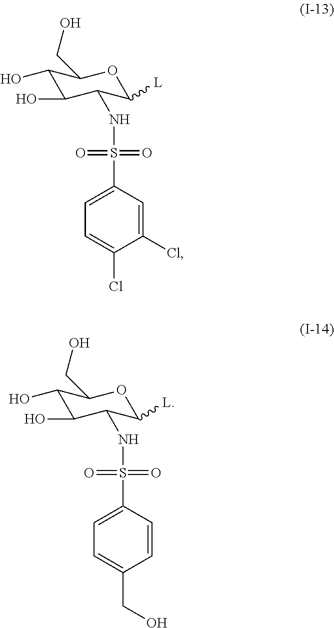

The term “L” as used herein further refer to a component, which is linked to the substituted sugar component of the targeting ligand of the invention as defined herein above.

In a preferred embodiment, said L component may comprise one or more of synthetic polymers or natural polymers or one or more single units of those polymers or a combination thereof. The L component of the present invention is preferably biocompatible and/or biodegradable. The L component may, in certain embodiments, be a synthetic water-soluble polymer that dissolves, disperses or swells in water and, thus, modify the physical properties of aqueous systems in the form of gelation, thickening or emulsification/stabilization. In further embodiments, the synthetic polymer may be a saturated or unsaturated hydrocarbon polymer; a polyamine; a polyamide; a polyester; a polyether, such as polyethylene glycol, polypropylene glycol; a block copolymer or a poloxamer. It is further envisaged that the L component is a polyethylene glycol. The polyethylene glycol linker may have a length of about 0 to 150, more preferably 1 to 100, e.g. 3 to 50 of the (—CH2-CH2-O—) repeating units. Further examples of suitable polymers are polyvinyl pyrrolidone, polyvinyl pyrrolidone-vinyl acetate copolymer, polyvinyl alcohol, polystyrene, polyacrylic acid, polyacylamides, N-(2-hydroxypropyl) methacrylamide and polyoxazoline.

In further specific embodiments the L component is selected from natural polymers such as carbohydrates, modified carbohydrates, peptides, modified peptides, lipids and modified lipids.

The term “carbohydrate” as used herein relates to any natural or synthetic carbohydrate. The term may further comprise trioses, tetroses, pentoses, hexoses and heptoses. Carbohydrates may be aldoses or ketoses. Carbohydrates may be in D- or in L-form. Carbohydrates may comprise one or more monosaccharides or disaccharides. The carbohydrate may form an oligosaccharide including 3 to 9 monosaccharides. The carbohydrate may also be a polysaccharide, which contains more than 9 monosaccharides. The term “monosaccharide” comprises, but is not limited to threose, ribulose, glucose, fructose, galactose, xylose, ribose, arabinose and mannose. Monosaccharides contained in disaccharides, oligosaccharides and polysaccharides may be linked to each other in any configuration. The term “disaccharide” comprises, but is not limited to sucrose, lactose, and maltose. The term “oligosaccharide” comprises but is not limited to maltodextrins and cellodextrins. The term “polysaccharide” comprises but is not limited to starch, cellulose, and chitin. Natural carbohydrates comprise natural monosaccharides. Synthetic carbohydrates may comprise D- and L-, modified, synthetic, unusual monosaccharides and monosaccharides derivatives. A “monosaccharide derivative” also called “modified carbohydrates” includes monosaccharides having substitutions or modifications by covalent attachment of a parent monosaccharides, such as, e.g., by alkylation, acetylation, phosphorylation, and the like. Further included within the definition of “monosaccharides derivative” are, for example, one or more analogs of a monosaccharide with substituted linkages, as well as other modifications known in the art. Preferred carbohydrates are mannose, glucose, fucose or xylose. In certain embodiments also inositols may be used.

It is particularly preferred that L is a lipid or a modified lipid. Examples of preferred L components include a phospholipid, 1,2-distearoyl-sn-glycero-3-phosphoethanolamine (DSPE), oxyglutaryl aminopropyl polyethyleneglycol-carbamyl distearoylphosphatidyl-ethanolamine (DSPE-PEG), a membrane lipid, or a modified phosphatidylcholine.

In a further particularly preferred embodiment L is of the following formula (II):

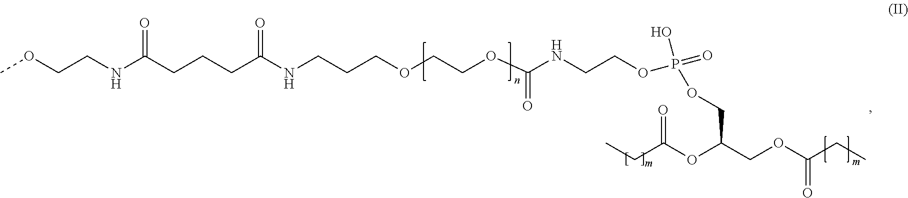

wherein n is an integer from 0 to 150 and m is an integer from 1 to 30.

In specific embodiments of formula (II) n may be an integer of 0, 5, 10, 15 20, 25, 30, 35, 40, 45, 50, 55, 60, 65, 70, 75, 80, 85, 90, 95, 100, 105, 110, 115, 120, 125, 130, 135, 140, 145 or 150, or any integer between the mentioned integers. In further specific embodiments m may be an integer of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30.

In a further particularly preferred embodiment the targeting lipid is of the following formula (III)

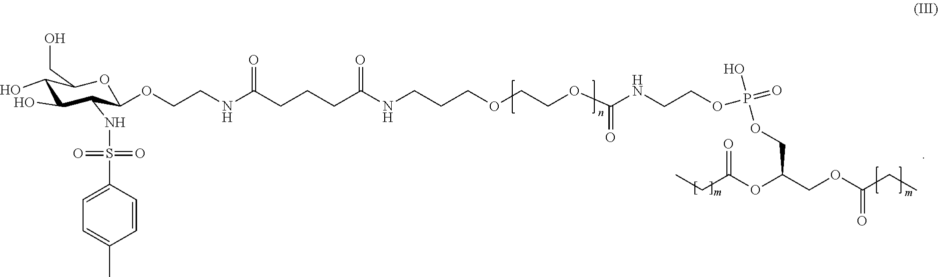

wherein n is an integer from 0 to 150 and m is an integer from 1 to 30.

In specific embodiments of formula (III) n may be an integer of 0, 5, 10, 15 20, 25, 30, 35, 40, 45, 50, 55, 60, 65, 70, 75, 80, 85, 90, 95, 100, 105, 110, 115, 120, 125, 130, 135, 140, 145 or 150, or any integer between the mentioned integers. In further specific embodiments m may be an integer of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30.

The invention further envisages, in a specific embodiment, that the LNP may comprise at least one stealth lipid. The term “stealth lipid” as used herein refers to a molecule that improves LNP stability and minimise LNP clearance from the body. Stealth lipids typically comprise a covalently bound polyethylene glycol chain of a defined length to a lipid moiety. Further details can be derived from suitable literature sources such as Nanoscale, 2021, 13, 10748-10764.

It is preferred that the stealth lipid is DSPE-PEG (N-(Methylpolyoxyethylene oxycarbonyl)-1,2-distearoyl-sn-glycero-3-phosphoethanolamine), also known as CAS 247925-28-6, DMG-PEG (1,2-dimyristoyl-rac-glycero-3-methoxypolyethylene glycol), also known as CAS 160743-62-4, or DSG-PEG (1,2-Distearoyl-rac-glycero-3-methylpolyoxyethylene), also known as CAS 308805-39-2.

The term “CAS” refers to unique numeric identifiers assigned by the Chemical Abstracts Service (CAS), a division of the American Chemical Society, to chemical compounds. Each CAS Number is a distinct and universally recognized numerical identifier associated with a specific chemical substance, facilitating precise and unambiguous identification, retrieval, and categorization of chemical compounds, e.g. as mentioned in the patent application.

It is particularly preferred that the stealth lipid is DSPE-PEG2000 N-(Carbonyl(methoxypolyethylene glycol)]-1,2-distearoyl-sn-glycero-3-phosphoethanolamine, also known as CAS 247925-28-6.

The LNP may comprise one stealth lipid or more than one stealth lipid. The LNP may further comprise one type of stealth lipid, e.g. selected from DSPE-PEG (N-(Methylpolyoxyethylene oxycarbonyl)-1,2-distearoyl-sn-glycero-3-phosphoethanolamine), DMG-PEG (1,2-dimyristoyl-rac-glycero-3-methoxypolyethylene glycol) or DSG-PEG (1,2-Distearoyl-rac-glycero-3-methylpolyoxyethylene) and DSPE-PEG2000 N-[Carbonyl(methoxypolyethylene glycol)]-1,2-distearoyl-sn-glycero-3-phosphoethanolamine, or more than one type of stealth lipid, e.g. selected from DSPE-PEG (N-(Methylpolyoxyethylene oxycarbonyl)-1,2-distearoyl-sn-glycero-3-phosphoethanolamine), DMG-PEG (1,2-dimyristoyl-rac-glycero-3-methoxypolyethylene glycol) or DSG-PEG (1,2-Distearoyl-rac-glycero-3-methylpolyoxyethylene) or DSPE-PEG2000 N-[Carbonyl(methoxypolyethylene glycol)]-1,2-distearoyl-sn-glycero-3-phosphoethanolamine. Accordingly, the pharmaceutical composition may comprise a combination of 2 or more different stealth lipids, e.g. selected from DSPE-PEG (N-(Methylpolyoxyethylene oxycarbonyl)-1,2-distearoyl-sn-glycero-3-phosphoethanolamine), DMG-PEG (1,2-dimyristoyl-rac-glycero-3-methoxypolyethylene glycol) or DSG-PEG (1,2-Distearoyl-rac-glycero-3-methylpolyoxyethylene) and DSPE-PEG2000 N-[Carbonyl(methoxypolyethylene glycol)]-1,2-distearoyl-sn-glycero-3-phosphoethanolamine.

The invention further envisages that the LNP comprises at least one helper lipid. The term “helper lipid” as used herein refers to a molecule that effects the LNP's membrane properties. Helper lipids commonly have phospholipid structure, with a hydrophilic head group and one or more hydrophobic tail groups.

It is preferred that the helper lipid is DSPC (1,2-Distearoyl-sn-glycero-3-phosphocholine), also known as CAS 816-94-4, DSPE (1,2-Distearoyl-sn-glycero-3-phosphoethanolamine), also known as CAS 1069-79-0, or DOPE (1,2-Dioleoyl-sn-glycero-3-phosphoethanolamine), also known as CAS 4004-05-1.

The LNP may comprise one helper lipid or more than one helper lipid. The LNP may further comprise one type of helper lipid, e.g. selected from DSPC (1,2-Distearoyl-sn-glycero-3-phosphocholine), also known as CAS 816-94-4, DSPE (1,2-Distearoyl-sn-glycero-3-phosphoethanolamine), also known as CAS 1069-79-0 or DOPE (1,2-Dioleoyl-sn-glycero-3-phosphoethanolamine), also known as CAS 4004-05-1, or more than one type of helper lipid, e.g. selected from DSPC (1,2-Distearoyl-sn-glycero-3-phosphocholine), also known as CAS 816-94-4, DSPE (1,2-Distearoyl-sn-glycero-3-phosphoethanolamine), also known as CAS 1069-79-0 and DOPE (1,2-Dioleoyl-sn-glycero-3-phosphoethanolamine) also known as CAS 4004-05-1. Accordingly, the LNP may comprise a combination of 2 or more different helper lipids, e.g. selected from DSPC (1,2-Distearoyl-sn-glycero-3-phosphocholine), also known as CAS 816-94-4, DSPE (1,2-Distearoyl-sn-glycero-3-phosphoethanolamine), also known as CAS 1069-79-0, and DOPE (1,2-Dioleoyl-sn-glycero-3-phosphoethanolamine), also known as CAS 4004-05-1.

The invention further envisages that the LNP comprises at least one structural lipid. The term “structural lipid” as used herein accordingly refers to a molecule that effects the LNP's structural membrane properties, such as rigidity and fluidity. Structural lipids typically comprise a steroid backbone and one or more hydrophobic moieties.

It is preferred that the structural lipid is cholesterol or a cholesterol analog.

The LNP may comprise one structural lipid or more than one structural lipid. The LNP may further comprise one type of structural lipid, e.g. selected from cholesterol, also known as CAS 57-88-5, and cholesterol analogs or more than one type of structural lipid, e.g. selected from cholesterol and cholesterol analogs. Accordingly, the LNP may comprise a combination of 2 or more different structural lipids, e.g. selected from cholesterol or cholesterol analogs.

The invention further envisages that the LNP comprises at least one ionizable lipid. The term “ionizable lipid” as used herein accordingly refers to a compound or molecule that bears a group or atom, which is charged at a certain pH and uncharged at another pH of its environment.

It is preferred that the ionizable lipid is DLin-MC3-DMA ((6Z,9Z,28Z,31Z)-Heptatriaconta-6,9,28,31-tetraen-19-yl 4-(dimethylamino) butanoate), also known as CAS 2089251-47-6, SM-102 (8-[(2-hydroxyethyl)[6-oxo-6-(undecyloxy)hexyl]amino]-octanoic acid, 1-octylnonyl ester), also known as CAS 2089251-47-6, ALC-0315 (2-hexyl-decanoic acid, 1,1′-[[(4-hydroxybutyl) imino]di-6,1-hexanediyl]ester), also known as CAS 2036272-55-4, or DODMA (1,2-Dioleyloxy-3-dimethylamino-propane), also known as CAS 104162-47-2.

The LNP may comprise one ionizable lipid or more than one ionizable lipid. The LNP may further comprise one type of ionizable lipid, e.g. selected from DLin-MC3-DMA ((6Z,9Z,28Z,31Z)-Heptatriaconta-6,9,28,31-tetraen-19-yl 4-(dimethylamino) butanoate), also known as CAS 2089251-47-6, SM-102 (8-[(2-hydroxyethyl)[6-oxo-6-(undecyloxy)hexyl]amino]-octanoic acid, 1-octylnonyl ester), also known as CAS 2089251-47-6, ALC-0315 (2-hexyl-decanoic acid, 1,1′-[((4-hydroxybutyl) imino]di-6,1-hexanediyl]ester), also known as CAS 2036272-55-4, and DODMA (1,2-Dioleyloxy-3-dimethylamino-propane), also known as CAS 104162-47-2 or more than one type of ionizable lipid, e.g. selected from DLin-MC3-DMA ((6Z,9Z,28Z,31Z)-Heptatriaconta-6,9,28,31-tetraen-19-yl 4-(dimethylamino) butanoate), also known as CAS 2089251-47-6, SM-102 (8-[(2-hydroxyethyl)[6-oxo-6-(undecyloxy)hexyl]amino]-octanoic acid, 1-octylnonyl ester) also known as CAS 2089251-47-6, ALC-0315 (2-hexyl-decanoic acid, 1,1′-[[(4-hydroxybutyl) iminodi-6,1-hexanediyl]ester), also known as CAS 2036272-55-4, and DODMA (1,2-Dioleyloxy-3-dimethylamino-propane), also known as CAS 104162-47-2. Accordingly, the LNP may comprise a combination of 2 or more different ionizable lipids, e.g. selected from DLin-MC3-DMA ((6Z,9Z,28Z,31Z)-Heptatriaconta-6,9,28,31-tetraen-19-yl 4-(dimethylamino) butanoate), also known as CAS 2089251-47-6, SM-102 (8-[(2-hydroxyethyl)(6-oxo-6-(undecyloxy)hexyl]amino]-octanoic acid, 1-octylnonyl ester), also known as CAS 2089251-47-6, ALC-0315 (2-hexyl-decanoic acid, 1,1′-[[(4-hydroxybutyl) imino]di-6,1-hexanediyl]ester), also known as CAS 2036272-55-4, and DODMA (1,2-Dioleyloxy-3-dimethylamino-propane), also known as CAS 104162-47-2.

In a specific embodiment, the LNP comprises a targeting lipid and a combination of different lipids, such as one or more helper lipids, one or more structural lipids, one or more ionizable lipids and one or more stealth lipids. It is preferred that the LNP comprises a targeting lipid, a helper lipid, a structural lipid and an ionizable lipid. In certain embodiments, the presence of a stealth lipid may be optionally envisaged.

According to certain embodiments of the present invention the pharmaceutical composition may comprise one or more LNPs. For example, the pharmaceutical composition may comprise about 1, 2, 3, 4, 5, 6, 7, 8, 9, 10, 20, 30, 40, 50, 60, 70, 80, 90, 100, 150, 200, 300, 400, 500, 600, 700, 800, 900, 1000 or more than 1000 LNPs, or any value in between the indicated values, in a formulation. These LNPs may be of the same types, size or comprise the same nucleic acid, or may be of different types, different sizes or comprise different nucleic acids, or any combination of the above, e.g. different types, but same size and Identical nucleic acids, or same type, but different sizes and different nucleic acids etc.

In preferred embodiments the LNP has a size of about 30 to 250 nm. The LNP may, for example, have a size of 30, 40, 50, 60, 70, 80, 90, 100, 110, 120, 130, 140, 150, 160, 170, 180, 190, 200, 210, 220, 230, 240 or 250 nm or any size in between the mentioned sizes. The size may depend on the amount and/or type of lipids or modified lipids included, or the formulation process parameters such as the flow rate ratio. It is preferred that the LNP has a size of 80 to 140 nm. Further envisaged are combinations of different sized LNPs in a pharmaceutical composition. For example, a pharmaceutical composition may comprise a group of LNPs of the same size or, alternatively, or different sizes.

In preferred embodiments the pharmaceutical composition as defined above comprises a solvent or a combination of a solvent with a further compound. The solvent may be any suitable solvent known to the skilled person. It is preferred that the solvent is H₂O, an aqueous sucrose solution, a phosphate buffered saline, an aqueous sodium chloride solution, tricine buffer, or HEPES buffer.

The “further compound” may be, for example, DMSO, propylene glycol, or oleic acid. It is preferred that the further compound is propylene glycol. It is particularly preferred that propylene glycol is provided in a concentration of up to 80%, e.g. in a concentration of 5%, 10%, 15%, 20%, 25%, 30%, 35%, 40%, 45%, 50%, 55%, 60%, 65%, 70%, 75% or 80% or at any value in between the mentioned values. It is particularly preferred that the concentration of propylene glycol is about 80%. It is further preferred to include propylene glycol for topical administration since it can be used to increase the permeability of the underlying tissue, e.g. the skin.

In further embodiments, the pharmaceutical composition as defined herein above is provided as a dry composition. The term “dry composition” means that the composition does not comprise a solvent or aqueous solution. It is particularly preferred that the pharmaceutical composition is provided as powder composition. A dry composition may, for example, be produced by lyophilizing a pharmaceutical composition comprising one or more LNPs or groups of LNPs etc.

In a set of specific embodiments the amount of the targeting lipids as defined above in the LNP is about 0.01 mol % to 5 mol %. For example, the amount of targeting lipids in the LNP may be about 0.01 mol %, 0.05 mol %, 0.1 mol %, 0.5 mol %, 1 mol %, 2 mol %, 3 mol %, 4 mol % or 5 mol % or any value in between the mentioned values. It is preferred that the amount of targeting lipids in the LNP is about 0.1 mol % to 3 mol %. For example, the amount of targeting lipids in the LNP may be about 0.1 mol %, 0.5 mol %, 1 mol %, 2 mol % or 3 mol % or any value in between the mentioned values. It is particularly preferred that the amount of targeting lipids in the LNP is 0.25 mol % to 2 mol %, e.g. 0.25 mol %, 0.5 mol %, 0.75 mol %, 1 mol %, 1.25 mol %, 1.5 mol %, 1.75 mol % or 2 mol %.

The term “mol %” in the context of the above and following embodiments is meant to be in reference to the total amount of lipids. The total amount of lipids in the LNP according to the present invention is 100 mol %.

In a further set of specific embodiments the amount of the stealth lipids lipids as defined above in the LNP is about 0 mol % to 5 mol %. For example, the amount of stealth lipids in the LNP may be about 0.01 mol %, 0.05 mol %, 0.1 mol %, 0.5 mol %, 1 mol %, 2 mol %, 3 mol %, 4 mol % or 5 mol % or any value in between the mentioned values. In certain embodiments, there may be no stealth lipid present, i.e. its amount being 0 mol %. It is preferred that the amount of stealth lipids in the LNP is about 0.1 mol % to 2 mol %, e.g. 0.5 mol %, 0.75 mol %, 1 mol %, 1.25 mol %, 1.5 mol %, 1.75 mol % or 2 mol % or any value in between the mentioned values.

In a further set of specific embodiments the amount of the helper lipids as defined above in the LNP is about 5 mol % to 15 mol %. For example, the amount of helper lipids in the LNP may be about 5 mol %, 6 mol %, 7 mol %, 8 mol %, 9 mol %, 10 mol %, 11 mol %, 12 mol %, 13 mol %, 14 mol % or 15 mol % or any value in between the mentioned values. It is preferred that the amount of helper lipids in the LNP is about 10 mol %.

In a further set of specific embodiments the amount of the structural lipids as defined above in the LNP is about 20 mol % to 60 mol %. For example, the amount of structural lipids in the LNP may be about 20 mol %, 25 mol %, 30 mol %, 35 mol %, 40 mol %, 45 mol %, 50 mol %, 55 mol % or 60 mol % or any value in between the mentioned values. It is preferred that the amount of structural lipids in the LNP is about 35 mol % to 40 mol %, e.g. about 35 mol %, 36 mol %, 37 mol %, 38 mol %, 39 mol % or 40 mol % or any value in between the mentioned values.

In a further set of specific embodiments the amount of the ionizable lipids as defined above in the LNP is about 10 mol % to 70 mol %. For example, the amount of ionizable lipids in the LNP may be about 10 mol %, 20 mol %, 30 mol %, 40 mol %, 50 mol %, 60 mol %, or 70 mol % or any value in between the mentioned values. It is preferred that the amount of ionizable lipids in the LNP is about 15 mol % to 60 mol %. It is particularly preferred that the amount of ionizable lipids in the LNP about 20 mol % to 55 mol %, e.g. 20 mol %, 25 mol %, 30 mol %, 35 mol %, 40 mol %, 45 mol %, 50 mol % or 55 mol % or any value in between the mentioned values.

In certain embodiments, the amount of stealth lipids is present in the LNP in addition to the amount of targeting lipids. In further embodiments, the amount of stealth lipids is present in addition to the amount of targeting lipids and the amount of helper lipids and structural lipids and ionizable lipids as defined herein. In the LNP the overall amount of lipids is 100 mol %.

In further embodiments, the amount of helper lipids is present in the LNP in addition to the amount of targeting lipids. In further embodiments, the amount of helper lipids is present in addition to the amount of targeting lipids and structural lipids and ionizable lipids and optionally the amount of stealth lipids as defined herein. In the LNP the overall amount of lipids is 100 mol %.

In further embodiments, the amount of structural lipids is present in the LNP in addition to the amount of targeting lipids. In further embodiments, the amount of structural lipids is present in addition to the amount of targeting lipids and the amount of stealth lipids and helper lipids and ionizable lipids and optionally the amount of stealth lipids as defined herein. In the LNP the overall amount of lipids is 100 mol %.

In further embodiments, the amount of ionizable lipids is present in the LNP in addition to the amount of targeting lipids. In further embodiments, the amount of ionizable lipids is present in addition to the amount of targeting lipids and helper lipids and structural lipids and optionally the amount of stealth lipids as defined herein. In the LNP the overall amount of lipids is 100 mol %.

In a particularly preferred embodiment, the LNP comprises of a targeting lipid with a molar ratio of 0.25 mol % to 2 mol %, and a helper lipid with a molar ratio of 5 mol % to 15 mol %, and a structural lipid with a molar ratio of 35 mol % to 40 mol %, and an ionizable lipid with a molar ratio of 20 mol % to 55 mol %, and a stealth lipid with a molar ratio of 0 mol % to 0.25 mol %. In this embodiment the overall amount of lipids in the LNP is 100 mol %.

In further embodiments, the LNP comprises of a targeting lipid as depicted in formula (III) with a molar ratio of 0.25 mol % to 2 mol %, and DSPC (as the helper lipid) with a molar ratio of 5 mol % to 15 mol %, and cholesterol (as the structural lipid) with a molar ratio of 35 mol % to 40 mol %, and DLin-MC3-DMA (as the ionizable lipid) with a molar ratio of 20 mol % to 55 mol %, and PEG2000-DSPE (as the stealth lipid) with a molar ratio of 0 mol % to 0.25 mol %. In this embodiment the overall amount of lipids in the LNP is 100 mol %.

In further embodiments, the LNP comprises of a targeting lipid as depicted in formula (III) with a molar ratio of 0.25 mol % to 2 mol %, and DSPC (as the helper lipid) with a molar ratio of 5 mol % to 15 mol %, and cholesterol (as the structural lipid) with a molar ratio of 35 mol % to 40 mol %, and SM-102 (as the ionizable lipid) with a molar ratio of 20 mol % to 55 mol %, and PEG2000-DSPE (as the stealth lipid) with a molar ratio of 0 mol % to 0.25 mol %. In this embodiment the overall amount of lipids in the LNP is 100 mol %.

In further embodiments, the LNP comprises of a targeting lipid as depicted in formula (III) with a molar ratio of 0.25 mol % to 2 mol %, and DSPC (as the helper lipid) with a molar ratio of 5 mol % to 15 mol %, and cholesterol (as the structural lipid) with a molar ratio of 35 mol % to 40 mol %, and ALC-0315 (as the ionizable lipid) with a molar ratio of 20 mol % to 55 mol %, and PEG2000-DSPE (as the stealth lipid) with a molar ratio of 0 mol % to 0.25 mol %. In this embodiment the overall amount of lipids in the LNP is 100 mol %.

In further embodiments, the LNP comprises of a targeting lipid as depicted in formula (III) with a molar ratio of 0.25 mol % to 2 mol % and DSPC (as the helper lipid) with a molar ratio of 5 mol % to 15 mol %, and cholesterol (as the structural lipid) with a molar ratio of 35 mol % to 40 mol %, and DODMA (as the ionizable lipid) with a molar ratio of 20 mol % to 55 mol %, and PEG2000-DSPE (as the stealth lipid) with a molar ratio of 0 mol % to 0.25 mol %. In this embodiment the overall amount of lipids in the LNP is 100 mol %.

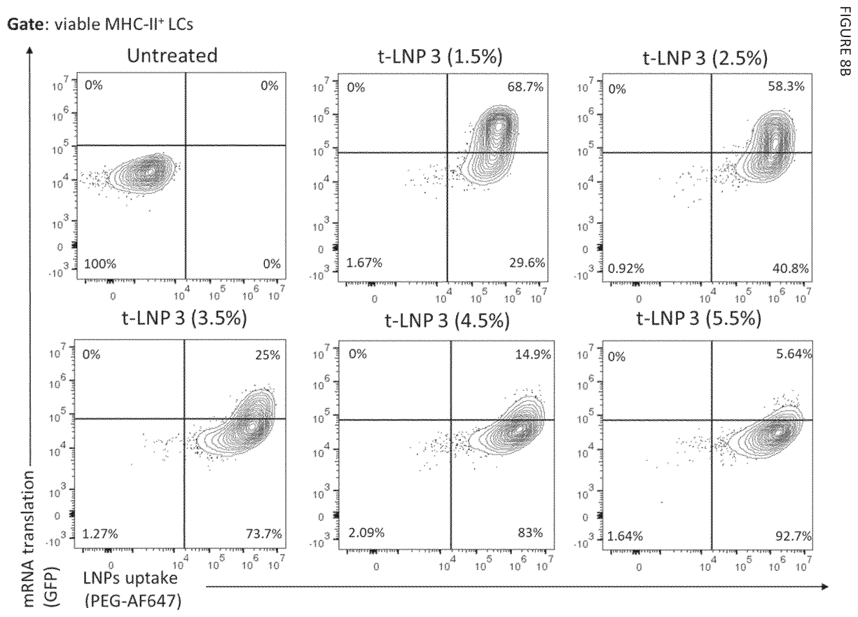

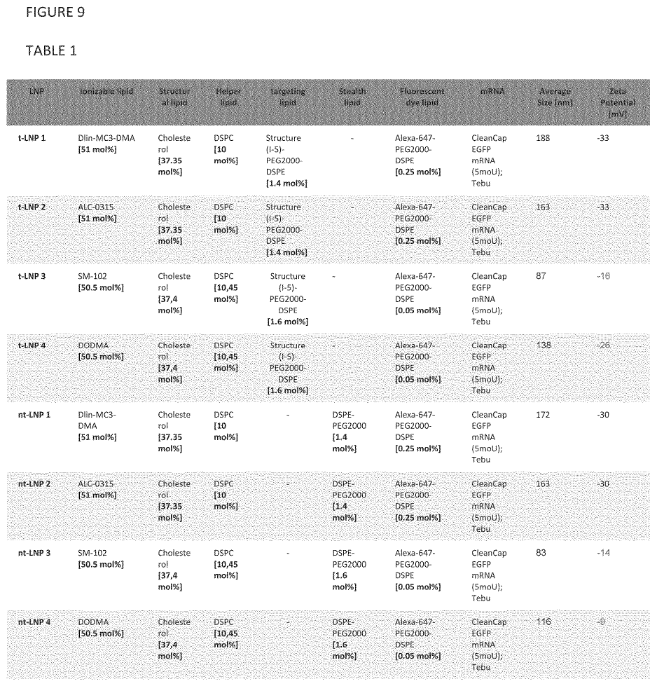

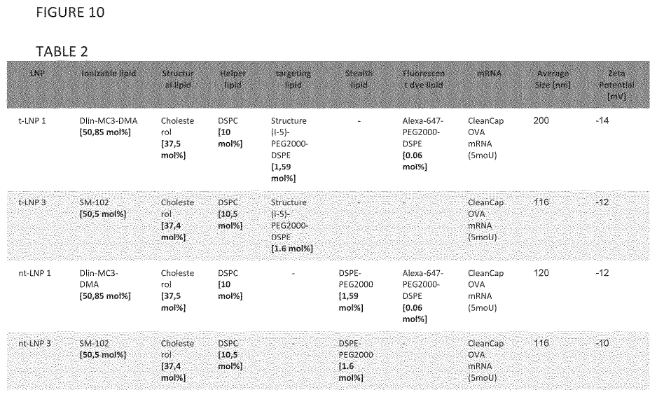

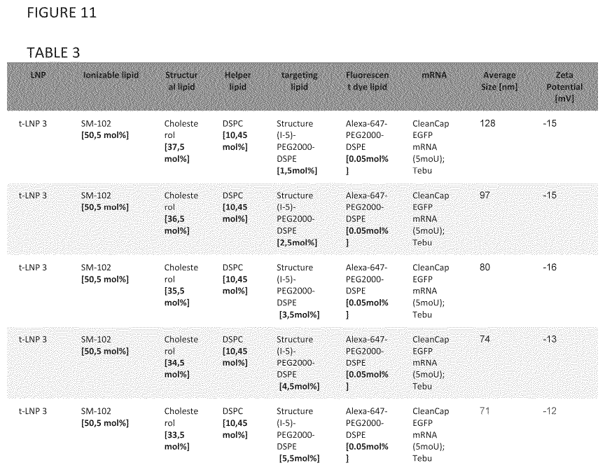

In further embodiments, a specific combination of lipids and their molar ratios can also be derived from Table 1 (FIG. 9), Table 2 (FIG. 10) or Table 3 (FIG. 11), whose values concerning said combinations and molar ratios are incorporated by reference.

For example, the LNP comprises a combination of lipids and their molar ratios as indicated for t-LNP 1, t-LNP 2, t-LNP 3, t-LNP 4 in Table 1 (FIG. 9).

In further embodiments, the LNP comprises of a combination of lipids and their molar ratios as given for t-LNP 1, t-LNP 3 in Table 2 (FIG. 10).

In further embodiments, the LNP comprises of a combination of lipids and their molar ratios as given for t-LNP 3 in Table 3 (FIG. 11).

In preferred embodiments the nucleic acid encapsulated in the LNP is an mRNA which encodes for a pharmaceutically or immunologically active compound.

The term “pharmaceutically active compound” as used herein relates to any suitable substance, drug, cellular component, tissue portion, or active pharmaceutical ingredient (API) composed of amino acids known to the skilled person. These compounds comprise, for example, therapeutic proteins, cytotoxic proteins, or peptide or protein inhibitors. In a particularly preferred embodiment, the pharmaceutically active compound is a modulator of cellular function. The term “modulator of cellular function” as used herein relates to any peptide or polypeptide which has an inhibitory or stimulatory effect on physiological functions, preferably on protein function like enzymatic functions. The inhibition may, for example be a decrease in the activity of an enzyme, as compared to the activity of the enzyme in the absence of the inhibitor. In some embodiments, the term “inhibit” thus means a decrease in enzyme activity of at least about 5%, at least about 10%, at least about 20%, at least about 25%, at least about 50%, at least about 60%, at least about 70%, at least about 80%, at least about 90%, or at least about 95%. In other embodiments, “inhibit” means a decrease in enzyme activity of about 5% to about 25%, about 25% to about 50%, about 50% to about 75%, or about 75% to 100%. Also envisaged is a decrease in enzyme activity of about 95% to 100%, e.g., a decrease in activity of 95%, 96%, 97%, 98%, 99% or 100%. Such decreases can be measured using any suitable method or assay known to a person skilled in the art.

Examples of such inhibitors are inhibitors of apoptosis such as proteins of the Bi-2 family such as Bcl-2, Bcl-XL, or Bcl-w, or crmA (cytotoxin response modifier A), which may be used to inhibit caspase 1, 6 and 8. Also contemplated is the use of IAPs (inhibitors of apoptosis proteins), including Cp-IAP, Op-IAP, XIAP, cIAP1, C-IAP2, NAIP, Livin and Survivin.

term “immunologically active compound” as used herein relates to any compound composed of amino acids, which is capable eliciting an immunological reaction in the body. In further embodiments, it may alternatively be capable of immunomodulation. Also envisaged is that the immunologically active compound is an immunological tolerance inducer.

The term “compound capable of eliciting an immunological reaction in the body” as used herein relates to any substance or part of a substance composed of amino acids which is recognized by elements of an animal's, preferably a mammal's, most preferably the human immune system and which leads to an activation of the innate immune system or the adaptive immune system.

Dendritic cells (DCs) are key cellular components of the innate immune system. These cells recognize and take up antigens both originating from outside the organism (also named “non-self antigens”) or from inside the organism (also named “self-antigens”). Non-self antigens derive for instance from microbes, such as bacteria, viruses, parasites or other foreign substances. Self-antigens instead are antigens derived from the same organism and may include cancer antigens. Upon uptake, DCs process the antigens and present them to lymphocytes in a process defined as antigen presentation. Upon antigen presentation and through upregulation of costimulatory molecules and proinflammatory cytokines DCs instruct the lymphocytes to initiate an antigen-specific pro-inflammatory immune response. On the contrary, antigen presentation associated with the upregulation of co-inhibitory molecules and anti-inflammatory cytokines by DCs initiate an antigen-specific tolerogenic response. The DC-T cell interaction leads to Th1/Th2/Th17/Treg instruction, or T cell deletion and anergy, or cytotoxic T-lymphocyte (CTL, T-killer cell) activation. Different DC subsets exist across the body and serve to initiate pro-inflammatory or tolerogenic responses or both. One DC subset capable of initiating both responses are the Langerhans cells (LCs). LCs are the only DCs present in the epidermis. Here LCs take up both self- and non-self antigens, discriminate if such antigens are of pathogenic origin, process and present the antigens on MHC-Class I and II molecules. Finally, LCs migrate to lymph nodes to present the cognate antigen to T cells.

The adaptive immune system, on the other hand, is primarily based on the activity of specialized lymphocytes, i.e. B cells and T cells. The B cells are typically involved in humoral immune responses, whereas T cells are involved in cell-mediated immune responses. Both, B- and T cells express antigen receptors (B cell receptor (BCR) or T cell receptor (TCR), respectively) which recognize peptides derived from specific antigens. Two main types of T cells can be classified, into cytotoxic T cells (CTLs), also known as CD8⁺ T cells or killer T cells, and helper T cells, also known as CD4⁺ T cells, comprising also regulatory T cells. Antigens inside a cell are typically bound to class I MHC molecules (MHC 1) and brought to the surface of the cell by MHCI, where they can be recognized by the T cell. If the TCR is specific for that antigen, it binds to the complex of MHC I and the antigen, and the T cell destroys the presenting cell. MHC I molecules can be found on the surface of all nucleated cells. MHC I molecules typically bind peptides generated mainly from degradation of cytosolic proteins by the proteasome. The MHC I:peptide complex is subsequently inserted via endoplasmic reticulum into the external plasma membrane of the cell. The epitope peptide is bound on extracellular parts of the MHC I molecule. Thus, the function of the class I MHC molecules is believed to mainly display intracellular proteins to cytotoxic T cells (CTLs). In addition, class I MHC can also present peptides generated from exogenous proteins via cross-presentation, which is the ability of certain antigen-presenting cells, e.g. DCs, to take up, process and present extracellular antigens with MHC class I molecules to cytotoxic CD8⁺ T cells. Cross-priming, the result of this process, describes the stimulation of naive cytotoxic CD8⁺ T cells into activated cytotoxic CD8⁺ T cells. This process may lead to immunity against tumors and viruses. Cross presentation may also advantageously be for the induction of cytotoxic immunity, e.g. by vaccination with protein antigens, for example, tumour vaccination as described herein. MHC I molecules typically bind peptides that are 8-10 amino acid in length.

Helper T cells (also known as CD4⁺ T cells) and regulatory T cells (also known as Treg cells or suppressor T cells), on the other hand, recognize antigens which are coupled to class II MHC molecules. MHC II molecules are typically only found on antigen presenting cells (APCs), such as dendritic cells, mononuclear phagocytes, thymic epithelial cells or B cells. The loading of MHC class II molecules typically occurs in lysosomal compartments. For example, extracellular proteins may be endocytosed, digested in lysosomes and epitopic peptide fragments can be bound to MHC II molecules. Typically, MHC II molecules present antigens of a length of between about 15 to 24 amino acids.

In accordance with the present invention, any of the above-described activities may be elicited by a suitable compound encoded by an mRNA molecule encapsulated in an LNP as defined above, e.g. an antigen, or an epitope. The length of the antigen, its cell compartment localisation etc. may modulate the presentation on MHC I or MHC II molecules and thus also modulate the activation of certain branches of the immune system. For cancer and viral therapy. It is particularly preferred that a close interaction of the innate and adaptive immune system be elicited, e.g. via activation of the DCs. For allergy and autoimmune disease therapies, it is particularly preferred that activation of the DCs is minimized to induce a tolerogenic effect. Further details may be derived from suitable literature sources such as Ortner et al., Oncoimmunology, 2017, 6, 2, e1260215; Watt et al., 2008, J Immunol., 181, 8, 5323-30 or Walzer et al., 2005, Blood, 106, 7, 2252-8; Krienke et al., 2021, Science, 371, 145.

In certain embodiments of the present invention said mRNA encodes for any one of: a cancer antigen or epitope; an autoimmune disease antigen or epitope; a bacterial antigen or epitope; a viral antigen or epitope; a parasitic antigen or epitope; or an allergen, or an epitope of an allergen.

The term “cancer antigen” as used herein relates to antigenic substances composed of amino acids produced in tumor cells. The term may thus typically also include cellular antigens. These antigenic substances may alternatively also be named “tumor antigens”. These antigens typically trigger immune responses in the host. Without wishing to be bound by theory, it is currently believed that normal proteins in a body (i.e. proteins produced by the host itself) are not antigenic due to self-tolerance of the immune system, a concept in which autoreactive lymphocytes are deleted before they develop into fully immunocompetent cells. Other proteins which are not exposed to the immune system may trigger an immune response. This may include proteins which are sequestered from the immune system, proteins which are normally produced in very small quantities (but are produced, for example, in a much higher amount in cancerous cells), or proteins which are produced typically only during certain stages of the cellular/organism's development, or proteins whose structure or function is changed due to the presence of a mutation. Accordingly, the cancer antigens may be classified into different groups, e.g. as products of mutated oncogenes and tumor suppressor genes, products of other mutated genes such as (i) overexpressed or aberrantly expressed cellular proteins, (ii) cancer antigens produced by oncogenic viruses, (iii) oncofetal antigens, (iv) altered cell surface glycolipids and glycoproteins, and (v) cell type-specific differentiation antigens. In further specific embodiments, the antigen may be a tumor-specific antigen, which is an antigen that is produced by a mutation to a gene coding for a protein whose abnormal production is the cause of a cancer or tumor. An envisaged example of such tumor-specific antigens is an abnormal form of p53 or ras. Alternatively, if a mutation is unrelated to the tumor development, but lead to the production of an abnormal protein which is associated with the cancerous cells, it is considered as tumor-associated antigen (TAA). Also, this group of antigens is envisaged in the present invention. The group of TAAs is typically subdivided into the group of shared TAAs and unique TAAs. Among the shared TAAs are antigens which are shared by several classes of cancers, whereas unique TAAs are believed to result from random somatic point mutations included by carcinogens, thus constituting neo-antigens unique expressed by individual tumors. The presence of such unique TAAs may advantageously be used for the preparation of specific antigen encoded by an mRNA according to the present invention.

The present invention preferably envisages the use of one or more of the following cancer antigens: MAGE-A1, NY-ESO-1, SSX-2, Gp-100, Melan-A/Mart-1, Tyrosinase, PSA, Mammaglobin-A, URLC10, GAA, OFA, cyclin 81/WT-1/CEF, VEGFR1, VEGFR2, TTK, MUC1-KLH, HER2, HPV16 E7, HPV16/18, CEA, KOC1, SL-701, WT1, p53, survivin, telomerase, GSK2302025A, MAGE-3.1, OVA BiP, CO16, DEPDC1, MPHOSPH1, ONT-10, GD2L and GD3L, TF, rsPSMA, MUC-2, PAP; KLH, STF-II, G17DT, ICT-107, LMP2A, NA17-A, NA17.A2, IMA901, hTERT, tyrosinase-related peptide 2 (TRP2), PANVAC, EBNA1/LMP2, TRICOM 5T4, MPHOSPH1 and DEPDC1. Furthermore, the present invention also relates to any combinations of the above-mentioned antigens, as well as derivatives or modified versions thereof or homologous protein/peptide sequences derived therefrom. Also envisaged is the employment of additional cancer antigens which may be discovered and described in the future. Also envisaged is the use of any other suitable antigen as known to the skilled person. Further details may be derived from Tagliamonte et al., 2014, Hum Vaccin Immunother, 10(11), 3332-3346. The term “cancer epitope” as used herein relates to a specific epitope of the MHC I or MHC II class present in a cancer antigen, e.g. as defined herein. The employment of cancer epitopes may, in specific embodiments, be combined with the additional characterization of a recipient's HLA allele background.

Particularly preferred is the employment of the cancer antigens NY-ESO-1, URLC10, G17DT, MART-1; NA17-A; gp100; hTERT, PAP, MPHOSPH1, DEPDC1, HPV16/18 or STF-II, or of epitopes present on these antigens.

The term “autoimmune disease antigen” as used herein relates to an antigenic substance composed of amino acids which leads to inappropriate immune responses that attack either self-tissues or innocuous environmental components. The autoimmune disease is thus typically, in most cases, a condition arising from abnormal immune responses to a normal body part, wherein almost all body parts may be involved. Autoimmune diseases involve the appearance of a reservoir of activated self-reactive lymphocytes and/or autoantibodies. The pathology may be restricted to certain organs or involve a particular tissue in different places. The use of autoimmune disease antigens in the context of the present invention involves the induction of immune tolerance against said antigens, e.g. as described herein. In specific embodiments of the present invention migratory immature Langerhans cells may be used for the induction of immune tolerance. In particular, a presentation as mentioned above may subsequently generate an immunological tolerance effect for said antigen if the DC is not activated. Accordingly, it is envisaged by the present invention, that a treatment of an autoimmune disease antigen comprises the delivery of an antigen leading to the subsequent presentation of it to T cells in the explicit absence of an immune stimulatory component. Such a presentation may subsequently generate an immunological tolerance effect for said antigen if the DC is not activated.

Autoimmune diseases for which antigens may be provided include, for example, Antiphospholipid-Syndrome (aPL syndrome), Pemphigus, Multiple Sclerosis (MS), Myasthenia gravis, Grave's disease, Goodpasture's syndrome, Microscopic angiitis, Granulomatosis with polyangiits, Systemic Autoimmune Rheumatic Diseases (SARD), Mixed Connective Tissue Disease, Systemic Lupus Erythematosus, Sjøgrens Syndrome, Systemic Sclerosis/CREST Syndrome, Polymyositis/Dermatomyositis, Autoimmune Thyroid Diseases, Idiopathic Myocarditis, Celiac Disease, Autoimmune Hepatitis, Primary Biliary Cirrhosis, ANCA Associated Diseases, Antiphospholipid Syndrome/Thromboembolic Syndrome, Anti-GBM Disease, Diabetes Mellitus, Pernicious Anemia, or Crohn's Disease. Suitable antigens would be known to the skilled person or can be derived form literature sources such as Wang et al., Nucleic Acids Research, 2017, 4S, D1, D769-D776. In particular embodiments, the present invention envisages the use of corresponding antigens such as beta2-GP1 for aPL syndrome, Dsg3 for Pemphigus, MBP, PLP and/or MOG-1 for Multiple Sclerosis, ACh receptor for Myasthenia gravis, TSH receptor for Grave's disease, Type IV collagen for Goodpasture's syndrome, p-ANCA for Microscopic angiitis, c-ANCA for Granulomatosis with polyangiits, DFS70 or lens epithelium-derived growth factor/transcription coactivator p75 (LEDGF/p75) for Systemic Autoimmune Rheumatic Diseases (SARD), U1-snRNP 68/70, U1-snRNP A, U1-snRNP C or U-snRNP B/B′ for Mixed Connective Tissue Disease, Sm, RNP/Sm, SmD, SmD1, SmD2, SmD or ribosomal phosophoprotein PO for Systemic Lupus Erythematosus, Ro/SS-A, or La/SS-B for Sjøgrens Syndrome, Centromere Protein B (CENP-B), Centromere Protein A (CENP-A), DNA Topoisomerase I (Sci-70) for Systemic Sclerosis/CREST Syndrome, Histidyl-tRNA synthetase (Jo-1), Threonyl-tRNA synthetase (PL-7), Alanyl-tRNA synthetase (PL-12), Glycyl-tRNA synthetase (EJ) or SRP54 for Polymyositis/Dermatomyositis, thyroid peroxidase (TPO; syn. MSA), thyroglobulin Autoimmune Thyroid Diseases, Tissue transglutaminase (tTG; syn. TGase-2 or Gliadin for Celiac Disease, Cytochrome p450 2D6, formiminotransferase cyclodeamidase (FTCD) for Autoimmune Hepatitis, M2, Branched chain 2-oxo acid dehydrogenase complex (BCOADC), OGDC-E2, or PDC-E2 Primary Biliary Cirrhosis, Myeloperoxidase (MPO) or Proteinase 3 (PR3) for ANCA Associated Diseases, beta2-glycoprotein 1 (beta2-GP1), formerly known as Apolipoprotein H (Apo H) for Antiphospholipid Syndrome/Thromboembolic Syndrome, glomerular basement membrane (GBM) for Anti-GBM Disease, glutamate decarboxylase (GAD65) for Diabetes Mellitus, intrinsic factor for Pernicious Anemia, or Glycoprotein 2 (GP2) for Crohn's Disease. Further envisaged are autoimmune disease epitopes present on an antigen, preferably as defined above. The term “autoimmune disease epitope” as used herein relates to a specific epitope of the MHC I or MHC 11 class present in an autoimmune disease antigen as defined herein above. The employment of autoimmune disease epitopes may, in specific embodiments, be combined with the additional characterization of a recipient's HLA allele background.

The term “bacterial antigen” as used herein relates to an antigenic substance composed of amino acids which is typically or in its original form produced or presented by a bacterium. They may be present in bacterial structures such as coats, capsules, cell walls, flagella, fimbriae or may be toxins of bacteria. Typically, such substances are displayed at the surface of bacteria. It is envisaged that certain polysaccharides and/or lipopolysaccharides which constitute major components on the surface of bacterial and may accordingly be seen as antigenic structures are mimicked by peptides or proteins, which accordingly provide the relevant antigen or epitope. The present invention envisages any suitable bacterial antigen composed of amino acids known to the skilled person. It is preferred that the bacterial antigen or the bacterial epitope is, comprises or is derived from tetanus toxoid, diphtheria toxoid. For example, the bacterial antigen or epitope is a T cell epitope derived from an antigen present in CRM, tetanus toxoid, diphtheria toxoid, Neisseria meningitidis outer membrane complex, or Hemophilus influenzae protein D. Further examples and details may be derived from suitable literature sources such as Detmer and Glenting, 2006, Microbial Cell Factories, 5, 23.

The term “viral antigen” as used herein relates to antigenic substances composed of amino acids which is typically or in its original form produced or presented by a virus. The viral antigen is typically a protein or peptide element, which is usually encoded by the virus genome. It may be presented on the surface of the virus, e.g. as a coat or envelop or part of it, or be an integral part of the virus core or of other viral structures, which may, for example be presented by cells after viral disintegration in the interior of a cell. Examples of viral antigens include viral structural elements such as a capsid protein, a matrix protein, an envelop protein etc., as well as non-structural proteins such as holins, movement proteins, NS proteins, e.g. NS2, NSP1 etc., or enzymatic activities encoded by viruses such as integrase, reverse transcriptase, neuraminidase, esterase etc. Preferred antigens are derived from hepatitis A virus (heat whole virus inactivated), hepatitis B, and human papilloma virus (HPV). Particularly preferred is the HepB-surface antigen. Also envisaged is the use of viral epitopes. The term “viral epitope” as used herein relates to a specific epitope of the MHC I or MHC II class present in a viral antigen as defined herein above. The employment of viral epitopes may, in specific embodiments, be combined with the additional characterization of a recipient's HLA allele background. Further information may be derived from suitable internet resources such as https://www.who.int/immunization/diseases/en/ (last visited on Dec. 4, 2018).

The term “parasitic antigen” as used herein, relates to antigenic substances composed of amino acids which is typically or in its original form produced or presented by parasites. The term “parasite” relates to parasites of mammals, preferably parasites of humans. Parasites typically belong to protozoa or metazoan. The major parasitic groups are parasitic protozoa and parasitic helminths. Protozoa are unicellular eukaryotes. Parasitic protozoa are typically divided into four groups based on their means of locomotion and mode of reproduction: flagellates, amebae, sporozoa, and ciliates. Within the group of flagellates there are intestinal and genitourinary flagellates such as Giardia and Trichomonas, as well as blood and tissue flagellates such as Trypanosoma and Leishmania. Examples of amebae include Entamoeba, Naegleria, and Acanthamoeba. The group of Sporozoa typically undergo a complex life cycle with alternating sexual and asexual reproductive phases and includes Cryptosporidium, Cyclospora, and Toxoplasma and the malarial parasites, i.e. Plasmodium species. This Sporozoa are typically intracellular parasites. Ciliates are complex protozoa bearing cilia. An example of this group is Balantidium coli, an intestinal ciliate of humans and pigs. Parasitic helminths usually belong to the groups of nematodes and plathelminthes. Examples of plathelminthes include trematodes, such as Fasciola hepatica, or cestoda such as Taenia. Also envisaged are Schistosoma or Filarial parasites such as Wuchereria bancrofti or Onchocerca volvulus. The present invention envisages antigens of any of the above-mentioned parasites or any other suitable parasite known to the skilled person. In specific embodiments, the antigen may be present in certain life cycle forms, e.g. on eggs. Particularly preferred is the employment of malaria antigens, e.g. protein structures displayed by Plasmodium during one of its life cycle forms such as cysteine-Rich Protective Antigen (CyRPA) which is a crucial component of a ternary complex, including Reticulocyte binding-like Homologous protein 5 (RHS) and the RH5-interacting protein (Ripr). The term “parasitic epitope” as used herein relates to a specific epitope of the MHC I or MHC II class present in a parasitic antigen as defined herein above. The employment of parasitic epitopes may, in specific embodiments, be combined with the additional characterization of a recipient's HLA allele background. Further information may be derived from suitable literature sources such as Tarleton, 2005, Cellular Microbiology, 7, 10, 1379-1386 or Higashi, 1988, Ann Rev Public Health, 9, 483-501.

The term “allergen” as used herein relates to non-self antigenic substances composed of amino acids capable of stimulating, for example, a type-I or type-IV hypersensitivity reaction in atopic individuals through Immunoglobulin E (IgE) responses or T cell-mediated response, respectively. Accordingly, the allergen is a type of antigen that produces an abnormally vigorous immune response in which the immune system defends the organism against a perceived threat that would otherwise be harmless to the body. Within the context of the present invention, the allergen typically comprises a protein or peptide encoded by an mRNA encapsulated in an LNP as described herein. Allergens can be found in a variety of sources, including dust mite excretion, pollen or pet dander. They can also be found in food such as peanuts, nuts, seafood or shellfish. A list of allergenic proteins, which is incorporated herein by reference, can be found at the SDAP (structural database of allergenic proteins), which can be found at http://fermi.utmb.edu. All allergens mentioned in said database are envisaged by the present invention. Also envisaged are any other allergen known to the skilled person. The term “epitope of an allergen” as used herein relates to a specific epitope of the MHC I or MHC II class present in an allergen as defined herein above. The employment of epitopes of an allergen may, in specific embodiments, be combined with the additional characterization of a recipient's HLA allele background. Further information would be known the skilled person or can be derived from suitable literature sources such as the “Opinion of the Scientific Panel on Dietetic Products, Nutrition and Allergies on a request from the Commission relating to the evaluation of allergenic foods for labelling purposes” as published in The EFSA Journal, 2004, 32, 1-197.

Optionally, e.g. in certain embodiments, the pharmaceutical composition as defined above comprises a pharmaceutically acceptable carrier or a pharmaceutical adjuvant. The term “pharmaceutically acceptable” means approved by a regulatory agency or other generally recognized pharmacopeia for use in animals, and more particularly in humans. The term “carrier” refers to a diluent, excipient, or pharmaceutical vehicle with which the LNP is administered. Such a carrier is pharmaceutically acceptable, i.e. is non-toxic to a recipient at the dosage and concentration employed. It is preferably isotonic, hypotonic or weakly hypertonic and has a relatively low ionic strength, such as provided by a sucrose solution. Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers. Suitable pharmaceutical excipients include starch, glucose, sucrose, gelatin, malt, rice, flour, chalk, silica gel, sodium stearate, glycerol monostearate, talc, sodium ion, dried skim milk, glycerol, propylene, glycol, water, ethanol and the like. The composition, if desired, can also contain minor amounts of wetting or emulsifying agents, or pH buffering agents. These compositions can take the form of, e.g., solutions, suspensions, emulsion, powders and the like. Examples of suitable pharmaceutical carriers are described, for example, in “Remington's Pharmaceutical Sciences” by E. W. Martin. Some other examples of substances which can serve as pharmaceutical carriers are sugars, such as glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; calcium carbonate; vegetable oils, such as peanut oils, cotton seed oil, sesame oil, olive oil, corn oil and oil of Theobroma; polyols such as propylene glycol, glycerine, sorbitol, manitol, and polyethylene glycol; agar; alginic acids; pyrogen-free water; isotonic saline; cranberry extracts and phosphate buffer solution; skim milk powder; as well as other non-toxic compatible substances used in pharmaceutical formulations such as Vitamin C, estrogen and Echinacea, for example. Wetting agents and lubricants such as sodium lauryl sulfate, as well as coloring agents, flavoring agents, lubricants, excipients, tabletting agents, stabilizers, anti-oxidants and preservatives, can also be present.

Generally, the ingredients may be supplied either separately or mixed together in unit dosage form, for example, as a dry lyophilised powder or water free concentrate in a hermetically sealed container such as an ampoule or sachette indicating the quantity of the LNP.

The term “pharmaceutical adjuvant” as used herein relates to additional ingredients such as chloroquine, protic polar compounds, such as propylene glycol, polyethylene glycol, glycerol, EtOH, 1-methyl L-2-pyrrolidone or their derivatives, or aprotic polar compounds such as dimethylsulfoxide (DMSO), diethylsulfoxide, di-n-propylsulfoxide, dimethylsulfone, sulfolane, dimethylformamide, dimethylacetamide, tetramethylurea, acetonitrile or their derivatives. The pharmaceutical adjuvant may further be one or more of a surfactant, wetting agent, dispersing agent, suspending agent, buffer, stabilizer or isotonic agent. The present invention also envisages any suitable pharmaceutical adjuvant as known to the skilled person. The above-mentioned compounds are added in conditions respecting pH limitations.

The pharmaceutical composition of the present invention can also comprise a preservative. Preservatives according to certain compositions of the invention include, without limitation: butylparaben; ethylparaben; imidazolidinyl urea; methylparaben; O-phenylphenol; propylparaben; quaternium-14; quaternium-15; sodium dehydroacetate; zinc pyrithione; and the like. The preservatives are used in amounts effective to prevent or retard microbial growth. Generally, the preservatives are used in amounts of about 0.1% to about 1% by weight of the total composition with about 0.1% to about 0.8% being preferred and about 0.1% to about 0.5% being most preferred.

The composition of the present invention can be administered to a subject or patient. The term “subject” or “patient” refers to a mammal. “Mammal” as used herein is intended to have the same meaning as commonly understood by one of ordinary skill in the art. Preferred mammals are primates, cows, sheep, goats, horses, dogs, cats, rabbits, rats, mice and the like. In particularly preferred embodiments, the subject is a human.

The term “administered” means administration of a therapeutically effective dose of the aforementioned pharmaceutical composition by any suitable route. By “therapeutically effective amount” is meant a dose that produces the effects for which it is administered in a patient. The exact dose will depend on the purpose of the treatment and will be ascertainable by one skilled in the art using known techniques. As is known in the art and described herein, adjustments for systemic versus localized delivery, age, body weight, general health, sex, diet, time of administration, drug interaction and the severity of the condition may be necessary, and will be ascertainable with routine experimentation by those skilled in the art.

It is preferred that the administration is localized, more preferably, that the administration is topical, in particular over or through the skin.

The pharmaceutical composition may be used in both human therapy and veterinary therapy, preferably in human therapy. The LNPs described herein may be administered in a physiologically acceptable carrier to a patient, as described herein. Depending upon the manner of administration, these elements may be formulated in a variety of ways as discussed herein.

The concentration of the compounds of a pharmaceutical composition according to the present invention may be further adjusted to the intended dosage regimen, the intended usage duration, the exact amount and ratio of all ingredients of the composition and further factors and parameter known to the person skilled in the art.

The LNPs as defined herein may be administered alone or in combination with other treatments. Combination treatments are envisioned for cancer immunotherapy, for example via co-administration of checkpoint inhibitors such as anti-CTLA-4 and anti-PD1 antibodies, for chemotherapy, for example by co-administration of alkylating agents or DNA and RNA polymerase inhibitors, for antiviral therapy, for example by co-administration of entry, protease or DNA and RNA polymerase inhibitors and for autoimmune disease therapy, for example by co-administration of glucocorticoids or immunophilin inhibitors.

The administration of the pharmaceutical composition can be done in a variety of ways. The administration may be, for example, topical, intradermal, transdermal, transfollicular, subcutaneous, intramuscular, intravenous, oral, sublingual, buccal, ophthalmic, otic, nasal, vaginal, or rectal or via inhalation.

Further, alternative routes of administration include, without limitation, ocular or intra-tumor administration or by intrasternal injection.

In further embodiments, the administration may be performed with a specific medical device, e.g. a needle, a vaccination gun, a plaster/adhesive, or an inhaler, as will be explained in detail below.

In certain preferred embodiments the administration is via hair follicles. The method is based on the finding that the pilosebaceous unit (consisting of the hair follicle and sebaceous gland) can play a role in the passive transport of pharmaceutical compostions into the skin. To reach the epidermis and egress from the skin into circulation, the pharmaceutical composition must additionally penetrate the keratinocyte layers surrounding the hair shaft.

Further envisaged administration routes include iontophoresis, microneedles, lasers and jet injectors.

Microneedles are typically considered as part of a transdermal patch, which is placed on the skin to deliver the pharmaceutical composition or a part of it to and across the skin. The microneedles are typically smaller than a human hair, composed of metals, Silicon or biodegradable polymers. Typically, the microneedles are provided in the form of an array. The use of the microneedles is advantageously virtually painless. A preferred embodiment of the microneedle approach is the nanopatch.

The term “nanopatch” used herein relates to an array of thousands of microprojections coated with pharmaceutical compositions that perforate into the outer layers of the skin when applied with an applicator device. The tips of Nanopatch's microprojections are typically coated with a vaccine material including the composition according to the present invention and release this material directly to the large numbers of immune cells immediately below the skin surface. The central element of this technology is the Nanopatch array itself which typically consists of a 1 cm square of silicon with ^{~}20,000 microprojections on its surface. The Nanopatch array penetrates through the protective outer skin layer (stratum corneum) and targets immune-activating material to the immune-cell rich layers just beneath the outermost skin layer utilising the microprojections with optimised spacing and length.

A further preferred route of administering is the topical route. Topical administration of the pharmaceutical composition of the present invention is useful when the desired treatment involves areas or organs readily accessible by topical administration. For a topical application, e.g. to the skin or mucosa, the pharmaceutical composition is preferably formulated as hydrogel patch, nanopatch, liquid, cream, ointment, paste, gel, lotion, tape, film, sublingual, buccal, tablet, spray, or suppository.

The term “hydrogel patch” as used herein relates to patches which are composed of a breathable non-woven cloth layered with an advanced adhering hydrogel which is typically protected by a transparent film cover. Hydrogel is a network of polymer chains that are water-insoluble, sometimes found as a colloidal gel in which water is the dispersion medium. Hydrogels are superabsorbent, natural or synthetic polymers an possess a degree of flexibility similar to natural tissue, due to their significant water content. According to certain embodiments, the hydropatch comprises the composition according to the present invention in a suitable amount.

A suitable paste comprises LNPs as described herein suspended in a carrier. Such carriers include, but are not limited to, petroleum, soft white paraffin, yellow petroleum jelly and glycerol.

The pharmaceutical composition may also be formulated with a suitable ointment comprising the active components suspended or dissolved in a carrier. Such carriers include, but are not limited to, one or more of glycerol, mineral oil, liquid oil, liquid petroleum, white petroleum, yellow petroleum jelly, propylene glycol, alcohols, triglycerides, fatty acid esters such as cetyl ester, polyoxyethylene polyoxypropylene compound, waxes such as white wax and yellow beeswax, fatty acid alcohols such as cetyl alcohol, stearyl alcohol and cetylstearylalcohol, fatty acids such as stearic acid, cetyl stearate, lanolin, magnesium hydroxide, kaolin and water.

Alternatively, the pharmaceutical composition may also be formulated with a suitable lotion or cream comprising the active components suspended or dissolved in a carrier. Such carriers include, but are not limited to, one or more of mineral oil such as paraffin, vegetable oils such as castor oil, castor seed oil and hydrogenated castor oil, sorbitan monostearat, polysorbat, fatty acid esters such as cetyl ester, wax, fatty acid alcohols such as cetyl alcohol, stearyl alcohol, 2-octyldodecanol, benzyl alcohol, alcohols, triglycerides and water.

The pharmaceutical composition may also be formulated as a tape or adhesive for transdermal application. The tape typically sticks to the skin as a patch and comprises the active components typically in a delayed release format. It is envisaged that the adhesive is a pressure sensitive adhesive wherein permeation enhancers, namely surfactants, fatty acids, terpenes and solvents, may have been introduced into the transdermal formulation. The pressure-sensitive adhesive typically begins as a highly viscous and sticky liquid and remains in the same form throughout their application life cycle. Alternatively, rubber-based pressure-sensitive adhesives may be employed which comprises of either natural or synthetic rubber, in addition to oils, resins and antioxidants as tackifier and stabiliser. Also envisaged are acrylic-based pressure-sensitive adhesive is prepared from acrylate esters, methacrylic acid, acrylamide, methacrylamide, N-alkoxyalkyl or N-alkyl-acrylamides without or with the addition of tackifier, or silicone-based pressure-sensitive adhesive is prepared mainly from gum and resin. The resin is a resultant product of the reaction of silicic or polysilicic hydrosol with trimethylchlorosilane.

A “sublingual administration” relates to pharmacological route of administration by which substances diffuse into the blood through tissues under the tongue. When the substance comes into contact with the mucous membrane beneath the tongue, it is absorbed. Because the connective tissue beneath the epithelium contains a profusion of capillaries, the substance then diffuses into them and enters the venous circulation. Typical administration forms for sublingual administration include sublingual tablets, sublingual strips, sublingual drops, sublingual spray, lozenges etc.

Similar to the sublingual administration, the buccal administration refers to a topical route of administration by which drugs held or applied in the buccal area (in the cheek) diffuse through the oral mucosa and enter either directly into the bloodstream or being taken up by Langerin-positive antigen presenting cells residing in the tissue. Buccal administration is believed to provide better bioavailability and a more rapid onset of action compared to oral administration because the medication does not pass through the digestive system and thereby avoids first pass metabolism. Modern approaches for buccal administration envisaged for the present invention include composite materials such as nanofiber-based mucoadhesive films. These materials typically consist of a mucoahesive layer, a reservoir layer to enable controlled release of the carrier. It is preferred that the materials comprise lipid-based nanoparticles and a protective backing layer. The further envisaged use of permeation enhancers such as surfactants, fatty acids as well as cationic and anionic amino acids may advantageously increase buccal bioavailability. Further details may be derived from suitable literature sources such as Morales et al., 2017, Curr Opin Pharmacol, 36, 22-28.

Alternatively, the pharmaceutical composition may also be formulated with a suitable gel comprising the active components suspended or dissolved in a carrier. Such carriers include, but are not limited to, one or more of water, glycerol, propyleneglycole, liquid paraffin, polyethylene, fatty oils, cellulose derivatives, bentonite and colloidal silicon dioxide.

The preparations according to the invention may generally comprise further auxiliaries as are customarily used in such preparations, e.g. preservatives, perfumes, antifoams, dyes, pigments, thickeners, surface-active substances, emulsifiers, emollients, finishing agents, fats, oils, waxes or other customary constituents, of a cosmetic or dermatological formulation, such as alcohols, polyols, polymers, foam stabilizers, solubility promoters, electrolytes, organic acids, organic solvents, or silicone derivatives.

The pharmaceutical composition according to the invention may comprise emollients. Emollients may be used in amounts, which are effective to prevent or relieve dryness. Useful emollients include, without limitation: hydrocarbon oils and waxes; silicone oils; triglyceride esters; acetoglyceride esters; ethoxylated glyceride; alkyl esters; alkenyl esters; fatty acids; fatty alcohols; fatty alcohol ethers; etheresters; lanolin and derivatives; polyhydric alcohols (polyols) and polyether derivatives; polyhydric alcohol (polyol) esters; wax esters; beeswax derivatives; vegetable waxes; phospholipids; sterols; and amides.

Thus, for example, typical emollients include mineral oil, especially mineral oils having a viscosity in the range of 50 to 500 SUS, lanolin oil, mink oil, coconut oil, cocoa butter, olive oil, almond oil, macadamia nut oil, aloa extract, jojoba oil, safflower oil, corn oil, liquid lanolin, cottonseed oil, peanut oil, purcellin oil, perhydrosqualene (squalene), caster oil, polybutene, odorless mineral spirits, sweet almond oil, avocado oil, calophyllum oil, ricin oil, vitamin E acetate, olive oil, mineral spirits, cetearyl alcohol (mixture of fatty alcohols consisting predominantly of cetyl and stearyl alcohols), linolenic alcohol, oleyl alcohol, octyl dodecanol, the oil of cereal germs such as the oil of wheat germ cetearyl octanoate (ester of cetearyl alcohol and 2-ethylhexanoic acid), cetyl palmitate, diisopropyl adipate, isopropyl palmitate, octyl palmitate, isopropyl myristate, butyl myristate, glyceryl stearate, hexadecyl stearate, isocetyl stearate, octyl stearate, octylhydroxy stearate, propylene glycol stearate, butyl stearate, decyl oleate, glyceryl oleate, acetyl glycerides, the octanoates and benzoates of (C12-C15) alcohols, the octanoates and decanoates of alcohols and polyalcohols such as those of glycol and glycerol, and ricin-oleates of alcohols and poly alcohols such as those of isopropyl adipate, hexyl laurate, octyl dodecanoate, dimethicone copolyol, dimethiconol, lanolin, lanolin alcohol, lanolin wax, hydrogenated lanolin, hydroxylated lanolin, acetylated lanolin, petrolatum, isopropyl lanolate, cetyl myristate, glyceryl myristate, myristyl myristate, myristyl lactate, cetyl alcohol, isostearyl alcohol stearyl alcohol, and isocetyl lanolate, and the like.

Moreover, the pharmaceutical composition according to the invention may also comprise emulsifiers. Emulsifiers (i.e., emulsifying agents) are preferably used in amounts effective to provide uniform blending of ingredients of the composition. Useful emulsifiers include (i) anionics such as fatty acid soaps, e.g., potassium stearate, sodium stearate, ammonium stearate, and triethanolamine stearate; polyol fatty acid monoesters containing fatty acid soaps, e.g., glycerol monostearate containing either potassium or sodium salt; sulfuric esters (sodium salts), e.g., sodium lauryl 5 sulfate, and sodium cetyl sulfate; and polyol fatty acid monoesters containing sulfuric esters, e.g., glyceryl monostearate containing sodium lauryl surfate; (ii) cationics chloride such as N(stearoyl colamino formylmethyl) pyridium; N-soya-N-ethyl morpholinium ethosulfate; alkyl dimethyl benzyl ammonium chloride; diisobutylphenoxytheoxyethyl dimethyl benzyl ammonium chloride; and cetyl pyridium chloride; and (iii) nonionics such as polyoxyethylene fatty alcohol ethers, e.g., monostearate; polyoxyethylene lauryl alcohol; polyoxypropylene fatty alcohol ethers, e.g., propoxylated oleyl alcohol; polyoxyethylene fatty acid esters, e.g., polyoxyethylene stearate; polyoxyethylene sorbitan fatty acid esters, e.g., polyoxyethylene sorbitan monostearate; sorbitan fatty acid esters, e.g., sorbitan; polyoxyethylene glycol fatty acid esters, e.g., polyoxyethylene glycol monostearate; and polyol fatty acid esters, e.g., glyceryl monostearate and propylene glycol monostearate; and ethoxylated lanolin derivatives, e.g., ethoxylated lanolins, ethoxylated lanolin alcohols and ethoxylated cholesterol.

The pharmaceutical composition according to the invention may also include a surfactant. Suitable surfactants may include, for example, those surfactants generally grouped as cleansing agents, emulsifying agents, foam boosters, hydrotropes, solubilizing agents, suspending agents and nonsurfactants (facilitates the dispersion of solids in liquids).

The surfactants are usually classified as amphoteric, anionic, cationic and nonionic surfactants. Amphoteric surfactants include acylamino acids and derivatives and N-alkylamino acids. Anionic surfactants include: acylamino acids and salts, such as, acylglutamates, acylpeptides, acylsarcosinates, and acyltaurates; carboxylic acids and salts, such as, alkanoic acids, ester carboxylic acids, and ether carboxylic acids; sulfonic acids and salts, such as, acyl isethionates, alkylaryl sulfonates, alkyl sulfonates, and sulfosuccinates; sulfuric acid esters, such as, alkyl ether sulfates and alkyl sulfates. Cationic surfactants include: alkylamines, alkyl imidazolines, ethoxylated amines, and quaternaries (such as, alkylbenzyldimethylammonium salts, alkyl betaines, heterocyclic ammonium salts, and tetra alkylammonium salts). And nonionic surfactants include: alcohols, such as primary alcohols containing 8 to 18 carbon atoms; alkanolamides such as alkanolamine derived amides and ethoxylated amides; amine oxides; esters such as ethoxylated carboxylic acids, ethoxylated glycerides, glycol esters and derivatives, monoglycerides, polyglyceryl esters, polyhydric alcohol esters and ethers, sorbitan/sorbitol esters, and triesters of phosphoric acid; and ethers such as ethoxylated alcohols, ethoxylated lanolin, ethoxylated polysiloxanes, and propoxylated polyoxyethylene ethers.

In case of the provision of the pharmaceutical composition as a film it may comprise a film former. Suitable film formers which are used in accord with the invention keep the composition smooth and even and include, without limitation: acrylamide/sodium acrylate copolymer; ammonium acrylates copolymer; Balsam Peru; cellulose gum; ethylene/maleic anhydride copolymer; hydroxyethylcellulose; hydroxypropylcellulose; polyacrylamide; polyethylene; polyvinyl alcohol; pvm/MA copolymer (polyvinyl methylether/maleic anhydride); PVP (polyvinylpyrrolidone); maleic anhydride copolymer such as PA-18 available from Gulf Science and Technology; PVP/hexadecene copolymer such as Ganex V-216 available from GAF Corporation; acryliclacrylate copolymer; and the like.

Generally, film formers can be used in amounts of about 0.1% to about 10% by weight of the total composition with about 1% to about 8% being preferred and about 0.1 DEG/O to about 5% being most preferred. Humectants can also be used in effective amounts, including: fructose; glucose; glulamic acid; glycerin; honey; maltitol; methyl gluceth-10; methyl gluceth-20; propylene glycol; sodium lactate; sucrose; and the like.

In a further embodiment of the present invention, the pharmaceutical composition may be administered via inhalation. The pharmaceutical preparations can accordingly be in the form of a spray, e.g. a pump spray or an aerosol. Typically, aerosols according to the present invention comprise the medicament or pharmaceutical composition, one or more chlorofluorocarbon propellants and either a surfactant or a solvent, such as ethanol. For instance, aerosol propellants like propellant 11 and/or propellant 114 and/or propellant 12 may be used. Further suitable propellants for aerosols according to the invention are propane, butane, pentane and others. Additional propellants which may be used and which are believed to have minimal ozone-depleting effects in comparison to conventional chlorofluorocarbons comprise fluorocarbons and hydrogen-containing chlorofluorocarbons. Additional aerosols for medicinal aerosol formulations are disclosed in, for example, EP 0372777. Typically, one or more adjuvants such as alcohols, alkanes, dimethyl ether, surfactants (including fluorinated and non-fluorinated surfactants, carboxylic acids, polyethoxylates etc) and conventional chlorofluorocarbon propellants in small amounts may be added to the formulations.

Further preferred is the use of 1,1,1,2-tetrafluoroethane in combination with both a cosolvent having greater polarity than 1,1,1,2-tetrafluoroethane (e.g. an alcohol or a lower alkane) and a surfactant in order to achieve a stable formulation of a pharmaceutical composition powder. Additionally, surfactants may be used as important components of aerosol formulations, in order to reduce the aggregation of the pharmaceutical composition and to lubricate, e.g. valves of a dispersing apparatus, if employed according to a further preferred embodiment of the present invention, thereby ensuring consistent reproducibility of valve actuation and accuracy of dose dispensed. Typically, the pharmaceutical composition according to the present invention may be pre-coated with surfactant prior to dispersal in 1,1,1,2-tetrafluoroethane.

In a further preferred embodiment of the present invention a pharmaceutical aerosol formulation may be dispersed with any suitable apparatus known to the person skilled in the art, preferably through a metered dose inhaler (MDI), a nebulizer, Rotahaler or an autohaler apparatus.

Oral delivery can be performed by complexing the composition as defined herein with a carrier capable of withstanding degradation by digestive enzymes in the gut of an animal. Examples of such carriers include plastic capsules or tablets, such as those known in the art.

A suitable suppository may comprise the composition as defined herein together with colloidal silicon dioxide, and an oleaginous base that includes triglycerides.

Assays, e.g. as derivable from known and qualified literature sources, may optionally be employed to help identify optimal ratios and/or dosage ranges for ingredients of pharmaceutical compositions of the present invention. The precise dose and the ratio between the ingredients of the pharmaceutical composition as defined herein above to be employed in the formulation will also depend on the route of administration, and the exact type of disease or disorder and should be decided according to the judgment of the practitioner and each patient's circumstances. Effective doses or ingredient ratios may be extrapolated from dose-response curves derived from in vitro or (animal) model test systems.

Typically, the attending physician and clinical factors may determine the dosage regimen. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 mg; however, doses below or above this exemplary range are also envisioned, especially considering the aforementioned factors.

In a preferred embodiment, the pharmaceutical composition as defined herein above is for use in the treatment or prevention of a disease or pathological condition.

A “disease” or “pathological condition” as used herein is any condition that would benefit from treatment with a pharmaceutical composition as defined above, in particular with an LNP as defined herein above.

The terms “treat” or “treatment”, unless otherwise indicated by context, refer to therapeutic treatment and/or prophylactic measures to prevent the outbreak or relapse of a disease or pathological condition, wherein the objective is to inhibit or slow down (lessen) an undesired physiological or pathological condition. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already having the condition or disorder as well as those prone to have the condition or disorder. The treatment may further, in specific embodiments, involve a single administration of a pharmaceutical composition as defined above, or multiple administrations. A corresponding administration scheme may be adjusted to the sex or weight of the patient, the disease, the pharmaceutical composition to be used, the general health status of the patient etc. For example, the administration scheme may contemplate an administration every 12 h, 24 h, 28 h, 72 h, 96 h, once a week, once very two weeks, once every 3 weeks, once a month etc. Also envisaged are pauses or breaks between administration phases. These regimens can of course be adjusted or changed by the medical practitioner in accordance with the patient's reaction to the treatment and/or the course of disease or of the pathological condition.

In a particularly preferred embodiment of the present invention, the pharmaceutical composition as defined herein above is for use in the treatment or prevention of cancer. The term “cancer” as used herein relates to a pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms typically show partial or complete lack of structural organization and functional coordination with the normal tissue and most invade surrounding tissues, metastasize to several sites, and are likely to recur after attempted removal and to cause the death of the patient unless adequately treated. The term thus also includes the existence and development of metastases. As used herein, the term “neoplasia” is used to describe all cancerous disease states and embraces or encompasses the pathological process associated with malignant hematogenous, ascitic and solid tumors. Representative cancers include, for example, stomach, colon, rectal, liver, pancreatic, lung, breast, cervix uteri, corpus uteri, ovary, prostate, including metastatic prostate cancer, testis, bladder, renal, brain/CNS, head and neck, throat, Hodgkin's disease, non-Hodgkin's lymphoma, multiple myeloma, leukemia, melanoma skin cancer, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma Also envisaged are further cancer forms known to the skilled person or derivable from suitable literature sources such as Pavlopoulou et al., 2015, Oncol Rep., 33, 1, 3-18.

The cancer may, in certain embodiments, be a refractory cancer. A cancer may be assumed to be residually present if a subject has undergone surgery as treatment for the cancer. Also envisaged are metastasizing cancer forms, e.g. of the above mentioned cancer forms.

The cancer forms mentioned above may preferably be treated by using tumor associated antigens or cancer antigens or cancer epitopes as described above, which are delivered to Langerhans cells as mRNA, which encodes for the respective antigens/epitopes, by targeted LNPs as described in this invention. These antigens or epitopes thereof are subsequently processed and presented by the Langerhans cells to further immune cells and activate these immune cells leading to an immune response, e.g. via CTLs or antibodies against entities, e.g. cells, showing said antigen or epitope. The activation of these immune cells, in turn, depends on the maturation and activation of Langerhans cells which may be achieved by co-administration of suitable adjuvants. Non-limiting examples of such adjuvants are TLR or Rig-I-like receptors (RLR) agonists.

It is also envisaged that different antigens and/or different epitopes, e.g. either derived from different cancer forms, or derived from different proteins or glycoproteins, which may be present on the same cancerous cell or in the same tumor, are provided. These antigens or epitopes may, for example, be provided in a multiantigen fusion protein or a multiepitope protein (e.g. comprising 2, 3, 4, 5, 6, 7, 8 or more epitopes), or as single antigen/epitope units which are packed together in a pharmaceutical composition. For example, mRNAs may encode for different antigens and may be mixed and subsequently be formulated in an LNP or group of LNPs as defined herein.

In a further particularly preferred embodiment of the present invention, the pharmaceutical composition as defined herein above is for use in the treatment or prevention of an autoimmune disease. The term “autoimmune disease” as used herein relates to inappropriate immune responses that attack either self-antigen, or for example, in specific diseases, non-self antigens. Examples of autoimmune diseases, which are envisaged by the present invention and can be treated with the described pharmaceutical composition, include Antiphospholipid-Syndrome (aPL syndrome), Pemphigus, Multiple Sclerosis (MS), Myasthenia gravis, Grave's disease, Goodpasture's syndrome, Microscopic angiitis, Granulomatosis with polyangiits, Systemic Autoimmune Rheumatic Diseases (SARD), Mixed Connective Tissue Disease, Systemic Lupus Erythematosus, Sjøgrens Syndrome, Systemic Sclerosis/CREST Syndrome, Polymyositis/Dermatomyositis, Autoimmune Thyroid Diseases, Idiopathic Myocarditis, Celiac Disease, Autoimmune Hepatitis, Primary Biliary Cirrhosis, ANCA Associated Diseases, Antiphospholipid Syndrome/Thromboembolic Syndrome, Anti-GBM Disease, Diabetes Mellitus, Pernicious Anemia, Vitiligo and Crohn's Disease. The treatment of autoimmune diseases as described above is based on the use of autoimmune disease antigens or autoimmune disease epitopes as described above, which are provided to Langerhans cells in the form of LNPs as described herein above. In particular, Langerhans cells are known to induce the expansion of regulatory T cells and the antigen-specific deletion of CTLs, i.e. an antigen-specific tolerance, in absence of co-stimulatory signals, i.e. without the co-delivery of adjuvants. In the context of this treatment scheme the autoimmune disease antigens or autoimmune disease epitopes as described above is hence preferably provided without co-delivery of any adjuvant. It is further preferred that any co-stimulatory signal which leads to an activation of Langerhans cells be inhibited by any suitable means known to the skilled person, e.g. immunosuppressants.

In yet another particularly preferred embodiment of the present invention, the pharmaceutical composition as defined herein above is for use in the treatment or prevention of a bacterial infection. The term “bacterial infection” relates to the infection of a patient, in particular of a human, with a pathogenic bacterium or a bacterium whose presence in the organism is unwanted, e.g. a part of a bacterial consortium or bioflora. Preferred examples include intracellular bacterial infections, e.g. conveyed by bacteria such as Chlamydophila, Ehrlichia, Rickettsia, Salmonella, Neisseria, Brucella, Mycobacterium, Nocardia, Listeria, Francisella, Legionella, or Yersinia. Further examples of pathogenic bacteria, whose infection is envisaged to be treated with the pharmaceutical composition according to the present invention include bacteria of the genus Bacillus, Bartonella, Bordetella, Borrelia, Campylobacter, Chlamydia, Clostridium, Corynebacterium, Enterococcus, Escherichia, Haemophilus, Helicobacter, Leptospira, Mycoplasma, Pseudomonas, Shigella, Staphylococcus, Streptococcus, Treponema, Ureaplasma and Vibrio. In specific embodiments, the bacterial infection to be treated may be an infection by one or more of the following species: Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica and Yersinia pseudotuberculosis.

In the context of a bacterial infection, the term “treating” includes any or all of: inhibiting the growth of bacteria, multiplication or replication of the pathogen that causes the infectious disease and ameliorating one or more symptoms of an infectious disease. The infections with bacteria as mentioned above may preferably be treated by using bacterial antigens or bacterial epitopes as described herein, or which are known to the skilled person, e.g. from suitable literature source such as Detmer and Glenting, 2006, Microbial Cell Factories, 5, 23, which are provided to Langerhans cells in the form of LNPs as described herein above. These antigens or epitopes thereof are subsequently processed and presented by the Langerhans cells to further immune cells and activate these immune cells leading to an immune response, e.g. mediated via T cells or antibodies, directed against entities, e.g. bacteria or parts thereof, from which the antigen or epitope originated. It is also envisaged that different antigens and/or different epitopes, e.g. either derived from different bacteria, or derived from different proteins or glycoproteins, which may be present on the same bacterium are provided. These antigens or epitopes may, for example, be provided in a multiantigen fusion protein or a multiepitope protein (e.g. comprising 2, 3, 4, 5, 6, 7, 8 or more epitopes), or as single antigen/epitope units which are packed together in a pharmaceutical composition. For example, mRNAs may encode for different molecular forms of the bacterial antigen/epitopes and may be mixed and subsequently be formulated in an LNP or group of LNPs as defined herein.

In another particularly preferred embodiment of the present invention, the pharmaceutical composition as defined herein above is for use in the treatment or prevention of a viral infection. The term “viral infection” as used herein refers to an infection and/or disease caused by a pathogenic virus or an infectious virus particle (virion). Examples of pathogenic viruses or virions, whose infection is envisaged to be treated with the pharmaceutical composition according to the present invention include viruses of the groups: Adenoviridae, Picornaviridae, Herpesviridae, Hepadnaviridae, Flaviviridae, Retroviridae, Orthomyxoviridae, Paramyxoviridae, Papillomaviridae, Rhabdoviridae, Togaviridae. In specific embodiments, the viral infection to be treated may be an infection by one or more of the following virus types: Adenovirus, Coxsackievirus, Epstein-Barr virus, Hepatitis A virus, Hepatitis B virus, Hepatitis C virus, Herpes simplex virus, type 1, Herpes simplex virus, type 2,Cytomegalovirus, Human herpesvirus, type 8, HIV, Influenza virus, Measles virus, Mumps virus, Human papillomavirus, Parainfluenza virus, Poliovirus, Rabies virus, Respiratory syncytial virus, Rubella virus, Varicella-zoster virus or SARS-CoV-2. Particularly preferred is the treatment of infections with Human papillomavirus, Influenza virus, SARS-CoV-2, Hepatitis A virus and Hepatitis B virus.

In the context of a viral infection, the term “treating” includes any or all of: inhibiting the growth of viruses, multiplication or replication of the pathogen that causes the infectious disease and ameliorating one or more symptoms of an infectious disease. The infections with viruses or virions as mentioned above may preferably be treated by using viral antigens or viral epitopes as described herein, or which are known to the skilled person, e.g. from suitable literature source such as Ansari et al., 2010, Nucleic Acids Res, 38, D847-D853 or from internet resources such as https://www.who.int/immunization/diseases/en/ (last visited on Dec. 4, 2018), which are provided to Langerhans cells in the form of LNPs as described herein above. The encoded antigens or epitopes thereof are subsequently processed and presented by the Langerhans cells to further immune cells and activate these immune cells leading to an immune response, e.g. via CTLs or antibodies against entities, e.g. viruses or parts thereof, from which the said antigen or epitope originated. It is also envisaged that different antigens and/or different epitopes, e.g. either derived from different viruses, or derived from different proteins or glycoproteins, which may be present on or in the same virus are provided. These antigens or epitopes may, for example, be provided in a multiantigen fusion protein or a multiepitope protein (e.g. comprising 2, 3, 4, 5, 6, 7, 8 or more epitopes), or as single antigen/epitope units which are packed together in a pharmaceutical composition. For example, mRNAs may encode for different molecular forms of the viral antigen/epitopes and may be mixed and subsequently be formulated in an LNP or group of LNPs as defined herein.

In yet another particularly preferred embodiment of the present invention, the pharmaceutical composition as defined herein above is for use in the treatment or prevention of a fungal infection. The term “fungal infection” relates to the infection a patient, in particular of a human, with a fungus. Such an infection may, but must not necessarily lead to a fungal disease, i.e. an infectious disease caused or transmitted by a fungus. Examples of fungal infections include aspergillosis, basidiobolomycosis, blastomycosis, candidosis, chromoblastomycosis, coccidioidomycosis, conidiobolomycosis, cryptococcosis, dermatophytosis, eumycetoma, histoplasmosis, lobomycosis, mucormycosis, non-dermatophyte superficial dermatomycoses, paracoccidioldomycosis, phaeohyphomycosis, pneumocystosis and scedosporiosis.

In the context of a fungal infection, the term “treating” includes any or all of: inhibiting the growth of fungi, multiplication or replication of the pathogen that causes the infectious disease and ameliorating one or more symptoms of the fungal infection. The fungal infections as mentioned above may preferably be treated by using fungal antigens or fungal epitopes which are provided to Langerhans cells in the form of LNPs as described herein above. These encoded antigens or epitopes thereof as defined herein are subsequently processed and presented by the Langerhans cells to further immune cells and activate these immune cells leading to an immune response, e.g. via T cells or antibodies against entities, from which the said antigen or epitope originated. It is also envisaged that different antigens and/or different epitopes, e.g. either derived from different fungi or derived from different proteins or glycoproteins, which may be present on or in the same fungus are provided. These antigens or epitopes may, for example, be provided in a multiantigen fusion protein or a multiepitope protein (e.g. comprising 2, 3, 4, 5, 6, 7, 8 or more epitopes), or as single antigen/epitope units which are packed together in a pharmaceutical composition. For example, mRNAs may encode for different molecular forms of the fungal antigen/epitopes and may be mixed and subsequently be formulated in an LNP or group of LNPs as defined herein.

In yet another particularly preferred embodiment of the present invention, the pharmaceutical composition as defined herein above is for use in the treatment or prevention of a graft-vs. host disease. The term “graft-vs. host disease” as used herein relates to a medical complication following the receipt of transplanted tissue from a genetically different person. Graft-vs. host disease is typically associated with stem cell transplants such as those that occur with bone marrow transplants. Graft-vs. host disease may also apply to other forms of transplanted tissues such as solid organ transplants. Without wishing to be bound by theory, it is assumed that the disease is caused by the fact that white blood cells of the donor's immune system which remain within the donated tissue (the graft) recognize the recipient (the host) as foreign (non-self). The white blood cells present within the transplanted tissue then typically attack the recipient's body's cells, leading to graft-vs. host disease. The graft-vs. host disease is different from a transplant rejection, which occurs when the immune system of the transplant recipient rejects the transplanted tissue; graft-vs. host disease, on the other hand, occurs when the donor's immune system's white blood cells attack the recipient's tissues. Graft-vs. host disease can also occur after a blood transfusion if the blood products used have not been irradiated or treated with an approved pathogen reduction system. The present invention envisages that the recipient of the donated tissue (the graft) is provided with an MHC antigen comprised in the donated tissue, e.g. an antigen of the graft or the donor of the tissue. This antigen may advantageously be provided to Langerhans cells via an LNP as defined herein. This step may typically be followed by an expansion and activation of MHC-specific regulatory T cells, which leads to an antigen-specific deletion of CTLs, i.e. an antigen-specific tolerance in the absence of co-stimulatory signals, e.g. without the co-delivery of adjuvants. In the context of this treatment scheme antigens or autoimmune disease epitopes are preferably provided without co-delivery of any adjuvant. It is further preferred that any co-stimulatory signal which leads to an activation of Langerhans cells be inhibited by any suitable means known to the skilled person, e.g. by immunosuppressants. Further details may be derived from suitable literature sources such as Sela et al., 2011, J. Exp. Med., 208, 12, 2489-2496.

In additional, particularly preferred embodiment of the present invention, the pharmaceutical composition as defined herein above is for use in the treatment or prevention of local or systemic inflammation. The term “inflammation” as used herein relates to complex biological response of body tissues to damaging or harmful stimuli, such as pathogens, damaged cells, or irritants. The inflammation is a protective response involving immune cells, blood vessels, and molecular mediators. The function of inflammation is to eliminate the initial cause of cell injury, clear out necrotic cells and tissues damaged from the original insult and the inflammatory process, and initiate tissue repair. Inflammation is a generic response, and therefore it is considered as a mechanism of Innate immunity. The process of inflammation is initiated by resident immune cells already present in the involved tissue, in particular resident macrophages, dendritic cells, histiocytes (Langerhans cells), Kupffer cells and mast cells. These cells possess surface receptors known as pattern recognition receptors (PRRs), which recognize two subclasses of molecules: pathogen-associated molecular patterns (PAMPs) and damage-associated molecular patterns (DAMPs). PAMPs are compounds that are associated with various pathogens, but which are distinguishable from host molecules. DAMPs are compounds that are associated with host-related injury and cell damage. At the onset of an infection, burn, or other injuries, these cells undergo activation (one of the PRRs recognize a PAMP or DAMP) and release inflammatory mediators responsible for the clinical signs of inflammation. Vasodilation and its resulting increased blood flow cause the redness and increased heat. Increased permeability of the blood vessels results in an exudation of plasma proteins and fluid into the tissue, which manifests itself as swelling. Some of the released mediators such as bradykinin increase the sensitivity to pain. The mediator molecules also alter the blood vessels to permit the migration of leukocytes, mainly neutrophils and macrophages, outside of the blood vessels into the tissue. The neutrophils migrate along a chemotactic gradient created by the local cells to reach the site of injury. The loss of function is probably the result of a neurological reflex in response to pain.

When inflammation overwhelms the host, i.e. becomes a “systemic inflammation”, a systemic inflammatory response syndrome is given. When it is due to infection, the syndrome is considered as sepsis. Vasodilation and organ dysfunction may occur with widespread infection that may lead to septic shock and death. In contrast thereto, a “local inflammation” is confined to the location or tissue region of the first harmful stimulus, injury or damage. Without wishing to be bound by theory, it is currently believed that Langerhans cells modulate regulatory T cells (Tregs), which in turn can shut down inflammatory responses, e.g. as described in Sharabi et al., 2018, Nature Reviews Drug Discovery, 17, 823-844. It is further assumed that Langerhans cells can induce an anti-inflammatory response via Tregs, as is derivable from suitable literature resources such as Stary et al., 2011, J Immunol, 186,1, 103-112. The envisaged treatment approach involves the provision of suitable compounds such as peptides or polypeptides encoded by mRNA encapsulated in an LNP of the present invention to Langerhans cells. There an anti-inflammatory response may accordingly be initiated. Further information can be derived from Bartneck et al., 2014, Nanomedicine, 10, 6, 1209-20.

In a further particularly preferred embodiment of the present invention, the pharmaceutical composition as defined herein above is for use in the treatment or prevention of allergy. The term “allergy” as used herein relates to a pathological condition caused by hypersensitivity of the immune system to a stimulus from the environment that normally, i.e. In a healthy subject cause little or no problem. The allergy is hence a condition in which the patient produces an abnormally vigorous immune response in which the immune system defends the organism against a perceived threat that would otherwise be harmless to the body. The underlying mechanism is assumed to involve immunoglobulin E antibodies (IgE), binding to an allergen, e.g. as defined herein above, and to a receptor on mast cells or basophils where it triggers the release of inflammatory chemicals such as histamine.

In a further particularly preferred embodiment of the present invention, the pharmaceutical composition as defined herein above is for use in hyposensitization. The term “hyposensitization” which is also known as desensitization or hypo-sensitization, is an allergen immunotherapy in the form of a medical treatment for some types of allergies which relies on the administration of increasing doses of allergens to drive immune responses from IgE production towards the induction of regulatory T cell responses, thereby promoting allergen-specific tolerance. The present invention can be used to directly induce regulatory T cell responses with higher efficiency via the selective delivery to Langerhans cells of allergens encoded by mRNA in LNPs, in the absence of adjuvants.

In yet another aspect the present invention relates to a method of treatment or prevention of cancer, of an autoimmune disease, of a bacterial infection, of a viral infection, of a fungal infection, or of a graft-vs. host disease, of a local or systemic inflammation, or of allergy, comprising administering to a subject a therapeutically effective amount of the pharmaceutical composition as defined above. The method may bed based on any suitable administration scheme as described herein above. The method of treatment, in particularly preferred embodiments, contemplates an administration via a topical, intradermal, transdermal, transfollicular, subcutaneous, intramuscular, intravenous, oral, sublingual, buccal, ophthalmic, otic, nasal, vaginal, or rectal administration or via inhalation.

In a further aspect the present invention relates to a method of hyposensitization. The present invention accordingly envisages that the allergic patient is provided with an antigenic allergen. This allergen may advantageously be provided to Langerhans cells as a pharmaceutical composition comprising LNPs as defined herein. It is particularly preferred that the antigenic allergen be provided in more than one dose. The number of doses and/or the concentration of allergen may be increased over the treatment period. This provision step, which may be repeated one or several times, is typically followed by an expansion and activation of antigenic allergen specific regulatory T cells, which lead to a deletion of allergen-specific T cells, i.e. an allergen-specific tolerance in the absence of co-stimulatory signals, e.g. without the co-delivery of adjuvants. In the context of this treatment scheme the allergens as described above are preferably provided without co-delivery of any adjuvant. It is further preferred that any co-stimulatory signal which leads to an activation of Langerhans cells be inhibited by any suitable means known to the skilled person, e.g. by immunosuppressants. Further details may be derived from suitable literature sources such as Sela et al., 2011, J. Exp. Med., 208, 12, 2489-2496.

The invention is further described in the following examples, which are not intended to limit the scope of the invention.

### EXAMPLES

### Example 1

### Lipid Conjugation

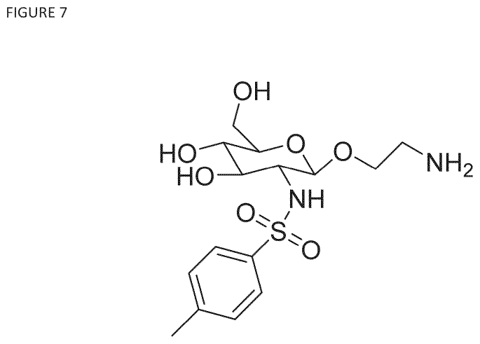

The glycomimetic Langerin ligand (depicted in FIG. 7) contains a terminal primary amine and was coupled to a NHS-bearing DSPE-containing lipid (‘DSPE-PEG2000’, #SUN DSPE-020GS, NOF Europe) via amide coupling: 2 mg ligand were dissolved in 900 μl 0.1 M sodium bicarbonate buffer (pH 8.4) and 0.125 equivalent lipids were dissolved in 100 μl DMF. The lipids were added dropwise in a pear shape flask and DMF was removed in vacuo (Heidolph). Unconjugated ligand was removed by dialysis with a Slide-A-Lyzer cassette (7MWCO, 0.5-3 ml, Thermo Fisher Scientific) against 0.5 L buffer over night with four times buffer exchanges under permanent stirring. Water was removed by freeze drying to obtain the ‘targeting lipid’ for subsequent lipid nanoparticle formulation.

Alexa Fluor 647 NHS Ester (#A37573, Thermo Fisher Scientific) was conjugated to the primary amine of NH₂—PEG2000-DSPE (#SUN DSPE-020PA, NOF Europe) via amide coupling. The lipid was dissolved in DMSO (1 mg/0.5 mL) and stirred in a pear-shaped flask. The dye (1.5 equiv.) was similarly dissolved in DMSO and added dropwise to the lipid. The reaction was stirred overnight in the dark at room temperature. DMSO was freeze dried (Alpha 2-4 LDplus, CHRIST) and the reaction product was dissolved in 0.1 M sodium bicarbonate buffer containing at pH8.4. Unconjugated dye was removed by dialysis with a Slide-A-Lyzer cassette (7MWCO, 0.5-3 ml, Thermo Fisher Scientific) against 0.5 L buffer over night with four times buffer exchanges under permanent stirring. Water was removed by freeze drying to obtain the ‘fluorescent dye lipid’ for subsequent lipid nanoparticle formulation.

### Example 2

### Preparation of Lipid Nanoparticles

t-/nt-LNP (1 to 4) were composed of different ionizable lipids [for t-/nt-LNP1: DLin-MC3-DMA (MedChemExpress, USA), for t-/nt-LNP 2: ALC-0315 (MedChemExpress, USA), for t-/nt-LNP 3: SM-102 (MedChemExpress, USA), and for t-/nt-LNP 4: DODMA (NOF Europe)], Cholesterol (Sigma-Aldrich, Austria), DSPC (NOF Europe), Structure (1-5)-PEG2000-DSPE (“targeting lipid” for t-LNP 1 to 4; see preparation in Example 1) or DSPE-PEG2000 (for nt-LNP 1 to 4; DSPE-020CN from NOF Europe) and optionally DSPE-PEG-Alexa 647 (for CleanCap® Enhanced Green Fluorescent Protein mRNA 5 moU; see preparation in Example 1). Generally, lipids were dissolved and mixed in ethanol 100% (Sigma Aldrich). The lipid mixture was then loaded into a syringe (Braun Injekt®, Germany). For the aqueous phase, sodium acetate buffer (50 mM, pH 4.5) was used. This RNase-free buffer (Invitrogen, Thermo Fisher Scientific) was used to dilute the CleanCap® Enhanced Green Fluorescent Protein mRNA 5moU (TriLink BioTechnologies, Inc., USA) or CleanCap® Ovalbumin mRNA 5moU (TriLink BioTechnologies, Inc., USA). An automatic syringe pump system (Pump 33 DDS (Dual Drive System) Syringe Pump, Harvard Apparatus) was loaded with the two syringes containing the dissolved lipids in one and the sodium acetate buffer containing the mRNA in the other. The pump was used to inject the two solutions via short tubes (TUBE ID 0.8 MM OD 1.6 MM WALLTHK 0.4 MM) attached to PEEK Capillary LUER lock adapters (Flangeless Fitting Delrin, Black ¼-28 Flat-Bottom, for 1/16″ OD, Luer Adapter ¼-28 F-to-F, IDEX-HS Corporation) to ensure pressure control into the inlets of the microfluidic chip (in-house developed chip design, manufactured by Wunderlichips GmbH, Switzerland) at a suitable speed. The final LNP solution was obtained from the chip outlet and collected in an Eppendorf flask. Subsequently, the LNP solution was dialyzed (Slide-A-Lyzer Dialysis cassette, 10 k MWCO, Thermo Scientific) overnight with ×1000 volume of buffer (PBS, pH 7.4). Formulated particles were stored at 4 °C before further experimental use.

### Example 3

### Generation of Epidermal Cell Suspensions

Epidermal cell suspensions were generated from the skin of transgenic human Langerin-DTR mice (huLang mice, see Bobr et al., 2010, J. Immunol., 185 (8), 4724-8). These huLang mice exclusively express the human Langerin receptor on and the diphtheria toxin receptor (DTR) in their Langerhans cells (LCs). After euthanasia, mice were epilated on back and belly and skin was harvested for immediate processing. After removal of subcutaneous fat with a scalpel, the skin was floated on a 0.6% Trypsin in PBS (Sigma Aldrich) solution and incubated for around 30 minutes at 37 °C. At the end of the incubation, the skin was floated on Fetal Bovine Serum (FBS) (Cytiva), before peeling off and placing the epidermis in RPMI medium supplemented with 10% FBS. The epidermis was then minced with scissors and further incubated for 20 minutes at 37 °C shaking prior to passing through a 70 μm strainer. After counting, the epidermal cells were plated in RPMI medium supplemented with 10% FBS and 1% Penicillin-Streptomycin (Sigma Aldrich).

### Example 4

### Stimulation of Epidermal Cell Suspensions and Detection of LNP Uptake and GFP mRNA Translation

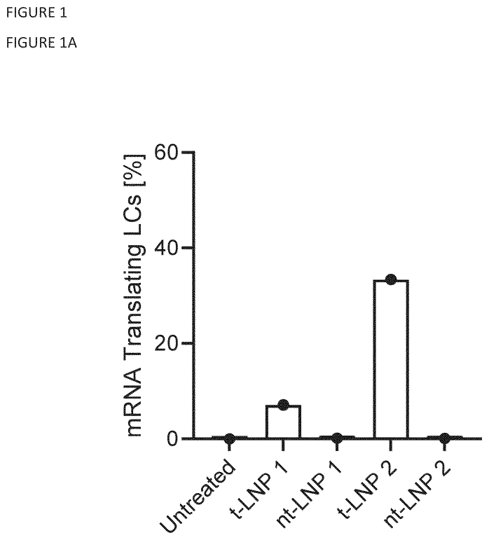

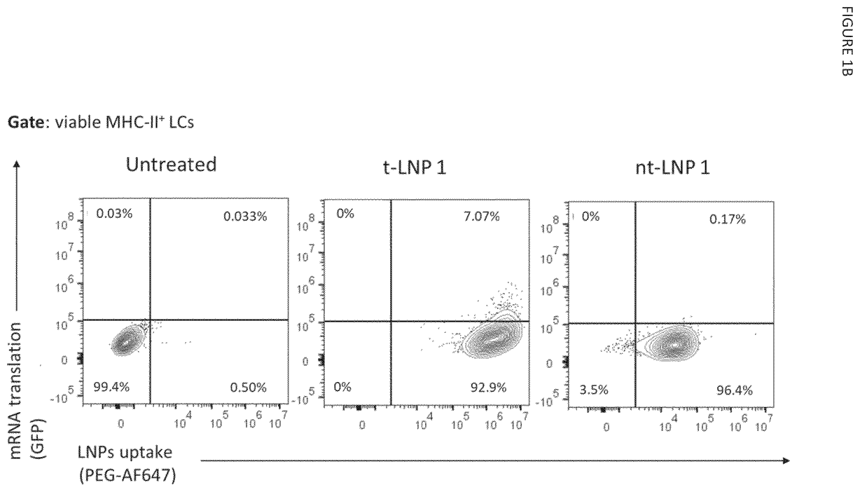

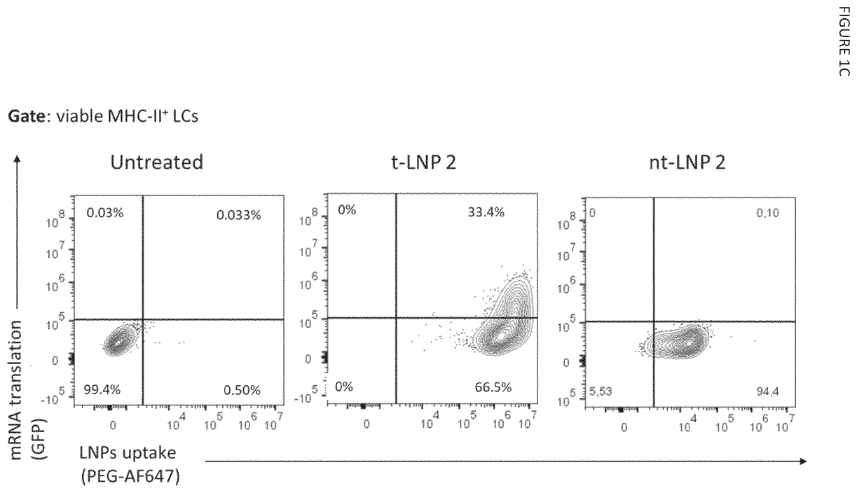

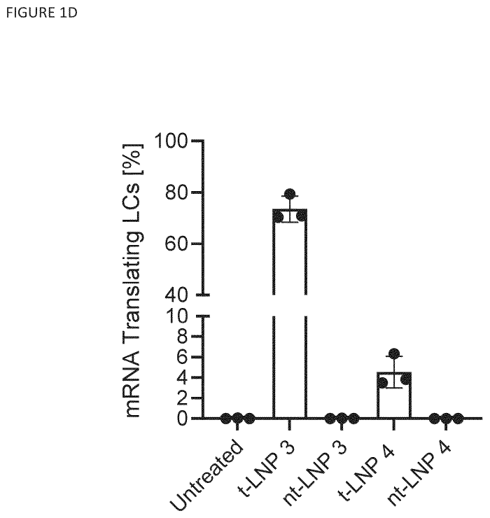

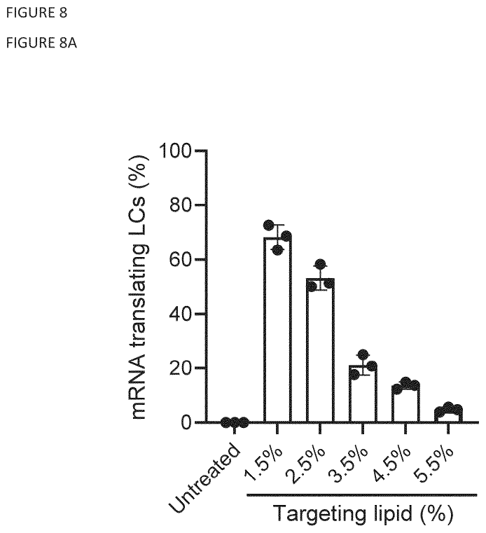

To analyse LNP uptake and GFP mRNA translation (as described in FIGS. 1 and 8), the epidermal cell suspension was stimulated for 16 hours. In FIG. 1 stimulation was performed with t-LNP 1, nt-LNP 1, t-LNP 2, nt-LNP 2, t-LNP 3, nt-LNP 3, t-LNP 4 or nt-LNP 4 at a final mRNA concentration of 0.25 μg/mL In FIG. 8 stimulation was performed with with t-LNP 3 containing different molar ratios of the targeting lipid at a final mRNA concentration of 0.25 μg/mL. The stimulant was then removed by washing with PBS and cells were prepared for flow cytometry staining. First, cells were stained with a viability dye (LIVE/DEAD™ Fixable Yellow Dead Cell Stain Kit, Thermo Fisher Scientific) according to the manufacturer's instruction. After washing, the cells were stained with Anti-mouse I-A/I-E (MHC-II) for 20 minutes at 4 °C. At the end of the incubation period, cells were washed and fixed using a fixing reagent (Cytofix, BD Biosciences) according to the manufacturer's instruction. Acquisition of samples was performed at a Beckman Coulter Cytoflex flow cytometer and analysis was performed with Flowjo v10.8. In the analysis, LCs were identified as viable, MHC-II⁺ cells. In the LCs, we assayed the GFP signal (indicating GFP-mRNA translation) and the AF647 signal (indicating cellular uptake of LNPs) across all the treatment conditions. mRNA translation and increased LNPs uptake was exclusively observed when using t-LNPs 1, t-LNPs 2, t-LNP 3 or t-LNP 4 but not the respective nt-LNP controls. These data indicate that the targeting ligand largely improves the capability to deliver LNPs to primary LCs and to induce mRNA translation in primary LCs.

### Example 5

### Isolation of OVA-Specific T Cells (OT-I or OT-II T Cells) and In Vitro Co-Cultures with Epidermal Cell Suspensions

Naïve CD8+(OT-I T cells) or CD4+(OT-II T cells) OVA-specific T cells were isolated from OT-I or OT-II mice, respectively, using naïve T cell isolation kits from Miltenyi according to manufacturer's instructions. Isolated OT-I or OT-II T cells were labelled with 2.5 μM carboxyfluorescein succinimidyl ester (CFSE) using the CFSE Cell Division Tracker Kit (Biolegend) according to manufacturer's instructions.

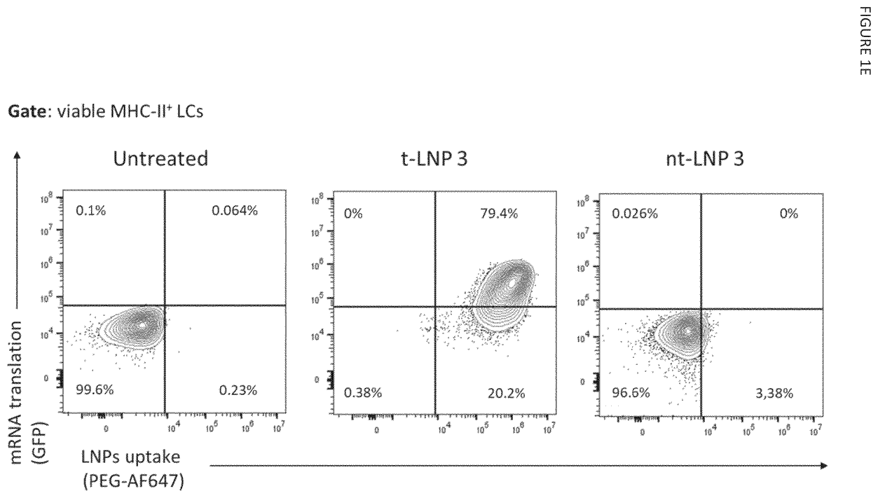

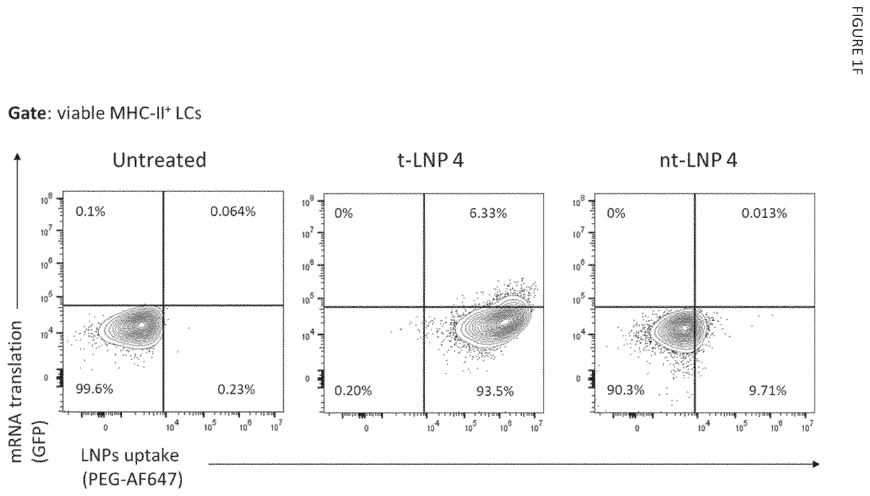

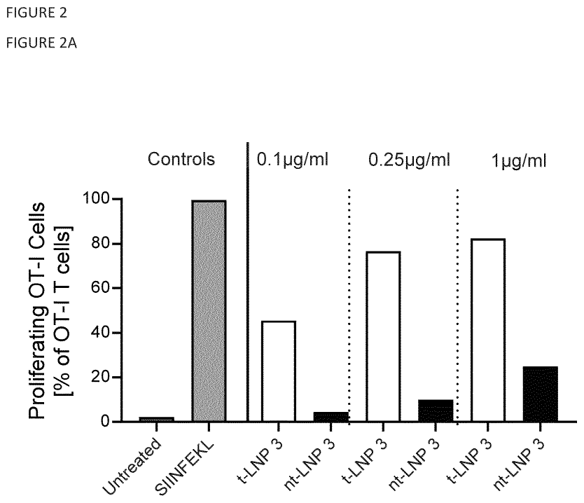

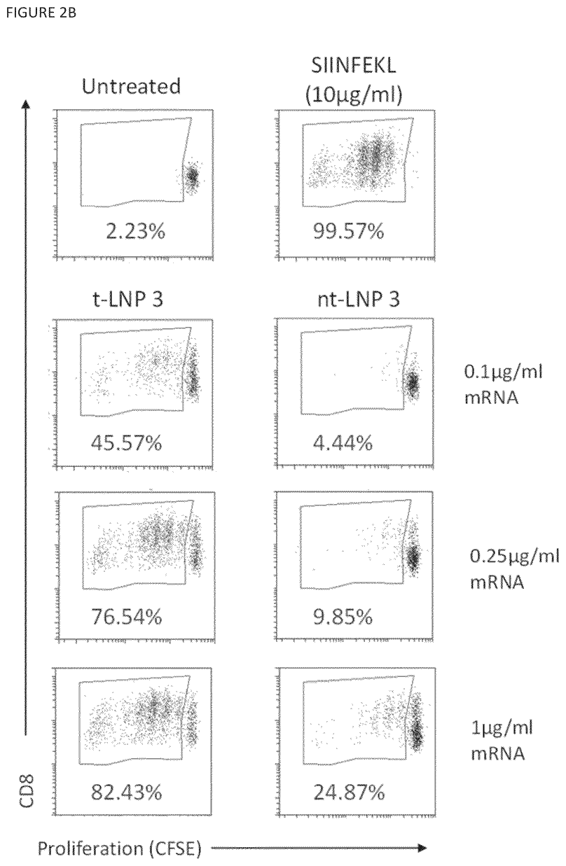

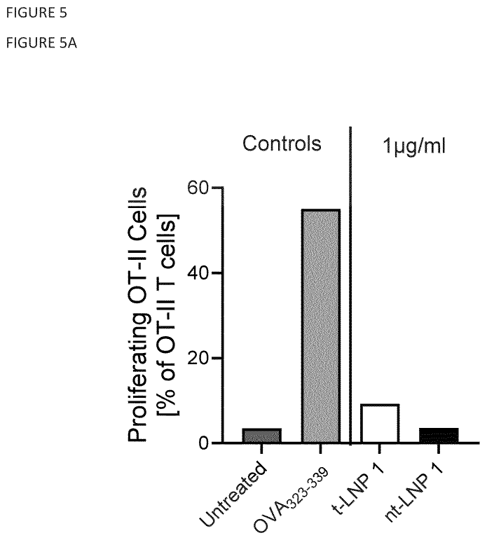

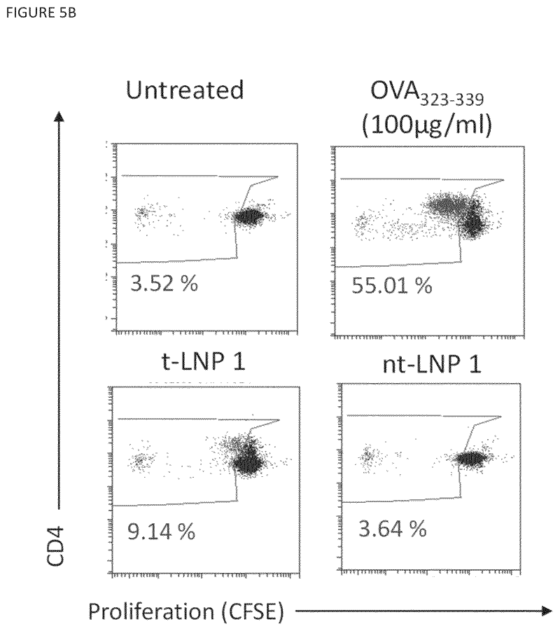

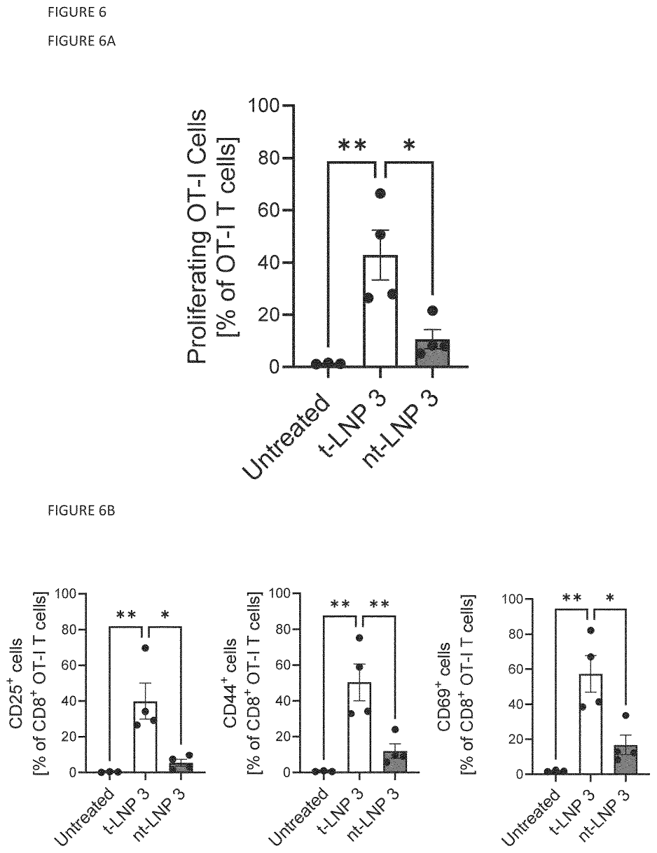

For the experiments described in FIG. 2-6, isolated CFSE labelled OT-I or OT-II T cells were co-cultured with EC suspension, prepared from untreated (see FIGS. 2-5) huLang mice or from huLang mice upon intradermal injection of LNPs (FIG. 6; described in Example 6), at a ratio of 20:1 (EC:OT-I/II) at 37 °C for 72 h. Following stimulants were added to co-cultures in vitro for experiments described in FIG. 2-5: nt-/t-LNP 1 containing mRNA encoding for OVA (further specified in Table 2 depicted in FIG. 10) at a final mRNA concentration of 1 μg/ml; nt-/t-LNP 3 formulations containing mRNA encoding for OVA (further specified in Table 2 depicted in FIG. 10) at final mRNA concentrations of 0.1 μg/ml, 0.25 μg/ml and 1 μg/ml; SIINFEKL peptide (final concentration of 100 μg/ml or 10 μg/ml); OVA₃₂₃₋₃₃₉ peptide (final concentration of 100 μg/ml or 10 μg/ml).

### Example 6

### Intradermal Injection of Mice with LNP Formulations

For the experiment described in FIG. 6, huLang mice were anesthetized and t-LNP 3 or nt-LNP 3 formulations containing 0.25 μg of OVA mRNA were injected intradermally into the ears (0.125 μg of mRNA-LNPs in 25 μl of PBS per ear) with a conventional hypodermic needle (30G). EC suspensions of the treated ears and untreated back (as negative controls) were prepared from the mice three hours after injection and used for co-culture with CFSE labelled OT-I T cells, which were prepared as described in Example 5.

### Example 7

### Flow Cytometric Analysis of OT-I/II T Cell Activation and Proliferation

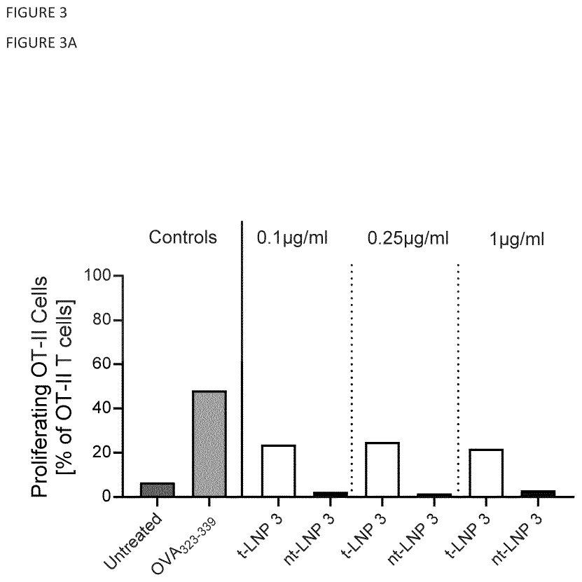

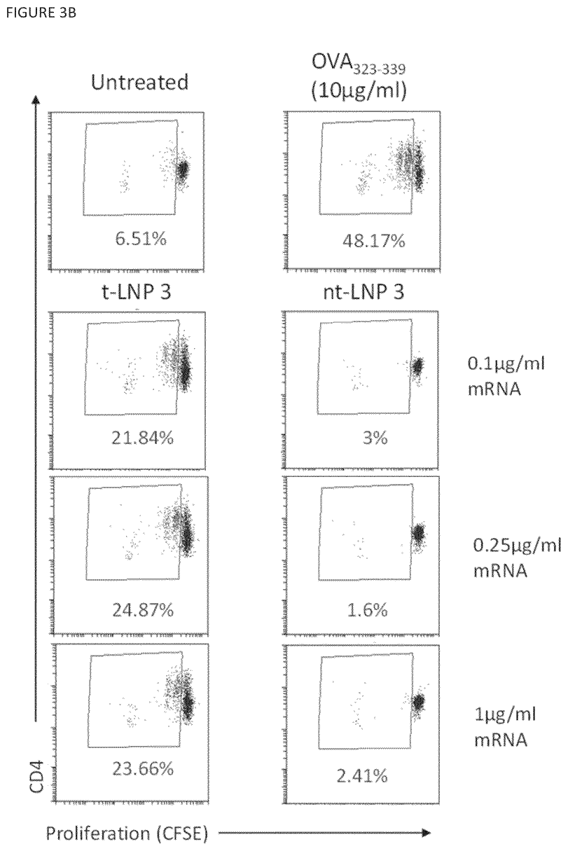

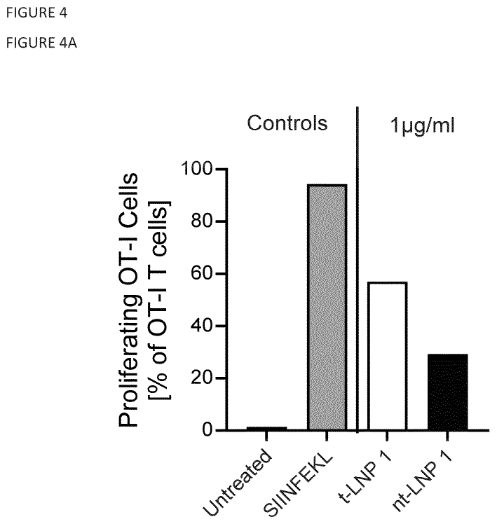

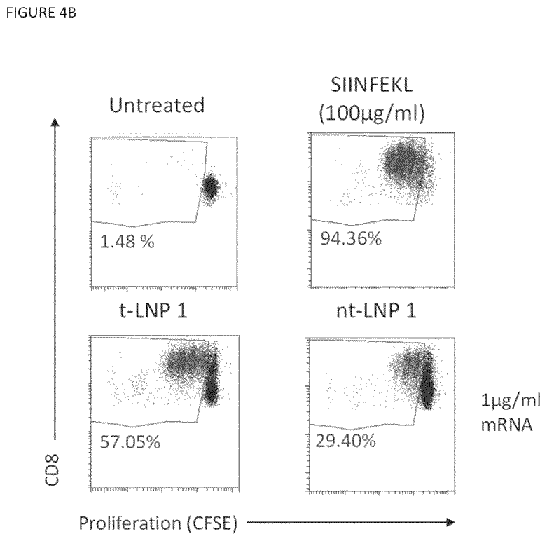

72 h after co-culture, OT-I or OT-II T cell activation and/or proliferation was analyzed by flow cytometric staining. First, cells were stained with a viability dye (LIVE/DEAD™ Fixable Yellow Dead Cell Stain Kit or LIVE/DEAD™ Fixable Near-IR Dead Cell Stain Kit, Thermo Fisher Scientific) according to the manufacturer's instruction. After washing, the cells were stained with following antibodies for 20 minutes at 4 °C: anti-mouse CD45.1, anti-mouse TCR Vβ5.1/5.2, anti-mouse CD8α (FIG. 2); anti-mouse CD45.1, anti-mouse CD3, anti-mouse CD4 (FIG. 3); anti-mouse CD4, anti-mouse CD8α, anti-mouse CD90.2 (FIGS. 4 and 5); anti-mouse CD45.1, anti-mouse TCR Vβ5.1/5.2, anti-mouse CD8α, anti-mouse CD44, anti-mouse CD25, anti-mouse CD69 (FIG. 6). At the end of the incubation period, cells were washed and fixed using a fixing reagent (Cytofix, BD) according to the manufacturer's instruction. Acquisition of samples was performed at a Beckman Coulter Cytoflex flow cytometer and analysis was performed with CytExpert 2.4.0.28 software.

1. A pharmaceutical composition comprising at least one lipid nanoparticle (LNP) specific for targeting Langerhans cells (LC), wherein the LNP encapsulates at least one mRNA, is capable of specifically binding to the receptor Langerin and facilitates Langerin-mediated uptake and intracellular delivery of said mRNA and its translation into at least one protein or peptide.

2. The pharmaceutical composition of claim 1, wherein the LNP comprises a targeting lipid, which is capable of specifically binding to the receptor Langerin, of the general formula (I) 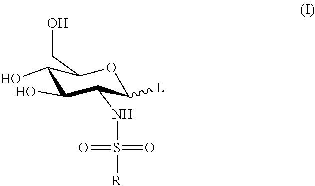
wherein L is a lipid, a modified lipid, such as a phospholipid, 1,2-distearoyl-sn-glycero-3-phosphoethanolamine (DSPE), oxyglutaryl aminopropyl polyethyleneglycol-carbamyl distearoylphosphatidyl-ethanolamine (DSPE-PEG), a membrane lipid, or a modified phosphatidylcholine; and
wherein R is a phenyl, a mono-, di- or trisubstituted phenyl,
wherein substituents of the phenyl are independently selected from the group consisting of —NH₂, —OH, —OCH₃, —C(O)CH₃, C(O)NH₂, —C(O)NHCH₃, —CH₂OH —NHC(O)CH₃, —F, —Cl, —Br, —NO₂, —CN, C₁-C₄ alkyl, naphtyl and phenyl.

3. The pharmaceutical composition of claim 2, wherein the LNP additionally comprises at least one stealth lipid, preferably DSPE-PEG (N-(Methylpolyoxyethylene oxycarbonyl)-1,2-distearoyl-sn-glycero-3-phosphoethanolamine), DMG-PEG (1,2-dimyristoyl-rac-glycero-3-methoxypolyethylene glycol) or DSG-PEG (1,2-Distearoyl-rac-glycero-3-methylpolyoxyethylene), more preferably DSPE-PEG2000 N-[Carbonyl(methoxypolyethylene glycol)]-1,2-distearoyl-sn-glycero-3-phosphoethanolamine.

4. The pharmaceutical composition of claim 2 or 3, wherein the LNP additionally comprises at least one helper lipid, preferably DSPC (1,2-Distearoyl-sn-glycero-3-phosphocholine), DSPE (1,2-Distearoyl-sn-glycero-3-phosphoethanolamine) or DOPE (1,2-Dioleoyl-sn-glycero-3-phosphoethanolamine).

5. The pharmaceutical composition of any one of claims 2 to 4, wherein the LNP additionally comprises at least one structural lipid, preferably cholesterol or a cholesterol analog or a combination thereof.

6. The pharmaceutical composition of any one of claims 2 to 5, wherein the LNP additionally comprises at least one ionizable lipid, preferably DLin-MC3-DMA ((6Z,9Z,28Z,31Z)-Heptatriaconta-6,9,28,31-tetraen-19-yl 4-(dimethylamino) butanoate), SM-102 (8-[(2-hydroxyethyl)[6-oxo-6-(undecyloxy)hexyl]amino]-octanoic acid, 1-octylnonyl ester), ALC-0315 (2-hexyl-decanoic acid, 1,1′-[[(4-hydroxybutyl) imino]di-6,1-hexanedlyl]ester), or DODMA (1,2-Dioleyloxy-3-dimethylamino-propane).

7. The pharmaceutical composition of any one of claims 2 to 6, wherein the mRNA is unmodified; or modified to optimize stability, translation efficiency or immunogenicity.

8. The pharmaceutical composition of any one of claims 2 to 7, wherein the targeting lipid is of one of the following formulae:

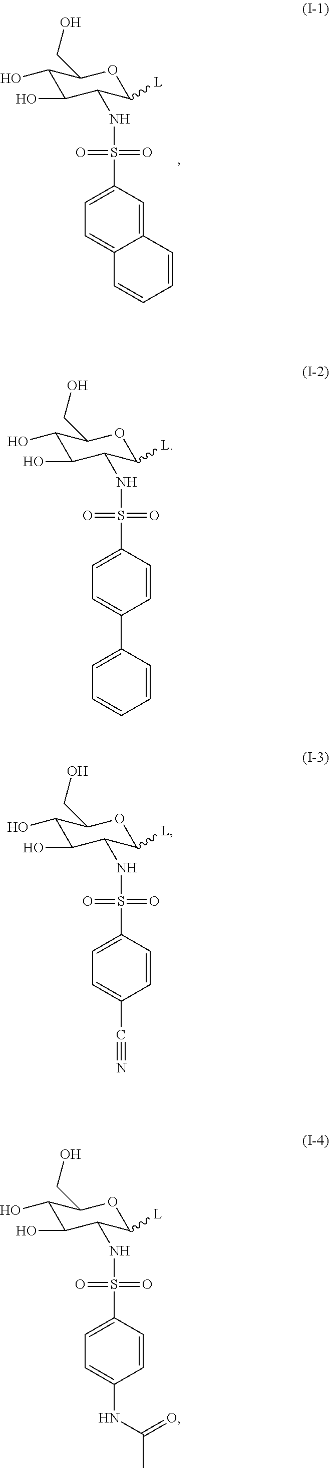

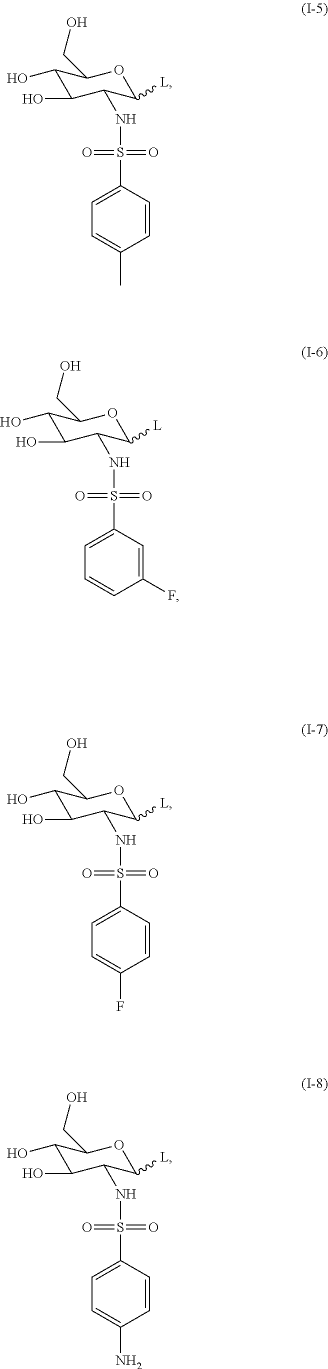

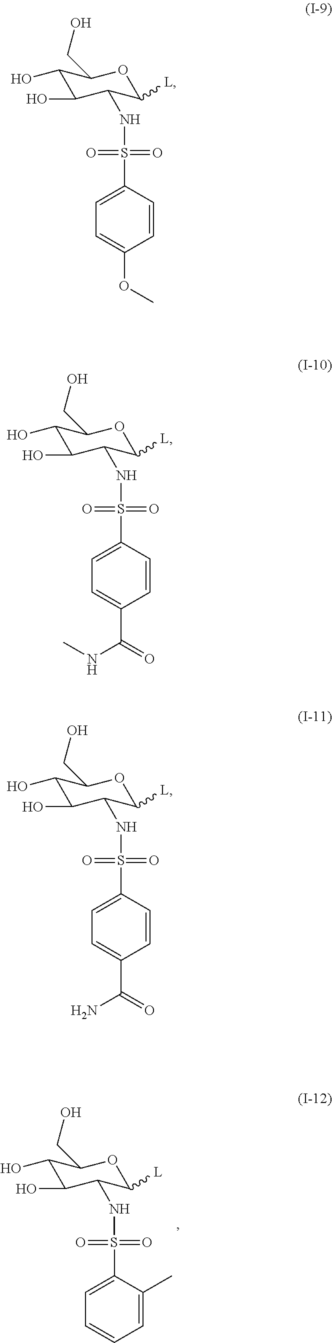

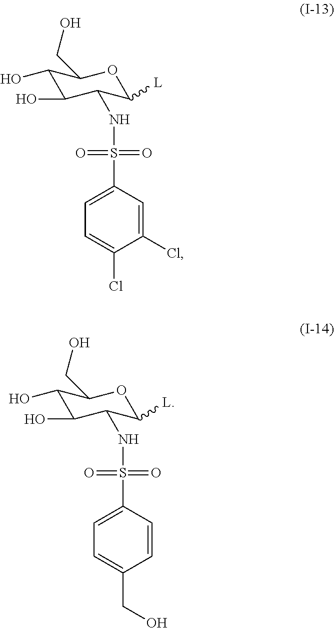

9. The pharmaceutical composition of any one of claims 2 to 8, wherein L is of the following formula (II): 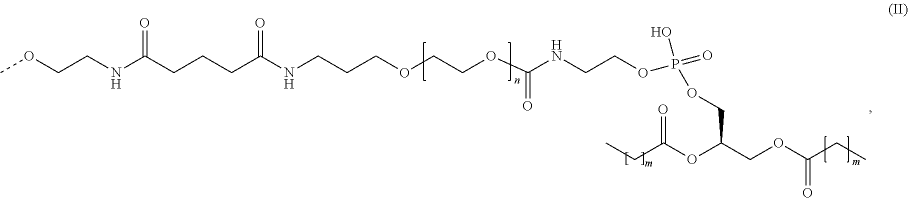
wherein n is an integer from 0 to 150 and m is an integer from 1 to 30.

10. The pharmaceutical composition of claim 9, wherein the targeting lipid is of the following formula (III) 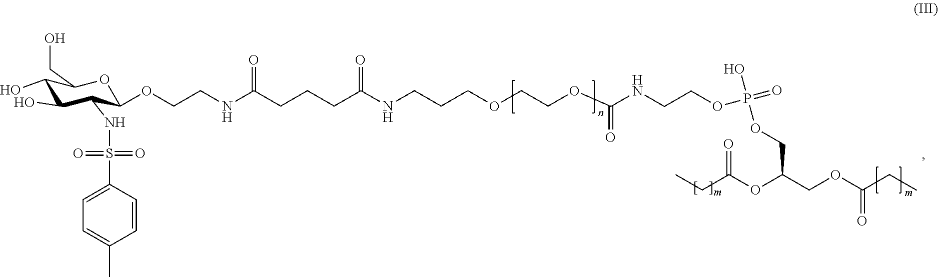
wherein n is an integer from 0 to 150 and m is an integer from 1 to 30.

11. The pharmaceutical composition of any one of claims 1 to 10, wherein the LNP has a size of about 30 to 250 nm.

12. The pharmaceutical composition of any one of claims 1 to 11, wherein the pharmaceutical composition comprises a solvent or a combination of a solvent with a further compound, or is provided as a dry composition, preferably as powder composition.

13. The pharmaceutical composition of claim 12, wherein said solvent is H₂O, aqueous sucrose solution, phosphate buffered saline, aqueous sodium chloride solution, tricine buffer, or HEPES buffer.

14. The pharmaceutical composition of claim 12 or 13, wherein said further compound is DMSO, propylene glycol, or oleic acid, preferably DMSO in a concentration of up to 10%.

15. The pharmaceutical composition of any one of claims 2 to 14, wherein the amount of the targeting lipids in the LNP is about 0.01 to 5 mol %, preferably about 0.1 to 3 mol %, more preferably about 0.25 to 2 mol %.

16. The pharmaceutical composition of any one of claims 3 to 15, wherein the amount of the stealth lipids in the LNP is about 0 to 5 mol %, preferably about 0.1 to 2 mol %.

17. The pharmaceutical composition of any one of claims 4 to 16, wherein the amount of the helper lipids in the LNP is about 5 to 15 mol %, preferably about 10 mol %.

18. The pharmaceutical composition of any one of claims 5 to 17, wherein the amount of the structural lipids in the LNP is about 20 to 60 mol %, preferably 35 to 40 mol %.

19. The pharmaceutical composition of any one of claims 6 to 18, wherein the amount of the ionizable lipids or of the combination of ionizable lipids in the LNP is about 10 to 70 mol %, preferably about 15 to 60 mol %, more preferably about 20 to 55 mol %.

20. The pharmaceutical composition of any one of claims 1 to 19, wherein said mRNA encodes for a pharmaceutically or immunologically active compound.

21. The pharmaceutical composition of any one of claims 1 to 20, wherein said mRNA encodes for any one of:
(i) a cancer antigen or epitope;
(ii) an autoimmune disease antigen or epitope;
(iii) a bacterial antigen or epitope;
(iv) a viral antigen or epitope;
(v) a parasitic antigen or epitope; or
(vi) an allergen, or an epitope of an allergen.

22. The pharmaceutical composition of any one of claims 1 to 21, wherein said pharmaceutical composition is suitable for topical, intradermal, transdermal, transfollicular, subcutaneous, intramuscular, intravenous, oral, sublingual, buccal, ophthalmic, otic, nasal, vaginal, or rectal administration or via inhalation.

23. The pharmaceutical composition of any one of claims 1 to 22, wherein said pharmaceutical composition is provided as a patch, nanopatch, cream, ointment, paste, gel, powder, lotion, tape, film, tablet, spray, suppository, or in the form of a solution for injection.

24. The pharmaceutical composition of any one of claims 1 to 23 is to be administered without or with a medical device, such as a needle, a microneedle device, a vaccination gun, a plaster, or an inhaler.

25. The pharmaceutical composition of any one of claims 1 to 24, for use in the treatment or prevention of cancer, of an autoimmune disease, of a bacterial infection, of a viral infection, of a fungal infection or of a graft-vs. host disease, of a local or systemic inflammation, of an allergy, or for hyposensitization.

26. A method of treatment or prevention of cancer, of an autoimmune disease, of a bacterial infection, of a viral infection, of a fungal infection, of a graft-vs. host disease, of a local or systemic Inflammation, of an allergy, or for hyposensitization comprising administering to a subject a therapeutically effective amount of the pharmaceutical composition as defined in any one of 1 to 24.

27. The method of claim 26, wherein said administration is a topical, intradermal, transdermal, transfollicular, subcutaneous, intramuscular, intravenous, oral, sublingual, buccal, ophthalmic, otic, nasal, vaginal, or rectal administration or via inhalation.


SEQUENCE LISTING (13 sequences)

SEQ ID NO: 1 - protein, 328 aa, Homo sapiens
MTVEKEAPDAHFTVDKQNISLWPREPPPKSGPSLVPGKTPTVRAALICLTLVLVASVLLQAVLYPRFMGTISDVKTNVQLLKGRVDNISTLDSEIKKNSDGMEAAGVQIQMVNESLGYVRSQFLKLKTSVEKANAQIQILTRSWEEVSTLNAQIPELKSDLEKASALNTKIRALQGSLENMSKLLKRQNDILQVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEP

SEQ ID NO: 2 - DNA, 987 bp, Homo sapiens
  source 1..987: mol_type = other DNA
atgactgtggagaaggaggcccctgatgcgcacttcactgtggacaaacagaacatctccctctggccccgagagcctcctcccaagtccggtccatctctggtcccggggaaaacacccacagtccgtgctgcattaatctgcctgacgctggtcctggtcgcctccgtcctgctgcaggccgtcctttatccccggtttatgggcaccatatcagatgtaaagaccaatgtccagttgctgaaaggtcgtgtggacaacatcagcaccctggattctgaaattaaaaagaatagtgacggcatggaggcagctggcgttcagatccagatggtgaatgagagcctgggttatgtgcgttctcagttcctgaagttaaaaaccagtgtggagaaggccaacgcacagatccagatcttaacaagaagttgggaagaagtcagtaccttaaatgcccaaatcccagagttaaaaagtgatttggagaaagccagtgctttaaatacaaagatccgggcactccagggcagcttggagaatatgagcaagttgctcaaacgacaaaatgatattctacaggtggtttctcaaggctggaagtacttcaaggggaacttctattacttttctctcattccaaagacctggtatagtgccgagcagttctgtgtgtccaggaattcacacctgacctcggtgacctcagagagtgagcaggagtttctgtataaaacagcggggggactcatctactggattggcctgactaaagcagggatggaaggggactggtcctgggtggatgacacgccattcaacaaggtccaaagtgtgaggttctggattccaggtgagcccaacaatgctgggaacaatgaacactgtggcaatataaaggctccctcacttcaggcctggaatgatgccccatgtgacaaaacgtttcttttcatttgtaagcgaccctatgtcccatcagaaccgtga

SEQ ID NO: 3 - protein, 328 aa, Homo sapiens
MTVEKEAPDAHFTVDKQNISLWPREPPPKSGPSLVPGKTPTVRAALICLTLVLVASVLLQAVLYPRFMGTISDVKTNVQLLKGRVDNISTLDSEIKKNSDGMEAAGVQIQMVNESLGYVRSQFLKLKTSVEKANAQIQILTRSWEEVSTLNAQIPELKSDLEKASALNTKIRALQGSLENMSKLLKRQNDILQVVSQGWKYFKGNFYYFSLIPKTWYSAEQFCVSRNSHLTSVTSESEQEFLYKTAGGLIYWIGLTKAGMEGDWSWVDDTPFNKVQSARFWIPGEPNNAGNNEHCGNIKAPSLQAWNDAPCDKTFLFICKRPYVPSEP

SEQ ID NO: 4 - DNA, 987 bp, Homo sapiens
  source 1..987: mol_type = other DNA
atgactgtggagaaggaggcccctgatgcgcacttcactgtggacaaacagaacatctccctctggccccgagagcctcctcccaagtccggtccatctctggtcccggggaaaacacccacagtccgtgctgcattaatctgcctgacgctggtcctggtcgcctccgtcctgctgcaggccgtcctttatccccggtttatgggcaccatatcagatgtaaagaccaatgtccagttgctgaaaggtcgtgtggacaacatcagcaccctggattctgaaattaaaaagaatagtgacggcatggaggcagctggcgttcagatccagatggtgaatgagagcctgggttatgtgcgttctcagttcctgaagttaaaaaccagtgtggagaaggccaacgcacagatccagatcttaacaagaagttgggaagaagtcagtaccttaaatgcccaaatcccagagttaaaaagtgatttggagaaagccagtgctttaaatacaaagatccgggcactccagggcagcttggagaatatgagcaagttgctcaaacgacaaaatgatattctacaggtggtttctcaaggctggaagtacttcaaggggaacttctattacttttctctcattccaaagacctggtatagtgccgagcagttctgtgtgtccaggaattcacacctgacctcggtgacctcagagagtgagcaggagtttctgtataaaacagcggggggactcatctactggattggcctgactaaagcagggatggaaggggactggtcctgggtggatgacacgccattcaacaaggtccaaagtgcgaggttctggattccaggtgagcccaacaatgctgggaacaatgaacactgtggcaatataaaggctccctcacttcaggcctggaatgatgccccatgtgacaaaacgtttcttttcatttgtaagcgaccctatgtcccatcagaaccgtga

SEQ ID NO: 5 - protein, 328 aa, Homo sapiens
MTVEKEAPDAHFTVDKQNISLWPREPPPKSGPSLVPGKTPTVRAALICLTLVLVASVLLQAVLYPRFMGTISDVKTNVQLLKGRVDNISTLDSEIKKNSDGMEAAGVQIQMVNESLGYVRSQFLKLKTSVEKANAQIQILTRSWEEVSTLNAQIPELKSDLEKASALNTKIRALQGSLENMSKLLKRQNDILQVVSQGWKYFKGNFYYFSLIPKTWYSAEQFCVSRNSHLTSVTSESEQEFLYKTAGGLIYWIGLTKAGMEGDWSWVDDTPFNKVQSARFWIPGEPNDAGNNEHCGNIKAPSLQAWNDAPCDKTFLFICKRPYVPSEP

SEQ ID NO: 6 - DNA, 987 bp, Homo sapiens
  source 1..987: mol_type = other DNA
atgactgtggagaaggaggcccctgatgcgcacttcactgtggacaaacagaacatctccctctggccccgagagcctcctcccaagtccggtccatctctggtcccggggaaaacacccacagtccgtgctgcattaatctgcctgacgctggtcctggtcgcctccgtcctgctgcaggccgtcctttatccccggtttatgggcaccatatcagatgtaaagaccaatgtccagttgctgaaaggtcgtgtggacaacatcagcaccctggattctgaaattaaaaagaatagtgacggcatggaggcagctggcgttcagatccagatggtgaatgagagcctgggttatgtgcgttctcagttcctgaagttaaaaaccagtgtggagaaggccaacgcacagatccagatcttaacaagaagttgggaagaagtcagtaccttaaatgcccaaatcccagagttaaaaagtgatttggagaaagccagtgctttaaatacaaagatccgggcactccagggcagcttggagaatatgagcaagttgctcaaacgacaaaatgatattctacaggtggtttctcaaggctggaagtacttcaaggggaacttctattacttttctctcattccaaagacctggtatagtgccgagcagttctgtgtgtccaggaattcacacctgacctcggtgacctcagagagtgagcaggagtttctgtataaaacagcggggggactcatctactggattggcctgactaaagcagggatggaaggggactggtcctgggtggatgacacgccattcaacaaggtccaaagtgcgaggttctggattccaggtgagcccaacgatgctgggaacaatgaacactgtggcaatataaaggctccctcacttcaggcctggaatgatgccccatgtgacaaaacgtttcttttcatttgtaagcgaccctatgtcccatcagaaccgtga

SEQ ID NO: 7 - protein, 328 aa, Homo sapiens
MTVEKEAPDAHFTVDKQNISLWPREPPPKSGPSLVPGKTPTVRAALICLTLVLVASVLLQAVLYPRFMGTISDVKTNVQLLKGRVDNISTLDSEIKKNSDGMEAAGVQIQMVNESLGYVRSQFLKLKTSVEKANAQIQILTRSWEEVSTLNAQIPELKSDLEKASALNTKIRALQGSLENMSKLLKRQNDILQVVSQGWKYFKGNFYYFSLIPKTWYSAEQFCVSRNSHLTSVTSESEQEFLYKTAGGLIYWIGLTKAGMEGDWSWVDDTPFNKVQSARFWIPGEPNNAGNNEHCGNIKAPSLQAWNDAPCDITFLFICKRPYVPSEP

SEQ ID NO: 8 - DNA, 987 bp, Homo sapiens
  source 1..987: mol_type = other DNA
atgactgtggagaaggaggcccctgatgcgcacttcactgtggacaaacagaacatctccctctggccccgagagcctcctcccaagtccggtccatctctggtcccggggaaaacacccacagtccgtgctgcattaatctgcctgacgctggtcctggtcgcctccgtcctgctgcaggccgtcctttatccccggtttatgggcaccatatcagatgtaaagaccaatgtccagttgctgaaaggtcgtgtggacaacatcagcaccctggattctgaaattaaaaagaatagtgacggcatggaggcagctggcgttcagatccagatggtgaatgagagcctgggttatgtgcgttctcagttcctgaagttaaaaaccagtgtggagaaggccaacgcacagatccagatcttaacaagaagttgggaagaagtcagtaccttaaatgcccaaatcccagagttaaaaagtgatttggagaaagccagtgctttaaatacaaagatccgggcactccagggcagcttggagaatatgagcaagttgctcaaacgacaaaatgatattctacaggtggtttctcaaggctggaagtacttcaaggggaacttctattacttttctctcattccaaagacctggtatagtgccgagcagttctgtgtgtccaggaattcacacctgacctcggtgacctcagagagtgagcaggagtttctgtataaaacagcggggggactcatctactggattggcctgactaaagcagggatggaaggggactggtcctgggtggatgacacgccattcaacaaggtccaaagtgcgaggttctggattccaggtgagcccaacaatgctgggaacaatgaacactgtggcaatataaaggctccctcacttcaggcctggaatgatgccccatgtgacataacgtttcttttcatttgtaagcgaccctatgtcccatcagaaccgtga

SEQ ID NO: 9 - protein, 328 aa, Homo sapiens [sequence omitted]

SEQ ID NO: 10 - DNA, 987 bp, Homo sapiens
  source 1..987: mol_type = other DNA
atgactgtggagaaggaggcccctgatgcgcacttcactgtggacaaacagaacatctccctctggccccgagagcctcctcccaagtccggtccatctctggtcccggggaaaacacccacagtccgtgctgcattaatctgcctgacgctggtcctggtcgcctccgtcctgctgcaggccgtcctttatccccggtttatgggcaccatatcagatgtaaagaccaatgtccagttgctgaaaggtcgtgtggacaacatcagcaccctggattctgaaattaaaaagaatagtgacggcatggaggcagctggcgttcagatccagatggtgaatgagagcctgggttatgtgcgttctcagttcctgaagttaaaaaccagtgtggagaaggccaacgcacagatccagatcttaacaagaagttgggaagaagtcagtaccttaaatgcccaaatcccagagttaaaaagtgatttggagaaagccagtgctttaaatacaaagatccgggcactccagggcagcttggagaatatgagcaagttgctcaaacgacaaaatgatattctacaggtggtttctcaaggctggaagtacttcaaggggaacttctattacttttctctcattccaaagacctggtatagtgccgagcagttctgtgtgtccaggaattcacacctgacctcggtgacctcagagagtgagcaggagtttctgtataaaacagcggggggactcatctactggattggcctgactaaagcagggatggaaggggactggtcctgggtggatgacacgccattcaacaaggtccaaagtgcgaggttctggattccaggtgagcccaacgatgctgggaacaatgaacactgtggcaatataaaggctccctcacttcaggcctggaatgatgccccatgtgacataacgtttcttttcatttgtaagcgaccctatgtcccatcagaaccgtga

SEQ ID NO: 11 - DNA, 855 bp, synthetic construct
  source 1..855: mol_type = other DNA
atggcacgcttcatgggcacgattagcgatgttaaaaccaatgttcaactgctgaaaggccgtgtggacaatatctcgaccctggatagtgaaattaagaaaaacagcgatggcatggaagcggccggtgtccagatccaaatggtgaatgaatcactgggttatgtccgttcgcagtttctgaaactgaaaaccagtgttgaaaaagcaaacgctcagattcaaatcctgacccgctcatgggaagaagtgtcgacgctgaatgcccagattccggaactgaaaagcgatctggaaaaagcgtctgccctgaacacgaaaatccgtgcactgcagggcagcctggaaaacatgtctaaactgctgaaacgccaaaatgacattctgcaggtggttagccaaggctggaaatacttcaagggtaacttctactacttcagtctgatcccgaaaacctggtactccgccgaacagttttgcgtgagtcgtaactcccatctgaccagcgttacgagcgaatctgaacaagaatttctgtataaaaccgctggcggtctgatttactggatcggtctgacgaaagcgggcatggagggtgattggtcatgggttgatgacaccccgtttaataaagtccagtcggcgcgtttctggattccgggcgaaccgaacaatgccggtaacaatgaacactgtggcaacatcaaagcaccgagcctgcaggcatggaatgatgctccgtgcgacaaaacgtttctgttcatttgtaaacgcccgtacgttccgtctgaaccgggatccgaaaatctgtacttccaaggttcggcgtggtcccatccgcagtttgaaaaataa

SEQ ID NO: 12 - protein, 331 aa, Mus musculus
MPEAEMKEEAPEAHFTVDKQNISLWPREPPPKQDLSPVLRKPLCICVAFTCLALVLVTSIVLQAVFYPRLMGKILDVKSDAQMLKGRVDNISTLGSDLKTERGRVDDAEVQMQIVNTTLKRVRSQILSLETSMKIANDQLQILTMSWGEVDSLSAKIPELKRDLDKASALNTKVQGLQNSLENVNKLLKQQSDILEMVARGWKYFSGNFYYFSRTPKTWYSAEQFCISRKAHLTSVSSESEQKFLYKAADGIPHWIGLTKAGSEGDWYWVDQTSFNKEQSRRFWIPGEPNNAGNNEHCANIRVSALKCWNDGPCDNTFLFICKRPYVQTTE

SEQ ID NO: 13 - DNA, 996 bp, Mus musculus
  source 1..996: mol_type = other DNA
atgccagaggcagagatgaaggaggaggctcccgaagcgcacttcacagtggacaaacagaacatctctctctggcctcgagagcctcctcccaagcaagatctgtctccagttctgaggaaacctctctgtatctgcgtggccttcacctgcctggcattggtgctggtcacctccattgtgcttcaggctgttttctatcctaggttgatgggcaaaatattggatgtgaagagtgatgcccagatgttgaaaggtcgtgtggacaacatcagcaccctgggttctgatcttaagactgaaagaggtcgtgtggacgatgctgaggttcagatgcagatagtgaacaccaccctcaagagggtgcgttctcagatcctgtctttggaaaccagcatgaagatagccaatgatcagctccagatattaacaatgagctggggagaggttgacagtctcagtgccaaaatcccagaactgaaaagagatctggataaagccagcgccttgaacacaaaggtccaaggactacagaacagcttggagaatgtcaacaagctgctcaaacaacagagtgacattctggagatggtggctcgaggctggaagtatttctcggggaacttctattacttttcacgcaccccaaagacctggtacagcgcagagcagttctgtatttctagaaaagctcacctgacctcagtgtcctcagaatcggaacaaaagtttctctacaaggcagcagatggaattccacactggattggacttaccaaagcagggagcgaaggggactggtactgggtggaccagacatcattcaacaaggagcaaagtaggaggttctggattccaggtgaacccaacaacgcagggaacaacgagcactgtgccaatatcagggtgtctgccctgaagtgctggaacgatggtccctgtgacaatacatttcttttcatctgcaagaggccctacgtccaaacaactgaatga